(19) United States
(12) Patent Application Publication
(10) Pub. No.: US 2026/0215077 A1
(43) Pub. Date: Jul. 23, 2026

(54) HETEROCYCLIC COMPOUND, LIGHT-EMITTING DEVICE AND ELECTRONIC DEVICE

(71) Applicant: Samsung Display Co., LTD., Yongin-si (KR)

(72) Inventors: Jung Hoon HAN, Yongin-si (KR); Do Jin KIM, Yongin-si (KR); Hyeong Min KIM, Yongin-si (KR); Hee Choon AHN, Yongin-si (KR); Hyun Ah UM, Yongin-si (KR)

(21) Appl. No.: 19/435,084

(22) Filed: Dec. 29, 2025

(30) Foreign Application Priority Data
Jan. 17, 2025 (KR) 10-2025-0007571

(51) Int. Cl. H10K 50/12; C07F 7/08; H10K 85/30; H10K 85/40; H10K 85/60; H10K 101/00; H10K 101/20

(52) U.S. Cl. CPC H10K 50/12; C07F 7/0812; H10K 85/346; H10K 85/40; H10K 85/654; H10K 85/656; H10K 85/6572; H10K 85/658; H10K 2101/20; H10K 2101/27

(57) ABSTRACT

A heterocyclic compound represented by Chemical Formula 1, a light-emitting device that includes the heterocyclic compound, and an electronic device that includes the light-emitting device are provided. The light-emitting device includes a first electrode, a second electrode, and an intermediate layer disposed between the first electrode and the second electrode. The intermediate layer includes the heterocyclic compound.

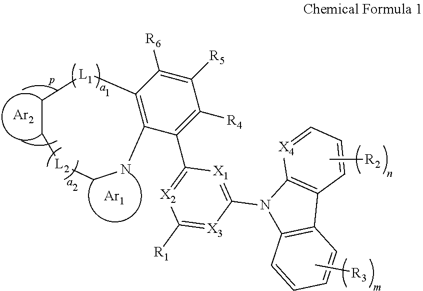

The variables in Chemical Formula 1 are as defined herein.

### CROSS-REFERENCE TO RELATED APPLICATION

This application claims priority to and the benefit of Korean Patent Application No. 10-2025-0007571 filed on Jan. 17, 2025 in the Korean Intellectual Property Office (KIPO), and all the benefits accruing therefrom under 35 U.S.C. § 119, the content of which is incorporated herein in its entirety by reference.

### TECHNICAL FIELD

Embodiments of the present application relate to a heterocyclic compound, a light-emitting device including the heterocyclic compound and an electronic device including the light-emitting device.

### BACKGROUND

An organic light-emitting diode (OLED) display has a self-luminous property and may provide improved viewing angle and contrast properties. Additionally, an OLED display may provide a high response speed and a high luminance.

An light-emitting device may include an emission layer disposed between a first electrode and a second electrode. A hole transferred from the first electrode and an electron transferred from the second electrode may be recombined in the emission layer to generate an exciton. Light may be emitted as the exciton is shifted from an excited state to a ground state.

The emission layer may include a host material and a dopant material for implementing the above-described light-emitting mechanism.

### SUMMARY

An aspect of the present disclosure includes a heterocyclic compound having improved spectrometry and life-emitting properties.

An aspect of the present disclosure includes a light-emitting device having improved light-emitting properties and reliability.

An aspect of the present disclosure includes an electronic device including the light-emitting device.

An aspect of the present disclosure includes a heterocyclic compound represented by Chemical Formula 1.

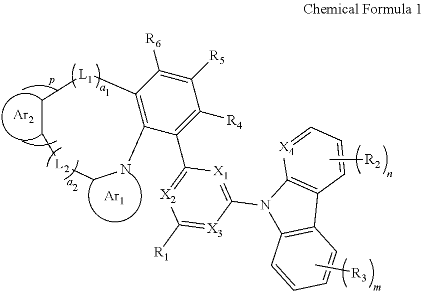

In Chemical Formula 1, Ar₁ may be a substituted or unsubstituted C₂-C₆₀ nitrogen-containing heteroaryl ring. Ar₂ may be a substituted or unsubstituted C₆-C₆₀ aryl ring or a substituted or unsubstituted C₂-C₆₀ heteroaryl ring. X₁ to X₄ may each independently be N or CR₇, provided that at least two of X¹ to X₃ are N.

R₁ to R₇ may each independently be hydrogen, deuterium, halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₁-C₆₀ heterocycloalkenyl group, a substituted or unsubstituted C₆-C₆₀ aryl group, a substituted or unsubstituted C₆-C₆₀ alkyl aryl group, a substituted or unsubstituted C₆-C₆₀ aryl alkyl group, a substituted or unsubstituted C₂-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₆-C₆₀ aryloxy group, a substituted or unsubstituted C₆-C₆₀ arylthio group, a substituted or unsubstituted C₈-C₆₀ condensed polycyclic group, —SiRR′R″, —P(═O) RR′, —NRR′, —BRR′, —C(═O) R or —S(═O)₂R.

Two or more adjacent groups among R₁ to R₇ may optionally be combined with each other to form a substituted or unsubstituted C₃-C₆₀ cycloalkyl ring, a substituted or unsubstituted C₅-C₆₀ cycloalkenyl ring, a substituted or unsubstituted C₃-C₆₀ heterocycloalkyl ring, a substituted or unsubstituted C₁-C₆₀ heterocycloalkenyl ring, a substituted or unsubstituted C₆-C₆₀ aryl ring or a substituted or unsubstituted C₂-C₆₀ heteroaryl ring

R, R′ and R″ may each independently be hydrogen, deuterium, halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₁-C₆₀ heterocycloalkenyl group, a substituted or unsubstituted C₆-C₆₀ aryl group, a substituted or unsubstituted C₆-C₆₀ alkyl aryl group, a substituted or unsubstituted C₆-C₆₀ aryl alkyl group, a substituted or unsubstituted C₂-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₆-C₆₀ aryloxy group, a substituted or unsubstituted C₆-C₆₀ arylthio group, or a substituted or unsubstituted C₈-C₆₀ condensed polycyclic group.

L₁ and L₂ may each independently be a direct bond, O, S, NR₈. R₈ may be a substituted or unsubstituted C₆-C₆₀ aryl group, a substituted or unsubstituted C₆-C₆₀ alkyl aryl group, a substituted or unsubstituted C₆-C₆₀ aryl alkyl group, a substituted or unsubstituted C₂-C₆₀ heteroaryl group, a substituted or unsubstituted C₂-C₆₀ alkyl heteroaryl group, a substituted or unsubstituted C₂-C₆₀ heteroaryl alkyl group, or may be is combined with Ar₁, Ar₂ or R₆ to form a substituted or unsubstituted C₂-C₆₀ nitrogen-containing heteroaryl ring.

The variables m, a₁ and a₂ may each independently be an integer of 0 to 4, and the variable n may be an integer of 0 to 3. The variable p is an integer of 1 to 3. When n is 2 or more, each R₂ is different or the same, when m is 2 or more, each R₃ is different or the same, when p is 2 or more, each Ar₂ is different or the same, when a₁ is 2 or more, each L₁ are different or the same, or when a₂ is 2 or more, each L₂ is different or the same.

A light-emitting device according to the present disclosure may include a first electrode, a second electrode, and an intermediate layer disposed between the first electrode and the second electrode. The intermediate layer may include the above-described heterocyclic compound of Chemical Formula 1.

An electronic device according to the present disclosure includes the above-described light-emitting device.

A heterocyclic compound according to the present inventive concepts may have a core structure including a heptagonal (seven) or more non-planar rings, and an intramolecular interaction may be suppressed. Accordingly, color purity and life-span properties of a light-emitting device including the heterocyclic compound may be improved.

The heterocyclic compound according to the present inventive concepts may include a twisted structure in which an electron donor and an electron acceptor are spaced apart from each other, and structures implementing HOMO and LUMO may be positionally separated. Accordingly, the heterocyclic compound may have TADF properties, and luminous efficiency of the light-emitting device may be improved.

The heterocyclic compound according to the present inventive concepts may have a robust molecular structure and improved chemical stability. Accordingly, the compound structure may be maintained for a long period in the light-emitting device, and life-span properties of the light-emitting device may be further improved.

### BRIEF DESCRIPTION OF THE DRAWINGS

FIGS. 1 to 6 are schematic cross-sectional views illustrating light-emitting devices in accordance with example embodiments.

FIG. 7 is a schematic cross-sectional view illustrating a display device in accordance with example embodiments.

FIG. 8 is a schematic cross-sectional view illustrating a display device in accordance with example embodiments.

FIG. 9 is a schematic cross-sectional view illustrating a stack construction of light-emitting structure in a display device in accordance with example embodiments.

FIG. 10 is a schematic cross-sectional view illustrating a display device in accordance with example embodiments.

FIG. 11 is a schematic cross-sectional view illustrating a display device in accordance with example embodiments.

FIG. 12 is a schematic exploded perspective view illustrating an electronic device in accordance with example embodiments.

FIG. 13 is a schematic view illustrating an electronic device in accordance with example embodiments.

FIG. 14 is a block diagram of an electronic device in accordance with an embodiment.

FIG. 15 are schematic diagrams of electronic devices in accordance with various embodiments.

### DETAILED DESCRIPTION OF THE EMBODIMENTS

According to embodiments of the present disclosure, a polycyclic heterocyclic compound having a nitrogen atom bonded to an ortho position with respect to an azine-based ring is provided. Further, a light-emitting device and an electronic device including the heterocyclic compound are provided.

The terminology used herein is for the purpose of describing one or more exemplary embodiments only and is not intended to be limiting. As used herein, the singular forms “a,” “an,” and “the” are intended to include the plural forms as well, unless the context clearly indicates otherwise. The term “or” means “and/or.” It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

It will be understood that, although the terms first, second, third, or the like may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of the present embodiments.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

It will be understood that when an element is referred to as being “on” another element, it can be directly in contact with the other element or intervening elements may be present therebetween. In contrast, when an element is referred to as being “directly on” another element, there are no intervening elements present.

Unless otherwise defined, all terms (including technical and scientific terms) used herein have the same meaning as commonly understood by one of ordinary skill in the art to which this general inventive concept belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±20%, ±10%, or ±5% of the stated value.

### Definition of Terminology

In the present specification, the term “substituted or unsubstituted” may refer to being substituted or unsubstituted by one or more substituent selected from the group consisting of, e.g., a deuterium atom, a halogen atom, a cyano group, a nitro group, an amino (amine) group, a silyl group, an oxy group, a thio group, a sulfinyl group, a sulfonyl group, a carbonyl group, an ester group, boron, a phosphine oxide group, a phosphine sulfide group, an alkyl group (e.g., a C₁-C₆₀, C₁-C₁₀ alkyl group), an alkenyl group (e.g., a C₂-C₆₀, C₂-C₁₀ alkenyl group), an alkynyl group (e.g., a C₂-C₆₀, C₂-C₁₀ alkynyl group), an alkoxy group (e.g., a C₁-C₆₀, C₁-C₁₀ alkoxy group), an alkylthio group (e.g. a C₁-C₆₀, C₁-C₁₀ alkylthio group), a hydrocarbon ring group, an aryl group (e.g., a C₆-C₆₀ aryl group), an alkyl aryl group (e.g. C₆-C₆₀ alkyl aryl group), an aryl alkyl group (e.g. C₆-C₆₀ aryl alkyl group), and a heterocyclic group (e.g., a C₁-C₆₀ heterocyclic group). For example, the term “substituted alkyl group” may refer to a group in which at least one of hydrogen atoms of the alkyl group is substituted with the above-described substituent, and thus the substituent is further bonded to a carbon atom of the alkyl group.

The substituent may include a combination of substituents selected from the groups described above. For example, at least one hydrogen atom in the alkyl group, the aryl group, etc., included as a substituent may be substituted with a deuterium atom, a halogen atom, a cyano group, a nitro group, an amino group, a silyl group, an oxy group, a thio group, a sulfinyl group, a sulfonyl group, a carbonyl group, an ester group, boron, a phosphine oxide group, a phosphine sulfide group, an alkyl group, an alkenyl group, an alkynyl group, a hydrocarbon ring group, an aryl group, a heterocyclic group, or a combination thereof.

In the substituents described above, a multivalent substituent such as an amino group, a phosphine sulfide group, a phosphine oxide group, a sulfinyl group, a sulfonyl group, an oxy group, a carbonyl group, an ester group, etc., may each independently be substituted with a C₁-C₁₀ alkyl group, a C₂-C₁₀ alkenyl group, a C₂-C₁₀ alkynyl group, a C₆-C₁₀ aryl group a C₆-C₁₀ alkyl aryl group, or a C₆-C₁₀ aryl alkyl group.

In the specification, in the term “substituted or unsubstituted Cₐ-C_{b} Y group” the range of a to b refers to the number of carbon atoms in an unsubstituted Y group, and may not include the number of carbon atoms of a substituent.

In the specification, an alkyl group may be a monovalent hydrocarbon group in which one hydrogen atom is removed from a linear or branched hydrocarbon group. Examples of an alkyl group may include a methyl group, an ethyl group, a propyl group, a sec-butyl group, a tert-butyl group, an iso-butyl group, a pentyl group, a neopentyl group, a 2-ethyl butyl group, a 3,3-dimethyl butyl group, a hexyl group, a heptyl group, an octyl group, etc.

In the specification, an alkylene group may be a divalent hydrocarbon group in which two hydrogen atoms are removed from a linear or branched hydrocarbon group.

In the specification, an alkenyl group may have the same skeleton as that of an alkyl group, and may be a monovalent hydrocarbon group that includes at least one carbon-carbon double bond. In the specification, an alkenylene group may be a divalent hydrocarbon group in which one hydrogen atom is further removed from an alkenyl group.

In the specification, an alkynyl group may have the same skeleton as that of an alkyl group, and may be a monovalent hydrocarbon group that includes at least one carbon-carbon triple bond. In the specification, an alkynylene group may be a divalent hydrocarbon group in which one hydrogen atom is further removed from an alkynyl group.

In the specification, an aryl group may be a monovalent hydrocarbon group in which one hydrogen atom is removed from a hydrocarbon group having an aromatic structure. The definition of an aryl group may also encompass a group in which multiple aromatic rings are directly connected, such as a biphenyl group. Examples of an aryl group may include, e.g., a phenyl group, a naphthyl group, an anthracenyl group, a phenanthrenyl group, a pyrenyl group, a fluorenyl group, a tetracenyl group, a biphenyl group, a terphenyl group, a quaterphenyl group, a chrysenyl group, etc.

In the specification, a group in which two or more aryl rings are condensed to each other or linked to each other by an alicyclic hydrocarbon ring, such as a fluorenyl group, can be encompassed in the definition of an aryl group.

For example, a biphenyl group may be interpreted as an aryl group, or may be interpreted as a phenyl group that is substituted with a phenyl group.

In the specification, an arylene group may be a divalent hydrocarbon group in which two hydrogen atoms are removed from an aryl group.

In the specification, a heteroaryl group may be a monovalent group having an aromatic structure that includes at least one heteroatom such as B, O, P, S, Si, Se, and Ge as a ring-forming atom. In the specification, a heteroarylene group may be a divalent group having an aromatic structure that includes at least one heteroatom such as B, O, P, S, Si, Se, and Ge as a ring-forming atom. When a heteroaryl group or a heteroarylene group includes two or more heteroatoms, the two or more heteroatoms may be the same as or different from each other. In the specification, a heterocycloalkenyl group may be a divalent group that includes at least one heteroatom such as B, O, P, S, Si, Se, and Ge, and has double bond. The double bond may be a carbon-carbon double bond or a carbon-heteroatom double bond.

In the specification, an aryloxy group may be -OA₁₀₂ (wherein A₁₀₂ is the C₆-C₆₀ aryl group), and an alkylthio group may be a monovalent group represented by -SA₁₀₁ (wherein A₁₀₁ is the C₁-C₆₀ alkyl group).

In the specification, an alkyl aryl group is an aryl group connected to a moiety by an alkyl group, e.g., a benzyl group is an alkyl aryl group. Similarly, an alkyl heteroaryl group is a heteroaryl group connected to a moiety via an alkyl group.

In the specification, a group in which two or more aryl rings are condensed or linked to a non-aromatic heterocyclic ring, such as a carbazole group, can also be encompassed in the definition of a heteroaryl group.

In the specification, the term “cyclic group” may encompass a monocyclic group or a polycyclic group, and may also encompass an alicyclic ring or an aromatic ring.

In the specification, the term “polycyclic group” may be a group in which two or more rings are connected to each other or condensed to each other through one or more atoms. For example, a polycyclic structure may include a bicyclic structure through a bridge carbon, a spiro structure, a fused structure, etc.

In the specification, the term “condensed group” or “condensed ring structure” may each be a group in which two or more adjacent rings share two or more atoms among the above-described polycyclic structures. Examples of a condensed ring structure may include naphthalene, anthracene, phenanthrene, fluorene, pyrene, benzopyrene, pentacene, polyacene, helicene, etc.

In the specification, the term “carbocyclic group (e.g., C₃-C₆₀ carbocyclic group)” may be a cyclic group in which carbon atoms are the only ring-forming atoms. In the specification, a heterocyclic group (e.g., a C₁-C₆₀ heterocyclic group) may be a cyclic group that includes at least one heteroatom as a ring-forming atom, in addition to carbon atoms.

In the specification, a carbocyclic group and a heterocyclic group may each independently be a monocyclic group that consists of one ring or a polycyclic group in which two or more rings are condensed with each other.

### Heterocyclic Compound

The heterocyclic compound according to embodiments of the present disclosure may be represented by Chemical Formula 1.

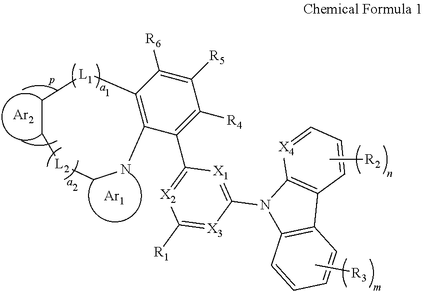

In Chemical Formula 1, Ar₁ may be a substituted or unsubstituted C₂-C₆₀ nitrogen-containing heteroaryl ring. In example embodiments, Ar₁ may be a substituted or unsubstituted C₂-C₄₀ nitrogen-containing heteroaryl ring. In some embodiments, Ar₁ may be a substituted or unsubstituted C₂-C₂₀ nitrogen-containing heteroaryl ring.

In example embodiments, Ar₁ may be a deuterium-substituted or unsubstituted C₂-C₆₀ nitrogen-containing heteroaryl ring. In some embodiments, Ar₁ may be a deuterium-substituted or unsubstituted C₂-C₄₀ nitrogen-containing heteroaryl ring. For example, Ar₁ may be a deuterium-substituted or unsubstituted C₂-C₂₀ nitrogen-containing heteroaryl ring.

For example, Ar₁ may be selected from the group consisting of a substituted or unsubstituted indoline ring, a substituted or unsubstituted indole ring, a substituted or unsubstituted isoindole ring, a substituted or unsubstituted indazole ring, a substituted or unsubstituted benzimidazole ring, a substituted or unsubstituted purine ring, a substituted or unsubstituted tetrahydroquinoline ring, a substituted or unsubstituted dihydroquinoline ring, and a substituted or unsubstituted carbazole ring.

For example, Ar₁ may be selected from the group consisting of a deuterium-substituted or unsubstituted indoline ring, a deuterium-substituted or unsubstituted indole ring, a deuterium-substituted or unsubstituted isoindole ring, a deuterium-substituted or unsubstituted indazole ring, a deuterium-substituted or unsubstituted benzimidazole ring, a deuterium-substituted or unsubstituted purine ring, a deuterium-substituted or unsubstituted tetrahydroquinoline ring, a deuterium-substituted or unsubstituted dihydroquinoline ring, and a deuterium-substituted or unsubstituted carbazole ring.

In Chemical Formula 1, Ar₂ may be a substituted or unsubstituted C₆-C₆₀ aryl ring or a substituted or unsubstituted C₂-C₆₀ heteroaryl ring. In example embodiments, Ar₂ may be a substituted or unsubstituted C₆-C₄₀ aryl ring or a substituted or unsubstituted C₂-C₄₀ heteroaryl ring. In some embodiments, Ar₂ may be a substituted or unsubstituted C₆-C₂₀ aryl ring or a substituted or unsubstituted C₂-C₂₀ heteroaryl ring.

In example embodiments, Ar₂ may be a deuterium-substituted or unsubstituted C₆-C₆₀ aryl ring or a deuterium-substituted or unsubstituted C₂-C₆₀ heteroaryl ring. In some embodiments, Ar₂ may be a deuterium-substituted or unsubstituted C₆-C₄₀ aryl ring or a deuterium-substituted or unsubstituted C₂-C₄₀ heteroaryl ring. For example, Ar₂ may be a deuterium-substituted or unsubstituted C₆-C₂₀ aryl ring or a deuterium-substituted or unsubstituted C₂-C₂₀ heteroaryl ring.

For example, Ar₂ may be selected from the group consisting of a substituted or unsubstituted phenyl ring, a substituted or unsubstituted naphthalene ring, a substituted or unsubstituted anthracene ring, a substituted or unsubstituted pyridine ring, a substituted or unsubstituted pyridazine ring, a substituted or unsubstituted pyrazine ring, a substituted or unsubstituted triazine ring, a substituted or unsubstituted indene ring, a substituted or unsubstituted dihydroindene ring, a substituted or unsubstituted fluorene ring, a substituted or unsubstituted spirobifluorene ring, a substituted or unsubstituted indoline ring, a substituted or unsubstituted indole ring, a substituted or unsubstituted isoindole ring, a substituted or unsubstituted indolizine ring, a substituted or unsubstituted indazole ring, a substituted or unsubstituted benzimidazole ring, a substituted or unsubstituted azaindole ring, a substituted or unsubstituted benzofuran ring, a substituted or unsubstituted isobenzofuran ring, a substituted or unsubstituted benzothiophene ring, a substituted or unsubstituted tetrahydroquinoline ring, a substituted or unsubstituted dihydroquinoline ring, a substituted or unsubstituted quinoline ring, a substituted or unsubstituted isoquinoline ring, and a substituted or unsubstituted quinoxaline ring.

For example, Ar₂ may be selected from the group consisting of a deuterium-substituted or unsubstituted a phenyl ring, a deuterium-substituted or unsubstituted naphthalene ring, a deuterium-substituted or unsubstituted anthracene ring, a deuterium-substituted or unsubstituted pyridine ring, a deuterium-substituted or unsubstituted pyridazine ring, a deuterium-substituted or unsubstituted pyrazine ring, a deuterium-substituted or unsubstituted triazine ring, a deuterium-substituted or unsubstituted indene ring, a deuterium-substituted or unsubstituted dihydroindene ring, a deuterium-substituted or unsubstituted fluorene ring, a deuterium-substituted or unsubstituted spirobifluorene ring, a deuterium-substituted or unsubstituted indoline ring, a deuterium-substituted or unsubstituted indole ring, a deuterium-substituted or unsubstituted isoindole ring, a deuterium-substituted or unsubstituted indolizine ring, a deuterium-substituted or unsubstituted indazole ring, a deuterium-substituted or unsubstituted benzimidazole ring, a deuterium-substituted or unsubstituted azaindole ring, a deuterium-substituted or unsubstituted benzofuran ring, a deuterium-substituted or unsubstituted isobenzofuran ring, a deuterium-substituted or unsubstituted benzothiophene ring, a deuterium-substituted or unsubstituted tetrahydroquinoline ring, a deuterium-substituted or unsubstituted dihydroquinoline ring, a deuterium-substituted or unsubstituted quinoline ring, a deuterium-substituted or unsubstituted isoquinoline ring, and a deuterium-substituted or unsubstituted quinoxaline ring.

In Chemical Formula 1, X₁ to X₄ may each independently be N or CR₇, provided that at least two of X₁ to X₃ are N. For example, X₁ to X₃ may be N. For example, X₁ and X₃ may be N, and X₂ may be CR₇. For example, X₁ and X₂ may be N, and X₃ may be CR₇. For example, X₂ and X₃ may be N, and X₁ may be CR₇.

In Chemical Formula 1, R₁ to R₇ may each independently be hydrogen, deuterium, halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₁-C₆₀ heterocycloalkenyl group, a substituted or unsubstituted C₆-C₆₀ aryl group, a substituted or unsubstituted C₆-C₆₀ alkyl aryl group, a substituted or unsubstituted C₆-C₆₀ aryl alkyl group, a substituted or unsubstituted C₂-C₆₀ heteroaryl group, a substituted or unsubstituted C₂-C₆₀ alkyl heteroaryl group, or a substituted or unsubstituted C₂-C₆₀ heteroaryl alkyl group, a substituted or unsubstituted C₆-C₆₀ aryloxy group, a substituted or unsubstituted C₆-C₆₀ arylthio group, a substituted or unsubstituted C₈-C₆₀ condensed polycyclic group, —SiRR′R″, —P(═O) RR′, —NRR′, —BRR′, —C(═O) R or —S(═O)₂R; or two or more adjacent groups among R₁ to R₇ may be combined with each other to form a substituted or unsubstituted C₃-C₆₀ cycloalkyl ring, a substituted or unsubstituted C₅-C₆₀ cycloalkenyl ring, a substituted or unsubstituted C₃-C₆₀ heterocycloalkyl ring, a substituted or unsubstituted C₁-C₆₀ heterocycloalkenyl ring, a substituted or unsubstituted C₆-C₆₀ aryl ring or a substituted or unsubstituted C₂-C₆₀ heteroaryl ring.

R, R′ and R″ may each be independently hydrogen, deuterium, halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₁-C₆₀ heterocycloalkenyl group, a substituted or unsubstituted C₆-C₆₀ aryl group, a substituted or unsubstituted C₆-C₆₀ alkyl aryl group, a substituted or unsubstituted C₆-C₆₀ aryl alkyl group, a substituted or unsubstituted C₂-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₆-C₆₀ aryloxy group, a substituted or unsubstituted C₆-C₆₀ arylthio group, or a substituted or unsubstituted C₈-C₆₀ condensed polycyclic group.

For example, R₁ may be —SiRR′R″, a substituted or unsubstituted C₆-C₆₀ aryl group, a substituted or unsubstituted C₆-C₆₀ alkyl aryl group, a substituted or unsubstituted C₆-C₆₀ aryl alkyl group, a substituted or unsubstituted C₂-C₆₀ heteroaryl group, a substituted or unsubstituted C₂-C₆₀ alkyl heteroaryl group, or a substituted or unsubstituted C₂-C₆₀ heteroaryl alkyl group.

For example, R₁ may be —SiRR′R″, a C₆-C₆₀ aryl group substituted with one or more-SiRR′R″, or a C₂-C₆₀ heteroaryl group substituted with one or more-SiRR′R″.

For example, R₂ and R₃ may each independently be hydrogen, deuterium, a substituted or unsubstituted C₁-C₁₀ alkyl group, a substituted or unsubstituted C₆-C₂₀ aryl group, a substituted or unsubstituted C₆-C₂₀ alkyl aryl group, or a substituted or unsubstituted C₆-C₂₀ aryl alkyl group. For example, R₄ to R₇ may each independently be hydrogen or deuterium.

For example, R₆ may be combined with L₁ to form a ring.

In Chemical Formula 1, L₁ and L₂ may each independently be a direct bond, O, S, NR₈. For example, L₁ and L₂ may each be a direct bond. For example, L₁ may be O, S or NR₈, and L₂ may be a direct bond. For example, L₂ may be O, S or NR₈, and L₂ may be a direct bond.

In Chemical Formula 1, R₈ may be a substituted or unsubstituted C₆-C₆₀ aryl group, a substituted or unsubstituted C₆-C₆₀ alkyl aryl group, a substituted or unsubstituted C₆-C₆₀ aryl alkyl group, a substituted or unsubstituted C₂-C₆₀ heteroaryl group, a substituted or unsubstituted C₂-C₆₀ alkyl heteroaryl group, or a substituted or unsubstituted C₂-C₆₀ heteroaryl alkyl group, or may be combined with Ar₁, Ar₂ or R₆ to form a substituted or unsubstituted C₂-C₆₀ nitrogen-containing heteroaryl ring.

In example embodiments, R₈ may be a substituted or unsubstituted C₆-C₄₀ aryl group, a substituted or unsubstituted C₆-C₄₀ alkyl aryl group, a substituted or unsubstituted C₆-C₄₀ aryl alkyl group, a substituted or unsubstituted C₂-C₄₀ heteroaryl group, a substituted or unsubstituted C₂-C₄₀ alkyl heteroaryl group, or a substituted or unsubstituted C₂-C₄₀ heteroaryl alkyl group. In some embodiments, R₈ may be a substituted or unsubstituted C₆-C₂₀ aryl group, a substituted or unsubstituted C₆-C₂₀ alkyl aryl group, a substituted or unsubstituted C₆-C₂₀ aryl alkyl group, a substituted or unsubstituted C₂-C₂₀ heteroaryl group, a substituted or unsubstituted C₂-C₂₀ alkyl heteroaryl group, or a substituted or unsubstituted C₂-C₂₀ heteroaryl alkyl group.

In example embodiments, R₈ may be a deuterium-substituted or unsubstituted C₆-C₆₀ aryl group, a deuterium-substituted or unsubstituted C₆-C₆₀ alkyl aryl group, a deuterium-substituted or unsubstituted C₆-C₆₀ aryl alkyl group, a deuterium-substituted or unsubstituted C₂-C₆₀ heteroaryl group, a deuterium-substituted or unsubstituted C₂-C₆₀ alkyl heteroaryl group, or a deuterium-substituted or unsubstituted C₂-C₆₀ heteroaryl alkyl group. In some embodiments, R₈ may be a deuterium-substituted or unsubstituted C₆-C₄₀ aryl group, a deuterium-substituted or unsubstituted C₆-C₄₀ alkyl aryl group, a deuterium-substituted or unsubstituted C₆-C₄₀ aryl alkyl group, a deuterium-substituted or unsubstituted C₂-C₄₀ heteroaryl group, a deuterium-substituted or unsubstituted C₂-C₄₀ alkyl heteroaryl group, or a deuterium-substituted or unsubstituted C₂-C₄₀ heteroaryl alkyl group. For example, R₈ may be a deuterium-substituted or unsubstituted C₆-C₂₀ aryl group, a deuterium-substituted or unsubstituted C₆-C₂₀ alkyl aryl group, a deuterium-substituted or unsubstituted C₆-C₂₀ aryl alkyl group, a deuterium-substituted or unsubstituted C₂-C₂₀ heteroaryl group, a deuterium-substituted or unsubstituted C₂-C₂₀ alkyl heteroaryl group, or a deuterium-substituted or unsubstituted C₂-C₂₀ heteroaryl alkyl group.

For example, R₈ may be selected from the group consisting of a substituted or unsubstituted phenyl group, a substituted or unsubstituted naphthalene group, a substituted or unsubstituted anthracene group, a substituted or unsubstituted pyridine group, a substituted or unsubstituted pyridazine group, a substituted or unsubstituted pyrazine group, a substituted or unsubstituted triazine group, a substituted or unsubstituted indene group, a substituted or unsubstituted dihydroindene ring, a substituted or unsubstituted fluorene group, a substituted or unsubstituted spirobifluorene group, a substituted or unsubstituted indoline group, a substituted or unsubstituted indole group, a substituted or unsubstituted isoindole group, a substituted or unsubstituted indolizine group, a substituted or unsubstituted indazole group, a substituted or unsubstituted benzimidazole group, a substituted or unsubstituted azaindole group, a substituted or unsubstituted benzofuran group, a substituted or unsubstituted isobenzofuran group, a substituted or unsubstituted benzothiophene group, a substituted or unsubstituted tetrahydroquinoline group, a substituted or unsubstituted dihydroquinoline group, a substituted or unsubstituted quinoline group, a substituted or unsubstituted isoquinoline group, and a substituted or unsubstituted quinoxaline group.

For example, R₈ may be selected from the group consisting of a deuterium-substituted or unsubstituted phenyl group, a deuterium-substituted or unsubstituted naphthalene group, a deuterium-substituted or unsubstituted anthracene group, a deuterium-substituted or unsubstituted pyridine group, a deuterium-substituted or unsubstituted pyridazine group, a deuterium-substituted or unsubstituted pyrazine group, a deuterium-substituted or unsubstituted triazine group, a deuterium-substituted or unsubstituted indene group, a deuterium-substituted or unsubstituted dihydroindene ring, a deuterium-substituted or unsubstituted fluorene group, a deuterium-substituted or unsubstituted spirobifluorene group, a deuterium-substituted or unsubstituted indoline group, a deuterium-substituted or unsubstituted indole group, a deuterium-substituted or unsubstituted isoindole group, a deuterium-substituted or unsubstituted indolizine group, a deuterium-substituted or unsubstituted indazole group, a deuterium-substituted or unsubstituted benzimidazole group, a deuterium-substituted or unsubstituted azaindole group, a deuterium-substituted or unsubstituted benzofuran group, a deuterium-substituted or unsubstituted isobenzofuran group, a deuterium-substituted or unsubstituted benzothiophene group, a deuterium-substituted or unsubstituted tetrahydroquinoline group, a deuterium-substituted or unsubstituted dihydroquinoline group, a deuterium-substituted or unsubstituted quinoline group, a deuterium-substituted or unsubstituted isoquinoline group, and a deuterium-substituted or unsubstituted quinoxaline group.

In example embodiments, R₈ may be combined with Ar₁, Ar₂ or R₆ of Chemical Formula 1 to form a substituted or unsubstituted C₂-C₄₀ nitrogen-containing heteroaryl ring. In some embodiments, R& may be combined with Ar₁, Ar₂ or R₆ of Chemical Formula 1 to form a substituted or unsubstituted C₂-C₂₀ nitrogen-containing heteroaryl ring.

In example embodiments, R₈ may be combined with Ar₁, Ar₂ or R₆ of Chemical Formula 1 to form a deuterium-substituted or unsubstituted C₂-C₆₀ nitrogen-containing heteroaryl ring. In some embodiments, R₈ may be combined with Ar₁, Ar₂ or R₆ of Chemical Formula 1 to form a deuterium-substituted or unsubstituted C₂-C₄₀ nitrogen-containing heteroaryl ring. For example, R₈ may be combined with Ar₁, Ar₂ or R₆ of Chemical Formula 1 to form a deuterium-substituted or unsubstituted C₂-C₂₀ nitrogen-containing heteroaryl ring.

For example, R₈ may be combined with Ar₁, Ar₂ or R₆ of Chemical Formula 1 to form a ring selected from the group consisting of a substituted or unsubstituted indoline ring, a substituted or unsubstituted indole ring, a substituted or unsubstituted isoindole ring, a substituted or unsubstituted indazole ring, a substituted or unsubstituted benzimidazole ring, a substituted or unsubstituted purine ring, a substituted or unsubstituted tetrahydroquinoline ring, a substituted or unsubstituted dihydroquinoline ring, and a substituted or unsubstituted carbazole ring.

For example, R₈ may be combined with Ar₁, Ar₂ or R₆ of Chemical Formula 1 to form a ring selected from the group consisting of a deuterium-substituted or unsubstituted indoline ring, a deuterium-substituted or unsubstituted indole ring, a deuterium-substituted or unsubstituted isoindole ring, a deuterium-substituted or unsubstituted indazole ring, a deuterium-substituted or unsubstituted benzimidazole ring, a deuterium-substituted or unsubstituted purine ring, a deuterium-substituted or unsubstituted tetrahydroquinoline ring, a deuterium-substituted or unsubstituted dihydroquinoline ring, and a deuterium-substituted or unsubstituted carbazole ring.

In Chemical Formula 1, m, a₁ and a₂ may each independently be an integer of 0 to 4 and n may be an integer of 0 to 3. For example, n, m, a₁ and a₂ may each independently be 0, 1 or 2. In Chemical Formula 1, p is an integer from 1 to 3. For example, p may be 1 or 2.

In Chemical Formula 1, when n, m, p, a₁ and a₂ are each independently 2 or more, a plurality of each of R₂, R₃, Ar₂, L₁ and L₂ may be different from or the same as each other R₂, R₃, Ar₂, L₁ and L₂, respectively. For example, when n is 2 or more, a plurality of R₂ may be different from or the same as each other. For example, when m is 2 or more, a plurality of R₃ may be different from or the same as each other. For example, when p is 2 or more, a plurality of Ar₂ may be different from or the same as each other. For example, when a₁ is 2 or more, a plurality of L₁ may be different from or the same as each other. For example, when a₂ is 2 or more, a plurality of L₂ may be different from or the same as each other.

In some embodiments, at least one of a₁ and a₂ may not be 0, i.e., at least one or both of a₁ and a₂ may be more than 1 or equal to 1.

In example embodiments, the heterocyclic compound may be represented by Chemical Formula 1-1 below.

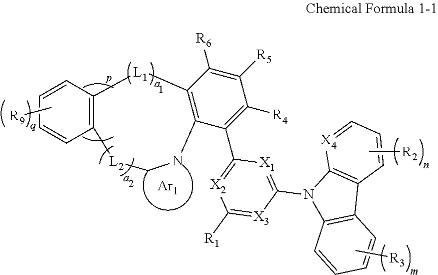

In Chemical Formula 1-1, the definitions of Ar₁, X₁ to X₄, R₁ to R₆, L₁, L₂, n, m, p, a₁ and a₂ may be the same as those described above in connection with Chemical Formula 1.

In Chemical Formula 1-1, R₉ may be hydrogen, deuterium, halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₁-C₆₀ heterocycloalkenyl group, a substituted or unsubstituted C₆-C₆₀ aryl group, a substituted or unsubstituted C₆-C₆₀ alkyl aryl group, a substituted or unsubstituted C₆-C₆₀ aryl alkyl group, a substituted or unsubstituted C₂-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₆-C₆₀ aryloxy group, a substituted or unsubstituted C₆-C₆₀ arylthio group, a substituted or unsubstituted C₈-C₆₀ condensed polycyclic group, —SiRR′R″, —P(═O)RR′, —NRR′, —BRR′, —C(═O) R or —S(═O)₂R.

When q is 2 or more, two or more adjacent groups R₉ may be combined with each other to form a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₁-C₆₀ heterocycloalkenyl group, a substituted or unsubstituted C₆-C₆₀ aryl group, a substituted or unsubstituted C₆-C₆₀ alkyl aryl group, a substituted or unsubstituted C₆-C₆₀ aryl alkyl group, or a substituted or unsubstituted C₂-C₆₀ heteroaryl group.

R, R′ and R″ may each independently be hydrogen, deuterium, halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₁-C₆₀ heterocycloalkenyl group, a substituted or unsubstituted C₆-C₆₀ aryl group, a substituted or unsubstituted C₆-C₆₀ alkyl aryl group, a substituted or unsubstituted C₆-C₆₀ aryl alkyl group, a substituted or unsubstituted C₂-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₆-C₆₀ aryloxy group, a substituted or unsubstituted C₆-C₆₀ arylthio group, or a substituted or unsubstituted C₈-C₆₀ condensed polycyclic group.

For example, R₉ may be hydrogen, deuterium, a substituted or unsubstituted C₆-C₆₀ aryl group, a substituted or unsubstituted C₆-C₆₀ alkyl aryl group, or a substituted or unsubstituted C₆-C₆₀ aryl alkyl group.

In Chemical Formula 1-1, q may be an integer of 0 to 4. For example, q may be 0, 1, or 2. When q is 2 or more, each R₉ may be the same or different.

In example embodiments, the heterocyclic compound may be represented by Chemical Formula 1-2 below.

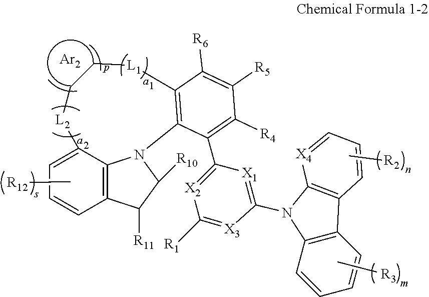

In Chemical Formula 1-2, the definitions of Ar₂, X₁ to X₄, R₁ to R₆, L₁, L₂, n, m, p, a₁ and a₂ may be the same as those described above in connection with Chemical Formula 1.

In Chemical Formula 1-2, R₁₀ to R₁₂ may each independently be hydrogen, deuterium, halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₁-C₆₀ heterocycloalkenyl group, a substituted or unsubstituted C₆-C₆₀ aryl group, a substituted or unsubstituted C₆-C₆₀ alkyl aryl group, a substituted or unsubstituted C₆-C₆₀ aryl alkyl group, a substituted or unsubstituted C₂-C₆₀ heteroaryl group, a substituted or unsubstituted C₂-C₆₀ alkyl heteroaryl group, or a substituted or unsubstituted C₂-C₆₀ heteroaryl alkyl group, a substituted or unsubstituted C₆-C₆₀ aryloxy group, a substituted or unsubstituted C₆-C₆₀ arylthio group, a substituted or unsubstituted C₈-C₆₀ condensed polycyclic group, —SiRR′R″, —P(═O) RR′, —NRR′, —BRR′, —C(═O) R or —S(═O)₂R.

Two or more adjacent ones of R₁₀ to R₁₂ may be combined with each other to form a substituted or unsubstituted C₃-C₆₀ cycloalkyl ring, a substituted or unsubstituted C₅-C₆₀ cycloalkenyl ring, a substituted or unsubstituted C₃-C₆₀ heterocycloalkyl ring, a substituted or unsubstituted C₁-C₆₀ heterocycloalkenyl ring, a substituted or unsubstituted C₆-C₆₀ aryl ring, or a substituted or unsubstituted C₂-C₆₀ heteroaryl ring.

R, R′ and R″ may each independently be hydrogen, deuterium, halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₁-C₆₀ heterocycloalkenyl group, a substituted or unsubstituted C₆-C₆₀ aryl group, a substituted or unsubstituted C₆-C₆₀ alkyl aryl group, a substituted or unsubstituted C₆-C₆₀ aryl alkyl group, a substituted or unsubstituted C₂-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₆-C₆₀ aryloxy group, a substituted or unsubstituted C₆-C₆₀ arylthio group, or a substituted or unsubstituted C₈-C₆₀ condensed polycyclic group.

For example, R₁₀ and R₁₁ may be combined with each other to form a substituted or unsubstituted C₃-C₄₀ cycloalkyl ring, a substituted or unsubstituted C₅-C₄₀ cycloalkenyl ring, a substituted or unsubstituted C₃-C₄₀ heterocycloalkyl ring, a substituted or unsubstituted C₁-C₄₀ heterocycloalkenyl ring, a substituted or unsubstituted C₆-C₄₀ aryl ring, or a substituted or unsubstituted C₂-C₄₀ heteroaryl ring.

For example, R₁₀ and R₁₁ may be combined with each other to form a substituted or unsubstituted C₆-C₂₀ aryl ring.

For example, R₁₂ may be hydrogen or deuterium.

In Chemical Formula 1-2, s may be an integer from 0 to 3. For example, s may be 0 or 1. When s is 2 or more, multiple R₁₂ may be the same as or different from each other.

In example embodiments, the heterocyclic compound may be represented by any one of Chemical Formulae 1-3 to 1-6.

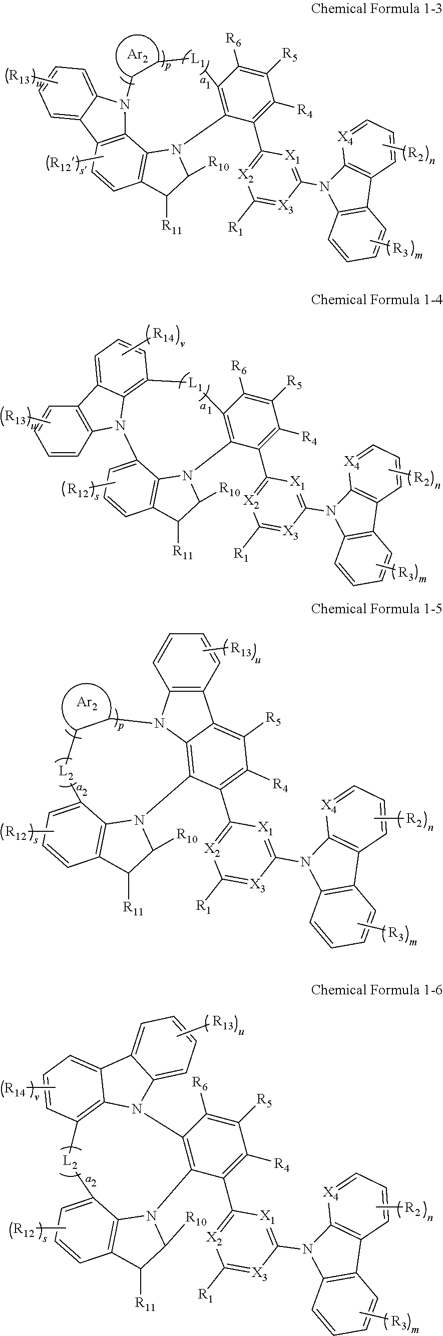

In Chemical Formulae 1-3 to 1-6, the definitions of Ar₂, X₁ to X₄, R₁ to R₆, L₁, n, m, p, a₁ and a₂ may be the same as those described above in connection with Chemical Formula 1.

The definitions of R₁₀ to R₁₂ may be the same as those described above in connection with Chemical Formula 1-2.

R₁₃, R₁₄ and R_{12′} may each independently be hydrogen, deuterium, halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₁-C₆₀ heterocycloalkenyl group, a substituted or unsubstituted C₆-C₆₀ aryl group, a substituted or unsubstituted C₆-C₆₀ alkyl aryl group, a substituted or unsubstituted C₆-C₆₀ aryl alkyl group, a substituted or unsubstituted C₂-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₆-C₆₀ aryloxy group, a substituted or unsubstituted C₆-C₆₀ arylthio group, a substituted or unsubstituted C₈-C₆₀ condensed polycyclic group, —SiRR′R″, —P(═O)RR′, —NRR′, —BRR′, —C(═O) R or —S(═O)₂R.

Two or more adjacent ones of R₁₃, R₁₄ and R₁₂′ may be combined with each other to form a substituted or unsubstituted C₃-C₆₀ cycloalkyl ring, a substituted or unsubstituted C₅-C₆₀ cycloalkenyl ring, a substituted or unsubstituted C₃-C₆₀ heterocycloalkyl ring, a substituted or unsubstituted C₁-C₆₀ heterocycloalkenyl ring, a substituted or unsubstituted C₆-C₆₀ aryl ring, or a substituted or unsubstituted C₂-C₆₀ heteroaryl ring.

R, R′ and R″ may each independently be hydrogen, deuterium, halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₁-C₆₀ heterocycloalkenyl group, a substituted or unsubstituted C₆-C₆₀ aryl group, a substituted or unsubstituted C₆-C₆₀ alkyl aryl group, a substituted or unsubstituted C₆-C₆₀ aryl alkyl group, a substituted or unsubstituted C₂-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₆-C₆₀ aryloxy group, a substituted or unsubstituted C₆-C₆₀ arylthio group, or a substituted or unsubstituted C₈-C₆₀ condensed polycyclic group.

In Chemical Formulae 1-3 to 1-6, s and v may each independently be an integer of 0 to 3, u may be an integer of 0 to 4, and s′ may be 0, 1, or 2. For example, s, v, u, and s′ may each independently be 0 or 1.

When s, v, and u are each independently 2 or more, a plurality of each of R₁₂ to R₁₄ may be the same as or different from each other. For example, when s is 2 or more, a plurality of R₁₂ may be the same as or different from each other. For example, when u is 2 or more, a plurality of R₁₃ may be the same as or different from each other. For example, when v is 2 or more, a plurality of R₁₄ may be the same as or different from each other.

When s′ is 2, a plurality of R₁₂′ may be the same as or different from each other.

In example embodiments, the heterocyclic compound may be represented by any one of Chemical Formulae 1-7 to 1-9.

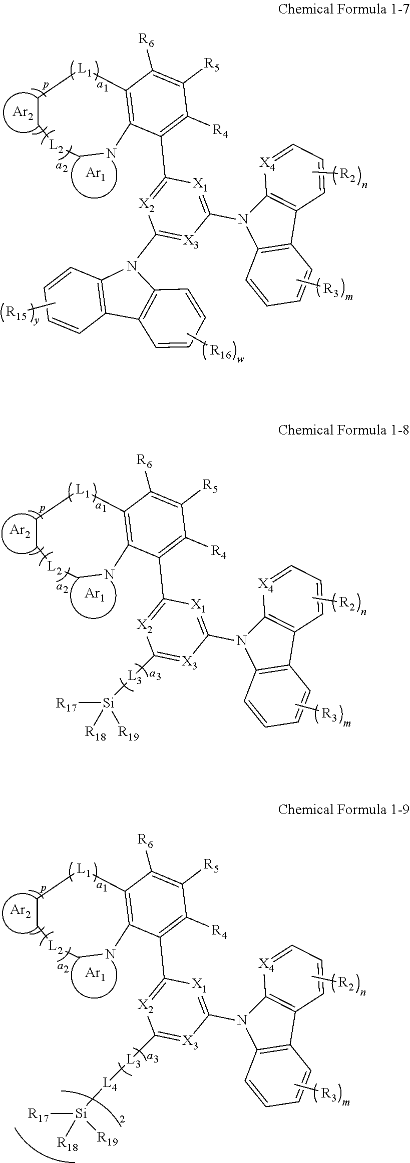

In Chemical Formulae 1-7 to 1-9, the definitions of Ar₁, Ar₂, X₁ to X₄, R₂ to R₆, L₁, L₂, n, m, p, a₁ and a₂ may be the same as those described above.

In Chemical Formulae 1-7 to 1-9, R₁₅ to R₁₉ may each independently be hydrogen, deuterium, halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₁-C₆₀ heterocycloalkenyl group, a substituted or unsubstituted C₆-C₆₀ aryl group, a substituted or unsubstituted C₆-C₆₀ alkyl aryl group, a substituted or unsubstituted C₆-C₆₀ aryl alkyl group, a substituted or unsubstituted C₂-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₆-C₆₀ aryloxy group, a substituted or unsubstituted C₆-C₆₀ arylthio group, a substituted or unsubstituted C₈-C₆₀ condensed polycyclic group, —SiRR′R″, —P(═O) RR′, —NRR′, —BRR′, —C(═O) R or —S(═O)₂R.

Two or more adjacent ones of R₁₅ to R₁₉ may optionally be combined with each other to form a substituted or unsubstituted C₃-C₆₀ cycloalkyl ring, a substituted or unsubstituted C₅-C₆₀ cycloalkenyl ring, a substituted or unsubstituted C₃-C₆₀ heterocycloalkyl ring, a substituted or unsubstituted C₁-C₆₀ heterocycloalkenyl ring, a substituted or unsubstituted C₆-C₆₀ aryl ring, or a substituted or unsubstituted C₂-C₆₀ heteroaryl ring.

R, R′ and R″ may each independently be hydrogen, deuterium, halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₁-C₆₀ heterocycloalkenyl group, a substituted or unsubstituted C₆-C₆₀ aryl group, a substituted or unsubstituted C₆-C₆₀ alkyl aryl group, a substituted or unsubstituted C₆-C₆₀ aryl alkyl group, a substituted or unsubstituted C₂-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₆-C₆₀ aryloxy group, a substituted or unsubstituted C₆-C₆₀ arylthio group, or a substituted or unsubstituted C₈-C₆₀ condensed polycyclic group.

For example, R₁₅ to R₁₉ may each independently be hydrogen, deuterium, a substituted or unsubstituted C₁-C₁₀ alkyl group, a substituted or unsubstituted C₆-C₂₀ aryl group, a substituted or unsubstituted C₆-C₂₀ alkyl aryl group, or a substituted or unsubstituted C₆-C₂₀ aryl alkyl group.

For example, R₁₅ to R₁₉ may each independently be hydrogen, deuterium, a deuterium-substituted or unsubstituted C₁-C₁₀ alkyl group, a deuterium-substituted or unsubstituted C₆-C₂₀ aryl group, a deuterium-substituted or unsubstituted C₆-C₆₀ alkyl aryl group, or a deuterium-substituted or unsubstituted C₆-C₆₀ aryl alkyl group.

In Chemical Formula 1-7, w and y may each independently be an integer of 0 to 4. For example, w and y may each independently be 0 or 1.

When w and y are each independently 2 or more, a plurality of each of R₁₅ and R₁₆ may be the same as or different from each other. For example, when y is 2 or more, a plurality of R₁₅ may be the same as or different from each other. For example, when w is 2 or more, a plurality of R₁₆ may be the same as or different from each other.

In Chemical Formula 1-8 and Chemical Formula 1-9, L₃ may be a direct bond, a substituted or unsubstituted C₆-C₆₀ arylene group, or a substituted or unsubstituted C₂-C₆₀ heteroarylene group. For example, L₃ may be a direct bond, a substituted or unsubstituted C₆-C₄₀ arylene group, or a substituted or unsubstituted C₂-C₂₀ heteroarylene group. L₃ may be a direct bond, a substituted or unsubstituted C₆-C₂₀ arylene group, or a substituted or unsubstituted C₂-C₂₀ heteroarylene group.

For example, L₃ may be a direct bond, a deuterium-substituted or unsubstituted C₆-C₆₀ arylene group, or a deuterium-substituted or unsubstituted C₂-C₆₀ heteroarylene group. For example, L₃ may be a direct bond, a deuterium-substituted or unsubstituted C₆-C₄₀ arylene group, or a deuterium-substituted or unsubstituted C₂-C₂₀ heteroarylene group. L₃ may be a direct bond, a deuterium-substituted or unsubstituted C₆-C₂₀ arylene group, or a deuterium-substituted or unsubstituted C₂-C₂₀ heteroarylene group.

For example, L₃ may be a divalent group in which any one hydrogen is removed from any one selected from the group consisting of a substituted or unsubstituted phenyl group, a substituted or unsubstituted naphthalene group, a substituted or unsubstituted anthracene group, a substituted or unsubstituted pyridine group, a substituted or unsubstituted pyridazine group, a substituted or unsubstituted pyrazine group, a substituted or unsubstituted triazine group, a substituted or unsubstituted indene group, a substituted or unsubstituted dihydroindene ring, a substituted or unsubstituted fluorene group, a substituted or unsubstituted spirobifluorene group, a substituted or unsubstituted indoline group, a substituted or unsubstituted indole group, a substituted or unsubstituted isoindole group, a substituted or unsubstituted indolizine group, a substituted or unsubstituted indazole group, a substituted or unsubstituted benzimidazole group, a substituted or unsubstituted azaindole group, a substituted or unsubstituted benzofuran group, a substituted or unsubstituted isobenzofuran group, a substituted or unsubstituted benzothiophene group, a substituted or unsubstituted tetrahydroquinoline group, a substituted or unsubstituted dihydroquinoline group, a substituted or unsubstituted quinoline group, a substituted or unsubstituted isoquinoline group, and a substituted or unsubstituted quinoxaline group.

For example, L₃ may be a divalent group in which any one hydrogen is removed from any one selected from the group consisting of a deuterium-substituted or unsubstituted phenyl group, a deuterium-substituted or unsubstituted naphthalene group, a deuterium-substituted or unsubstituted anthracene group, a deuterium-substituted or unsubstituted pyridine group, a deuterium-substituted or unsubstituted pyridazine group, a deuterium-substituted or unsubstituted pyrazine group, a deuterium-substituted or unsubstituted triazine group, a deuterium-substituted or unsubstituted indene group, a deuterium-substituted or unsubstituted dihydroindene, a deuterium-substituted or unsubstituted fluorene group, a deuterium-substituted or unsubstituted spirobifluorene group, a deuterium-substituted or unsubstituted indoline group, a deuterium-substituted or unsubstituted indole group, a deuterium-substituted or unsubstituted isoindole group, a deuterium-substituted or unsubstituted indolizine group, a deuterium-substituted or unsubstituted indazole group, a deuterium-substituted or unsubstituted benzimidazole group, a deuterium-substituted or unsubstituted azaindole group, a deuterium-substituted or unsubstituted benzofuran group, a deuterium-substituted or unsubstituted isobenzofuran group, a deuterium-substituted or unsubstituted benzothiophene group, a deuterium-substituted or unsubstituted tetrahydroquinoline group, a deuterium-substituted or unsubstituted dihydroquinoline group, a deuterium-substituted or unsubstituted quinoline group, a deuterium-substituted or unsubstituted isoquinoline group, and a deuterium-substituted or unsubstituted quinoxaline group.

In Chemical Formula 1-8 and Chemical Formula 1-9, a₃ may be an integer of 0 to 4. For example, a₃ may be 0, 1, or 2. When a₃ is 2 or more, multiple L₃ may be the same as or different from each other.

In Chemical Formula 1-8 and Chemical Formula 1-9, L₄ may be a trivalent organic group in which any two hydrogens are removed from a substituted or unsubstituted C₆-C₆₀ aryl group, a substituted or unsubstituted C₆-C₆₀ alkyl aryl group, a substituted or unsubstituted C₆-C₆₀ aryl alkyl group, a substituted or unsubstituted C₂-C₆₀ heteroaryl group, a substituted or unsubstituted C₂-C₆₀ alkyl heteroaryl group, or a substituted or unsubstituted C₂-C₆₀ heteroaryl alkyl group. For example, L₄ may be a trivalent organic group in which any two hydrogens are removed from a substituted or unsubstituted C₆-C₂₀ aryl group, a substituted or unsubstituted C₆-C₂₀ alkyl aryl group, a substituted or unsubstituted C₆-C₂₀ aryl alkyl group, or a substituted or unsubstituted C₂-C₂₀ heteroaryl group, a substituted or unsubstituted C₂-C₂₀ alkyl heteroaryl group, or a substituted or unsubstituted C₂-C₂₀ heteroaryl alkyl group.

For example, L₄ may be a trivalent organic group in which any two hydrogens are removed from a deuterium-substituted or unsubstituted C₆-C₆₀ aryl group, a deuterium-substituted or unsubstituted C₆-C₆₀ alkyl aryl group, a deuterium-substituted or unsubstituted C₆-C₆₀ aryl alkyl group, a deuterium-substituted or unsubstituted C₂-C₆₀ heteroaryl group, a deuterium-substituted or unsubstituted C₂-C₆₀ alkyl heteroaryl group, or a deuterium-substituted or unsubstituted C₂-C₆₀ heteroaryl alkyl group. For example, L₄ may be a trivalent organic group in which any two hydrogens are removed from a deuterium-substituted or unsubstituted C₆-C₂₀ aryl group, a deuterium-substituted or unsubstituted C₆-C₂₀ alkyl aryl group, a deuterium substituted or unsubstituted C₆-C₂₀ aryl alkyl group, a deuterium-substituted or unsubstituted C₂-C₂₀ heteroaryl group, a deuterium-substituted or unsubstituted C₂-C₂₀ alkyl heteroaryl group, or a deuterium-substituted or unsubstituted C₂-C₂₀ heteroaryl alkyl group.

For example, L₄ may be a trivalent group in which any two hydrogens are removed from any one selected from the group consisting of a substituted or unsubstituted phenyl group, a substituted or unsubstituted naphthalene group, a substituted or unsubstituted anthracene group, a substituted or unsubstituted pyridine group, a substituted or unsubstituted pyridazine group, a substituted or unsubstituted pyrazine group, a substituted or unsubstituted triazine group, a substituted or unsubstituted indene group, a substituted or unsubstituted dihydroindene ring, a substituted or unsubstituted fluorene group, a substituted or unsubstituted spirobifluorene group, a substituted or unsubstituted indoline group, a substituted or unsubstituted indole group, a substituted or unsubstituted isoindole group, a substituted or unsubstituted indolizine group, a substituted or unsubstituted indazole group, a substituted or unsubstituted benzimidazole group, a substituted or unsubstituted azaindole group, a substituted or unsubstituted benzofuran group, a substituted or unsubstituted isobenzofuran group, a substituted or unsubstituted benzothiophene group, a substituted or unsubstituted tetrahydroquinoline group, a substituted or unsubstituted dihydroquinoline group, a substituted or unsubstituted quinoline group, a substituted or unsubstituted isoquinoline group, and a substituted or unsubstituted quinoxaline group.

For example, L₄ may be a trivalent group in which any two hydrogens are removed from any one selected from the group consisting of a deuterium-substituted or unsubstituted phenyl group, a deuterium-substituted or unsubstituted naphthalene group, a deuterium-substituted or unsubstituted anthracene group, a deuterium-substituted or unsubstituted pyridine group, a deuterium-substituted or unsubstituted pyridazine group, a deuterium-substituted or unsubstituted pyrazine group, a deuterium-substituted or unsubstituted triazine group, a deuterium-substituted or unsubstituted indene group, a deuterium-substituted or unsubstituted dihydroindene, a deuterium-substituted or unsubstituted fluorene group, a deuterium-substituted or unsubstituted spirobifluorene group, a deuterium-substituted or unsubstituted indoline group, a deuterium-substituted or unsubstituted indole group, a deuterium-substituted or unsubstituted isoindole group, a deuterium-substituted or unsubstituted indolizine group, a deuterium-substituted or unsubstituted indazole group, a deuterium-substituted or unsubstituted benzimidazole group, a deuterium-substituted or unsubstituted azaindole group, a deuterium-substituted or unsubstituted benzofuran group, a deuterium-substituted or unsubstituted isobenzofuran group, a deuterium-substituted or unsubstituted benzothiophene group, a deuterium-substituted or unsubstituted tetrahydroquinoline group, a deuterium-substituted or unsubstituted dihydroquinoline group, a deuterium-substituted or unsubstituted quinoline group, a deuterium-substituted or unsubstituted isoquinoline group, and a deuterium-substituted or unsubstituted quinoxaline group.

In Chemical Formula 1-9, two of each of R₁₇ to R₁₉ may be the same as or different from each other.

A degree of deuterium substitution of the heterocyclic compound according to embodiments may be in a range from 0% to 100%. The “degree of deuterium substitution” may be a value calculated as a percentage of the number of deuterium atoms relative to a total sum of the number of hydrogen atoms and the number of deuterium atoms included in the heterocyclic compound. For example, the degree of deuterium substitution of benzene substituted with five deuterium atoms may be about 83.33%.

The degree of deuterium substitution of the heterocyclic compound according to some embodiments may be in a range from 1% to 100%, from 5% to 100%, from 20% to 100%, from 30% to 100%, from 40% to 100%, or from 50% to 100%.

The degree of deuterium substitution of the heterocyclic compound according to some embodiments may be in a range from 0% to 90%, from 0% to 80%, from 0% to 70%, from 0% to 60%, or from 0% to 50%.

The heterocyclic compound according to embodiments may include at least one of compounds represented by chemical formulae below.

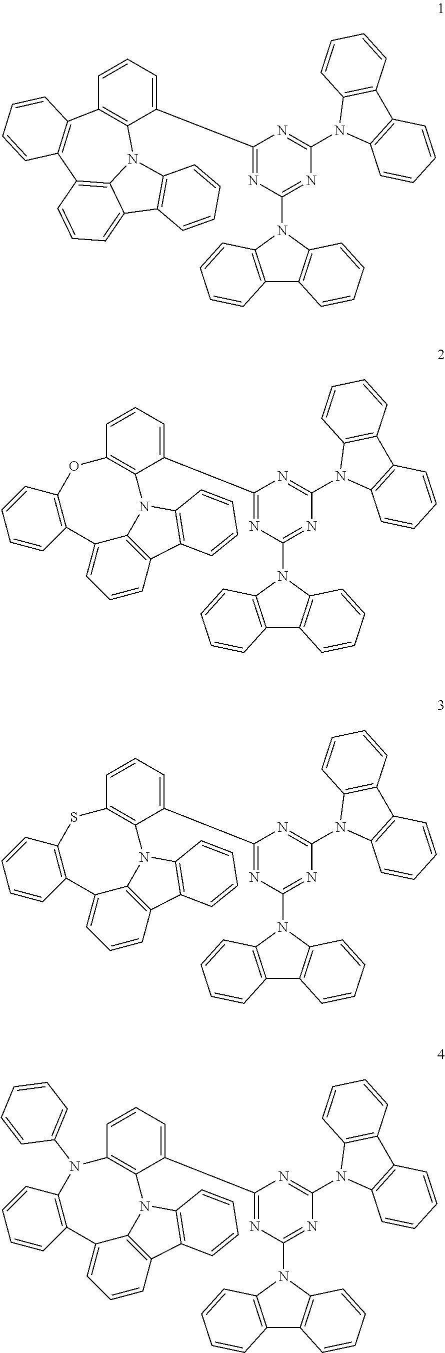 [SMILES: c1ccc2c(c1)-c1cccc(-c3nc(-n4c5ccccc5c5ccccc54)nc(-n4c5ccccc5c5ccccc54)n3)c1-n1c3ccccc3c3cccc-2c31 ; c1ccc2c(c1)oc1cccc(-c3nc(-n4c5ccccc5c5ccccc54)nc(-n4c5ccccc5c5ccccc54)n3)c1n1c3ccccc3c3cccc2c31 ; c1ccc2c(c1)sc1cccc(-c3nc(-n4c5ccccc5c5ccccc54)nc(-n4c5ccccc5c5ccccc54)n3)c1n1c3ccccc3c3cccc2c31 ; c1ccc(-n2c3ccccc3c3cccc4c5ccccc5n(c5c(-c6nc(-n7c8ccccc8c8ccccc87)nc(-n7c8ccccc8c8ccccc87)n6)cccc52)c34)cc1]

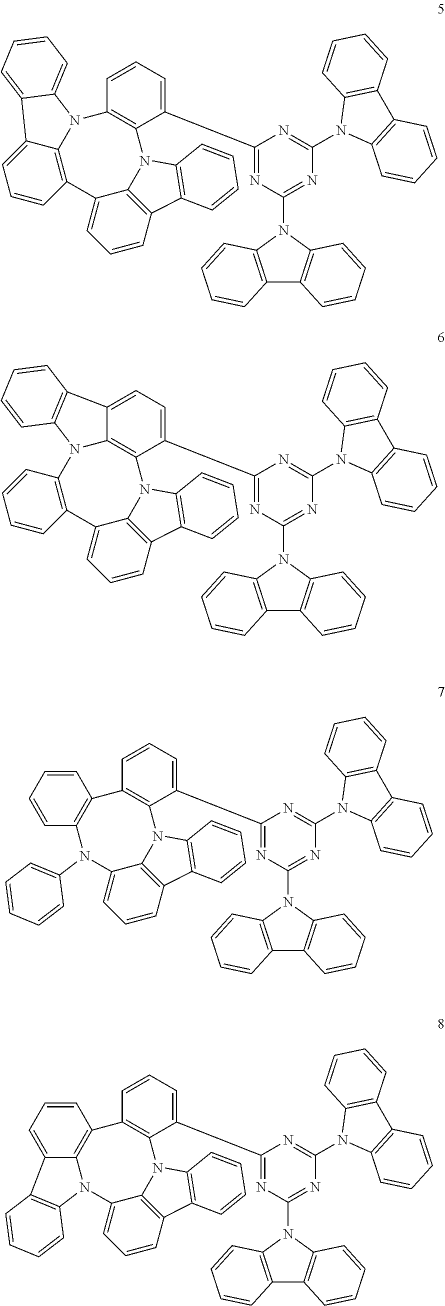 [SMILES: c1ccc2c(c1)c1ccccc1n2-c1nc(-c2cccc3c2n2c4ccccc4c4cccc(c5cccc6c7ccccc7n3c65)c42)nc(-n2c3ccccc3c3ccccc32)n1 ; c1ccc2c(c1)c1ccccc1n2-c1nc(-c2ccc3c4ccccc4n4c5ccccc5c5cccc6c7ccccc7n(c2c34)c56)nc(-n2c3ccccc3c3ccccc32)n1 ; c1ccc(-n2c3ccccc3c3cccc(-c4nc(-n5c6ccccc6c6ccccc65)nc(-n5c6ccccc6c6ccccc65)n4)c3n3c4ccccc4c4cccc2c43)cc1 ; c1ccc2c(c1)c1ccccc1n2-c1nc(-c2cccc3c4cccc5c6ccccc6n(c6cccc7c8ccccc8n(c23)c76)c54)nc(-n2c3ccccc3c3ccccc32)n1]

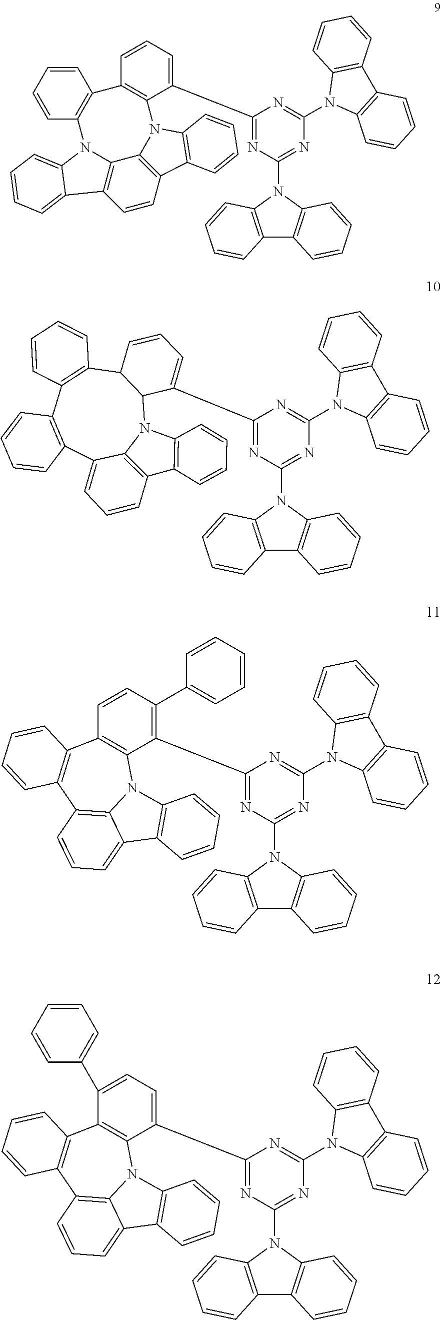 [SMILES: c1ccc2c(c1)c1ccccc1n2-c1nc(-c2cccc3c4ccccc4n4c5ccccc5c5ccc6c7ccccc7n(c23)c6c54)nc(-n2c3ccccc3c3ccccc32)n1 ; C1=CC2c3ccccc3-c3ccccc3-c3cccc4c5ccccc5n(c34)C2C(c2nc(-n3c4ccccc4c4ccccc43)nc(-n3c4ccccc4c4ccccc43)n2)=C1 ; c1ccc(-c2ccc3c(c2-c2nc(-n4c5ccccc5c5ccccc54)nc(-n4c5ccccc5c5ccccc54)n2)-n2c4ccccc4c4cccc(c42)-c2ccccc2-3)cc1 ; c1ccc(-c2ccc(-c3nc(-n4c5ccccc5c5ccccc54)nc(-n4c5ccccc5c5ccccc54)n3)c3c2-c2ccccc2-c2cccc4c5ccccc5n-3c24)cc1]

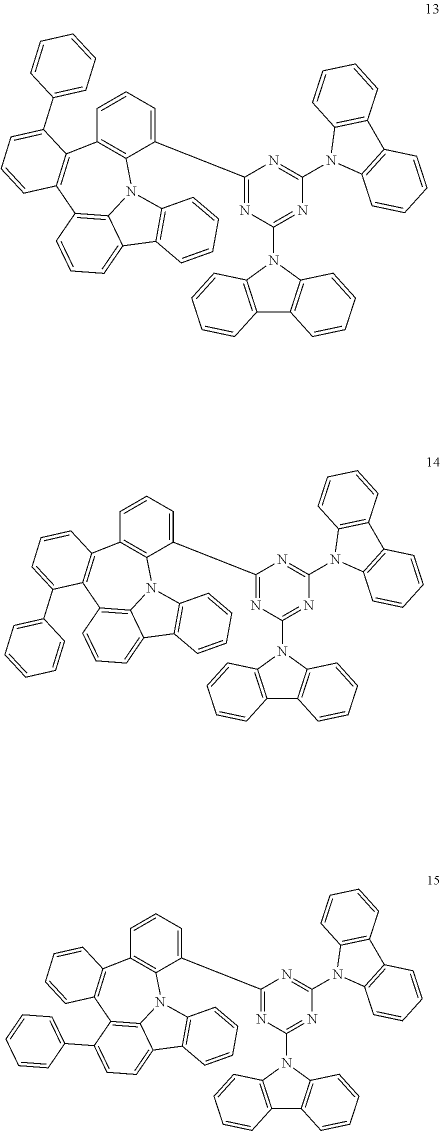 [SMILES: c1ccc(-c2cccc3c2-c2cccc(-c4nc(-n5c6ccccc6c6ccccc65)nc(-n5c6ccccc6c6ccccc65)n4)c2-n2c4ccccc4c4cccc-3c42)cc1 ; c1ccc(-c2cccc3c2-c2cccc4c5ccccc5n(c24)-c2c(-c4nc(-n5c6ccccc6c6ccccc65)nc(-n5c6ccccc6c6ccccc65)n4)cccc2-3)cc1 ; c1ccc(-c2ccc3c4ccccc4n4c3c2-c2ccccc2-c2cccc(-c3nc(-n5c6ccccc6c6ccccc65)nc(-n5c6ccccc6c6ccccc65)n3)c2-4)cc1]

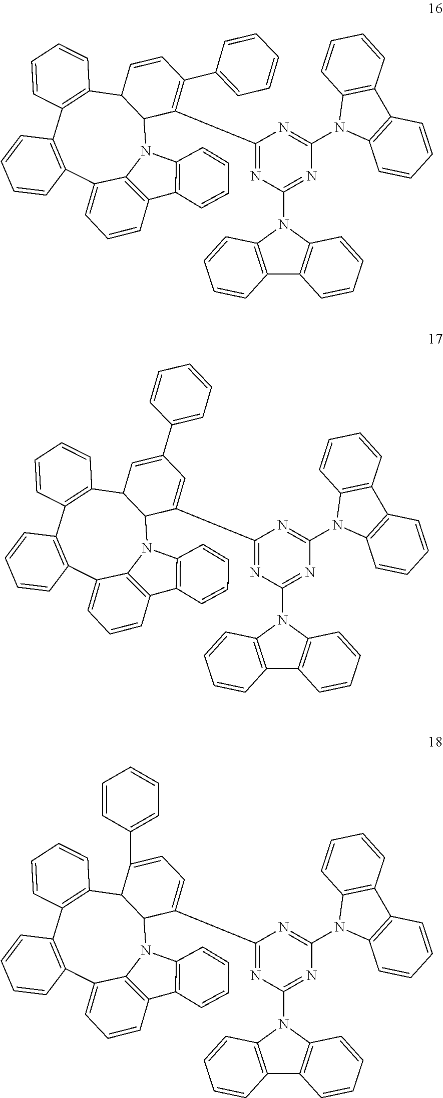 [SMILES: C1=CC2c3ccccc3-c3ccccc3-c3cccc4c5ccccc5n(c34)C2C(c2nc(-n3c4ccccc4c4ccccc43)nc(-n3c4ccccc4c4ccccc43)n2)=C1c1ccccc1 ; C1=C(c2ccccc2)C=C(c2nc(-n3c4ccccc4c4ccccc43)nc(-n3c4ccccc4c4ccccc43)n2)C2C1c1ccccc1-c1ccccc1-c1cccc3c4ccccc4n2c13 ; C1=C(c2ccccc2)C2c3ccccc3-c3ccccc3-c3cccc4c5ccccc5n(c34)C2C(c2nc(-n3c4ccccc4c4ccccc43)nc(-n3c4ccccc4c4ccccc43)n2)=C1]

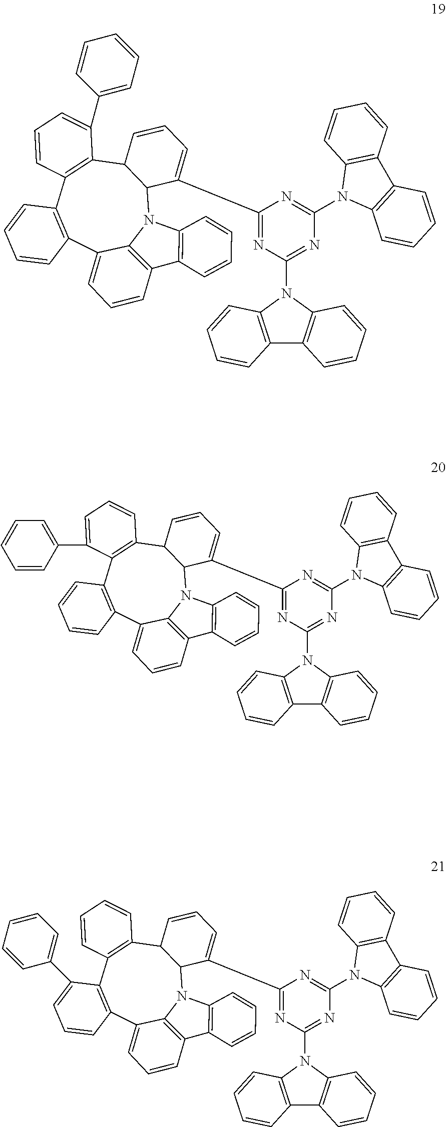 [SMILES: C1=CC2c3c(-c4ccccc4)cccc3-c3ccccc3-c3cccc4c5ccccc5n(c34)C2C(c2nc(-n3c4ccccc4c4ccccc43)nc(-n3c4ccccc4c4ccccc43)n2)=C1 ; C1=CC2c3cccc(-c4ccccc4)c3-c3ccccc3-c3cccc4c5ccccc5n(c34)C2C(c2nc(-n3c4ccccc4c4ccccc43)nc(-n3c4ccccc4c4ccccc43)n2)=C1 ; C1=CC2c3ccccc3-c3c(-c4ccccc4)cccc3-c3cccc4c5ccccc5n(c34)C2C(c2nc(-n3c4ccccc4c4ccccc43)nc(-n3c4ccccc4c4ccccc43)n2)=C1]

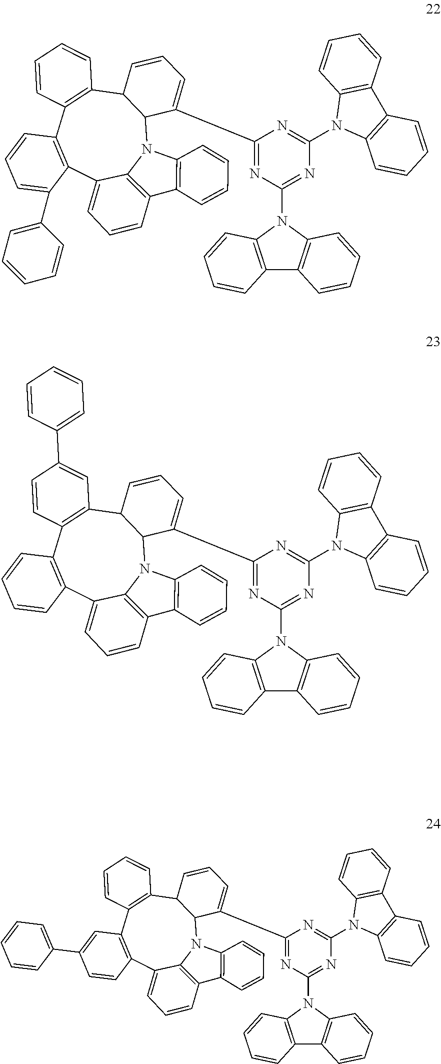 [SMILES: C1=CC2c3ccccc3-c3cccc(-c4ccccc4)c3-c3cccc4c5ccccc5n(c34)C2C(c2nc(-n3c4ccccc4c4ccccc43)nc(-n3c4ccccc4c4ccccc43)n2)=C1 ; C1=CC2c3cc(-c4ccccc4)ccc3-c3ccccc3-c3cccc4c5ccccc5n(c34)C2C(c2nc(-n3c4ccccc4c4ccccc43)nc(-n3c4ccccc4c4ccccc43)n2)=C1 ; C1=CC2c3ccccc3-c3cc(-c4ccccc4)ccc3-c3cccc4c5ccccc5n(c34)C2C(c2nc(-n3c4ccccc4c4ccccc43)nc(-n3c4ccccc4c4ccccc43)n2)=C1]

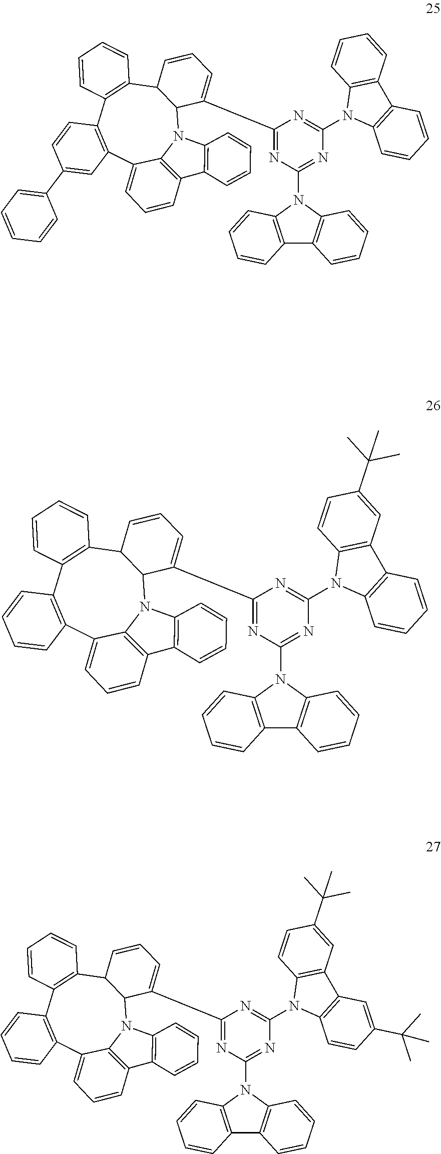 [SMILES: C1=CC2c3ccccc3-c3ccc(-c4ccccc4)cc3-c3cccc4c5ccccc5n(c34)C2C(c2nc(-n3c4ccccc4c4ccccc43)nc(-n3c4ccccc4c4ccccc43)n2)=C1 ; CC(C)(C)c1ccc2c(c1)c1ccccc1n2-c1nc(C2=CC=CC3c4ccccc4-c4ccccc4-c4cccc5c6ccccc6n(c45)C23)nc(-n2c3ccccc3c3ccccc32)n1 ; CC(C)(C)c1ccc2c(c1)c1cc(C(C)(C)C)ccc1n2-c1nc(C2=CC=CC3c4ccccc4-c4ccccc4-c4cccc5c6ccccc6n(c45)C23)nc(-n2c3ccccc3c3ccccc32)n1]

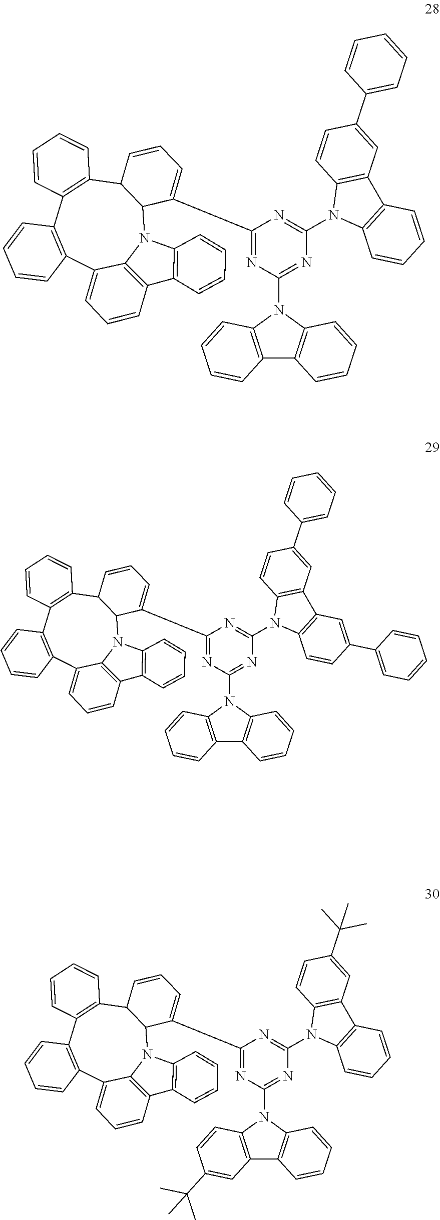 [SMILES: C1=CC2c3ccccc3-c3ccccc3-c3cccc4c5ccccc5n(c34)C2C(c2nc(-n3c4ccccc4c4ccccc43)nc(-n3c4ccccc4c4cc(-c5ccccc5)ccc43)n2)=C1 ; C1=CC2c3ccccc3-c3ccccc3-c3cccc4c5ccccc5n(c34)C2C(c2nc(-n3c4ccccc4c4ccccc43)nc(-n3c4ccc(-c5ccccc5)cc4c4cc(-c5ccccc5)ccc43)n2)=C1 ; CC(C)(C)c1ccc2c(c1)c1ccccc1n2-c1nc(C2=CC=CC3c4ccccc4-c4ccccc4-c4cccc5c6ccccc6n(c45)C23)nc(-n2c3ccccc3c3cc(C(C)(C)C)ccc32)n1]

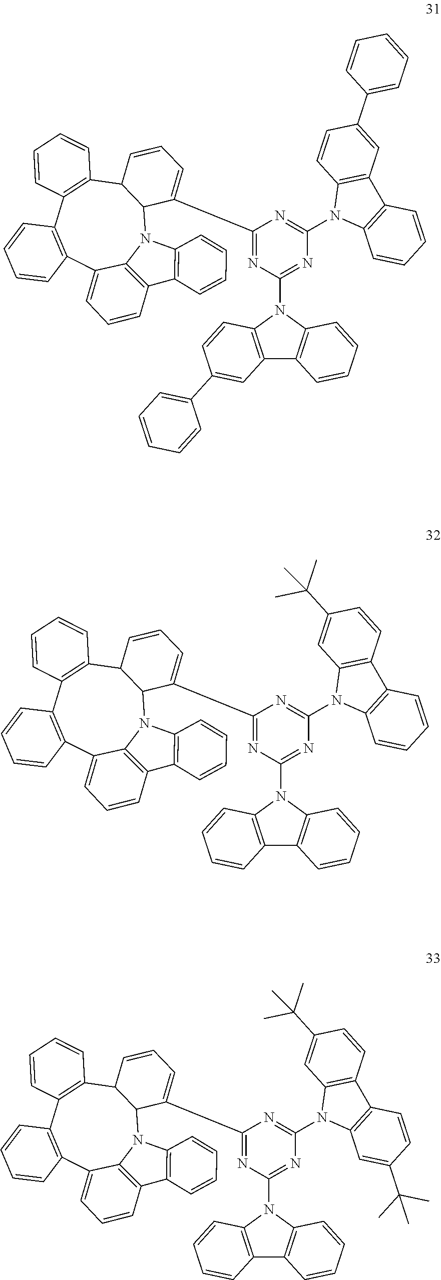 [SMILES: C1=CC2c3ccccc3-c3ccccc3-c3cccc4c5ccccc5n(c34)C2C(c2nc(-n3c4ccccc4c4cc(-c5ccccc5)ccc43)nc(-n3c4ccccc4c4cc(-c5ccccc5)ccc43)n2)=C1 ; CC(C)(C)c1ccc2c3ccccc3n(-c3nc(C4=CC=CC5c6ccccc6-c6ccccc6-c6cccc7c8ccccc8n(c67)C45)nc(-n4c5ccccc5c5ccccc54)n3)c2c1 ; CC(C)(C)c1ccc2c3ccc(C(C)(C)C)cc3n(-c3nc(C4=CC=CC5c6ccccc6-c6ccccc6-c6cccc7c8ccccc8n(c67)C45)nc(-n4c5ccccc5c5ccccc54)n3)c2c1]

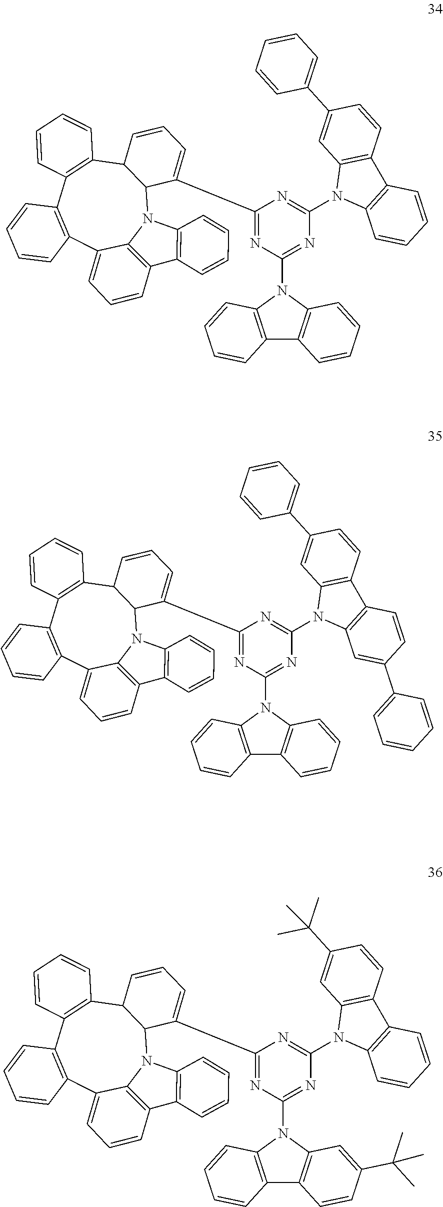 [SMILES: C1=CC2c3ccccc3-c3ccccc3-c3cccc4c5ccccc5n(c34)C2C(c2nc(-n3c4ccccc4c4ccccc43)nc(-n3c4ccccc4c4ccc(-c5ccccc5)cc43)n2)=C1 ; C1=CC2c3ccccc3-c3ccccc3-c3cccc4c5ccccc5n(c34)C2C(c2nc(-n3c4ccccc4c4ccccc43)nc(-n3c4cc(-c5ccccc5)ccc4c4ccc(-c5ccccc5)cc43)n2)=C1 ; CC(C)(C)c1ccc2c3ccccc3n(-c3nc(C4=CC=CC5c6ccccc6-c6ccccc6-c6cccc7c8ccccc8n(c67)C45)nc(-n4c5ccccc5c5ccc(C(C)(C)C)cc54)n3)c2c1]

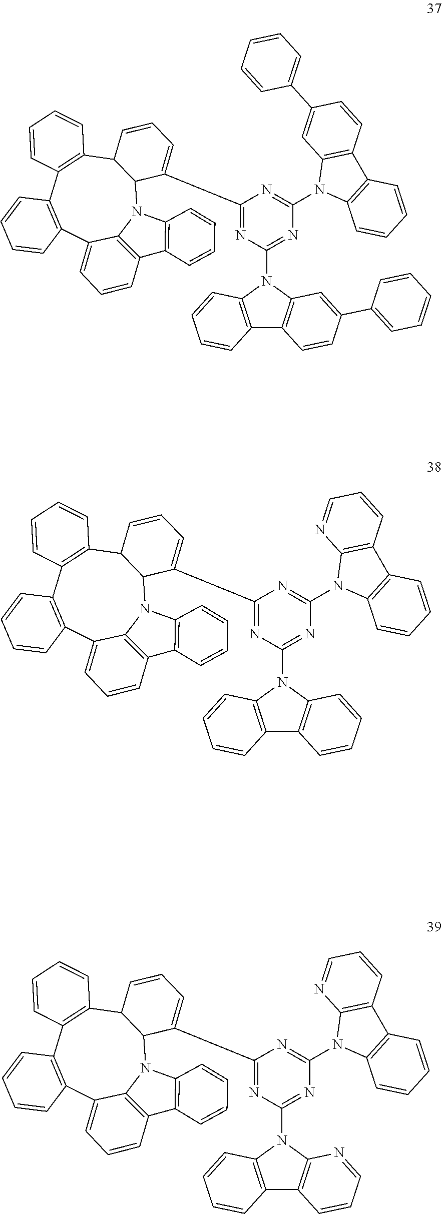 [SMILES: C1=CC2c3ccccc3-c3ccccc3-c3cccc4c5ccccc5n(c34)C2C(c2nc(-n3c4ccccc4c4ccc(-c5ccccc5)cc43)nc(-n3c4ccccc4c4ccc(-c5ccccc5)cc43)n2)=C1 ; C1=CC2c3ccccc3-c3ccccc3-c3cccc4c5ccccc5n(c34)C2C(c2nc(-n3c4ccccc4c4ccccc43)nc(-n3c4ccccc4c4cccnc43)n2)=C1 ; C1=CC2c3ccccc3-c3ccccc3-c3cccc4c5ccccc5n(c34)C2C(c2nc(-n3c4ccccc4c4cccnc43)nc(-n3c4ccccc4c4cccnc43)n2)=C1]

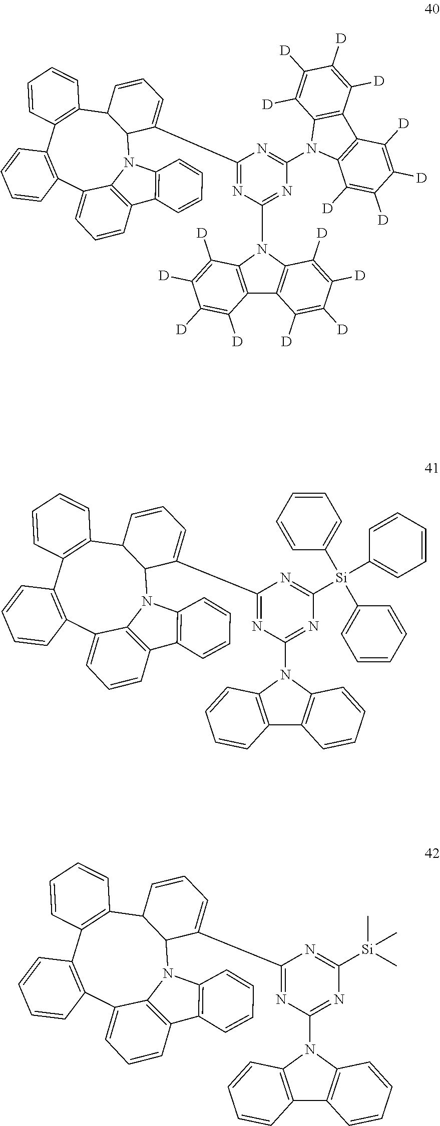 [SMILES: [2H]c1c([2H])c([2H])c2c(c1[2H])c1c([2H])c([2H])c([2H])c([2H])c1n2-c1nc(C2=CC=CC3c4ccccc4-c4ccccc4-c4cccc5c6ccccc6n(c45)C23)nc(-n2c3c([2H])c([2H])c([2H])c([2H])c3c3c([2H])c([2H])c([2H])c([2H])c32)n1 ; C1=CC2c3ccccc3-c3ccccc3-c3cccc4c5ccccc5n(c34)C2C(c2nc(-n3c4ccccc4c4ccccc43)nc([Si](c3ccccc3)(c3ccccc3)c3ccccc3)n2)=C1 ; C[Si](C)(C)c1nc(C2=CC=CC3c4ccccc4-c4ccccc4-c4cccc5c6ccccc6n(c45)C23)nc(-n2c3ccccc3c3ccccc32)n1]

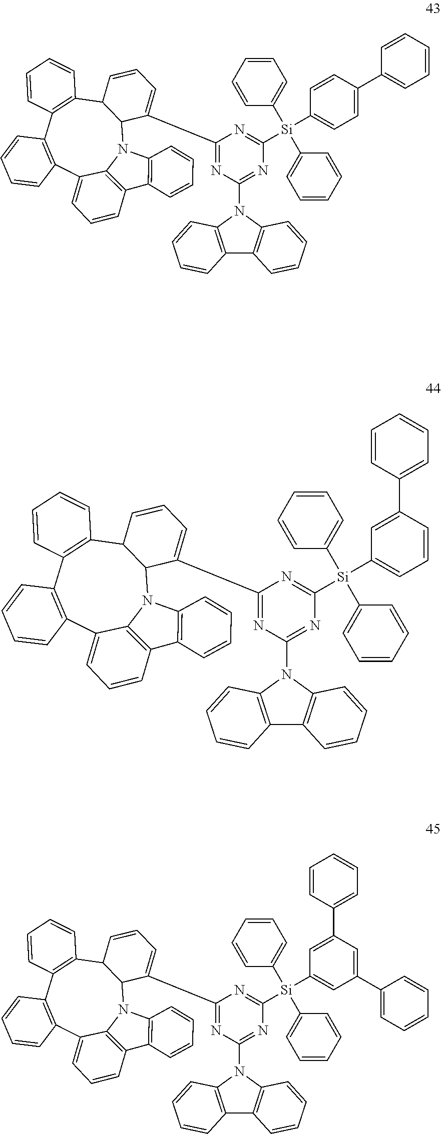 [SMILES: C1=CC2c3ccccc3-c3ccccc3-c3cccc4c5ccccc5n(c34)C2C(c2nc(-n3c4ccccc4c4ccccc43)nc([Si](c3ccccc3)(c3ccccc3)c3ccc(-c4ccccc4)cc3)n2)=C1 ; C1=CC2c3ccccc3-c3ccccc3-c3cccc4c5ccccc5n(c34)C2C(c2nc(-n3c4ccccc4c4ccccc43)nc([Si](c3ccccc3)(c3ccccc3)c3cccc(-c4ccccc4)c3)n2)=C1 ; C1=CC2c3ccccc3-c3ccccc3-c3cccc4c5ccccc5n(c34)C2C(c2nc(-n3c4ccccc4c4ccccc43)nc([Si](c3ccccc3)(c3ccccc3)c3cc(-c4ccccc4)cc(-c4ccccc4)c3)n2)=C1]

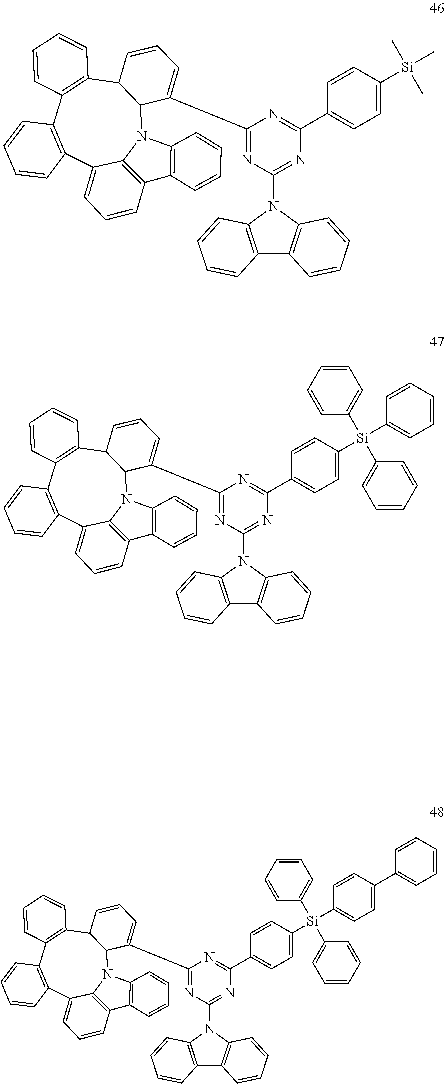 [SMILES: C[Si](C)(C)c1ccc(-c2nc(C3=CC=CC4c5ccccc5-c5ccccc5-c5cccc6c7ccccc7n(c56)C34)nc(-n3c4ccccc4c4ccccc43)n2)cc1 ; C1=CC2c3ccccc3-c3ccccc3-c3cccc4c5ccccc5n(c34)C2C(c2nc(-c3ccc([Si](c4ccccc4)(c4ccccc4)c4ccccc4)cc3)nc(-n3c4ccccc4c4ccccc43)n2)=C1 ; C1=CC2c3ccccc3-c3ccccc3-c3cccc4c5ccccc5n(c34)C2C(c2nc(-c3ccc([Si](c4ccccc4)(c4ccccc4)c4ccc(-c5ccccc5)cc4)cc3)nc(-n3c4ccccc4c4ccccc43)n2)=C1]

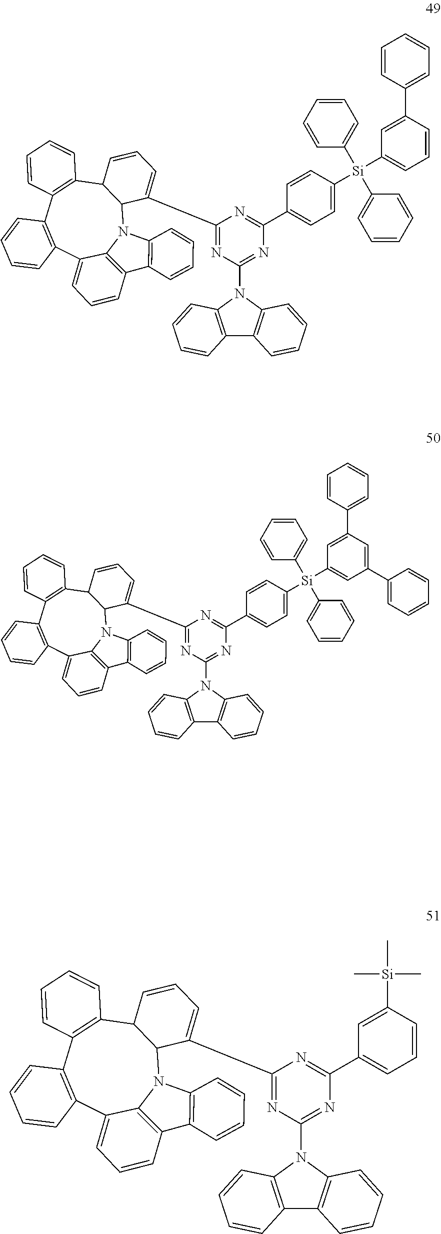 [SMILES: C1=CC2c3ccccc3-c3ccccc3-c3cccc4c5ccccc5n(c34)C2C(c2nc(-c3ccc([Si](c4ccccc4)(c4ccccc4)c4cccc(-c5ccccc5)c4)cc3)nc(-n3c4ccccc4c4ccccc43)n2)=C1 ; C1=CC2c3ccccc3-c3ccccc3-c3cccc4c5ccccc5n(c34)C2C(c2nc(-c3ccc([Si](c4ccccc4)(c4ccccc4)c4cc(-c5ccccc5)cc(-c5ccccc5)c4)cc3)nc(-n3c4ccccc4c4ccccc43)n2)=C1 ; C[Si](C)(C)c1cccc(-c2nc(C3=CC=CC4c5ccccc5-c5ccccc5-c5cccc6c7ccccc7n(c56)C34)nc(-n3c4ccccc4c4ccccc43)n2)c1]

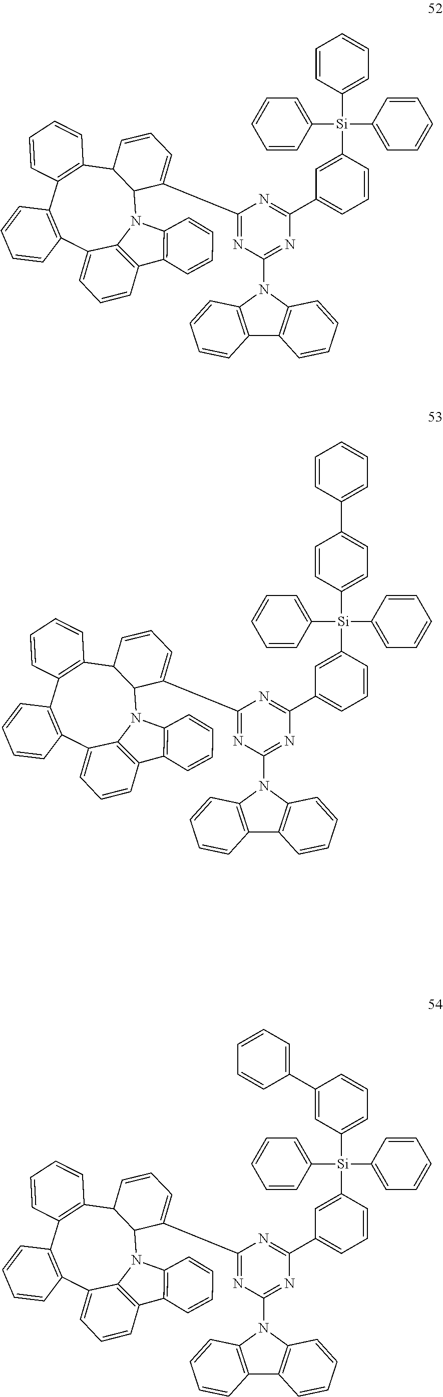 [SMILES: C1=CC2c3ccccc3-c3ccccc3-c3cccc4c5ccccc5n(c34)C2C(c2nc(-c3cccc([Si](c4ccccc4)(c4ccccc4)c4ccccc4)c3)nc(-n3c4ccccc4c4ccccc43)n2)=C1 ; C1=CC2c3ccccc3-c3ccccc3-c3cccc4c5ccccc5n(c34)C2C(c2nc(-c3cccc([Si](c4ccccc4)(c4ccccc4)c4ccc(-c5ccccc5)cc4)c3)nc(-n3c4ccccc4c4ccccc43)n2)=C1 ; C1=CC2c3ccccc3-c3ccccc3-c3cccc4c5ccccc5n(c34)C2C(c2nc(-c3cccc([Si](c4ccccc4)(c4ccccc4)c4cccc(-c5ccccc5)c4)c3)nc(-n3c4ccccc4c4ccccc43)n2)=C1]

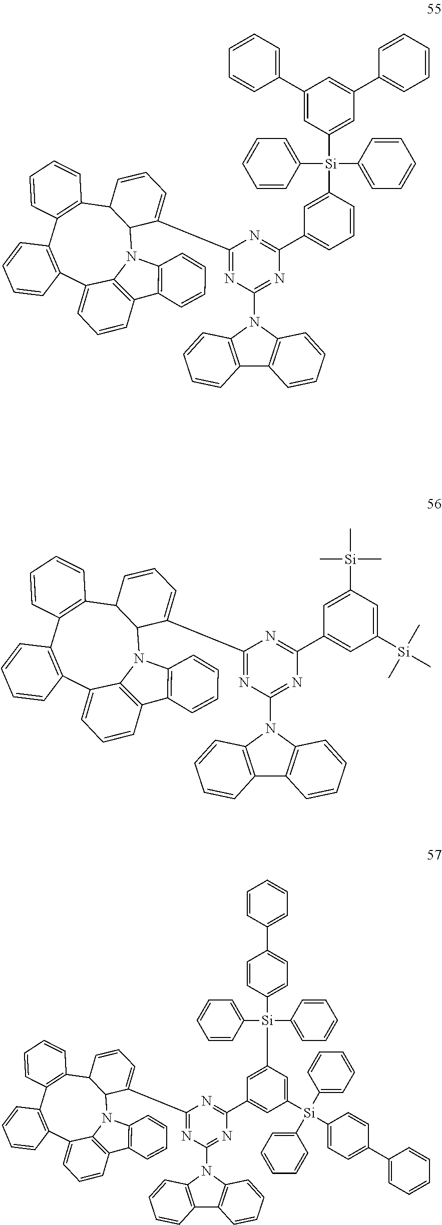 [SMILES: C1=CC2c3ccccc3-c3ccccc3-c3cccc4c5ccccc5n(c34)C2C(c2nc(-c3cccc([Si](c4ccccc4)(c4ccccc4)c4cc(-c5ccccc5)cc(-c5ccccc5)c4)c3)nc(-n3c4ccccc4c4ccccc43)n2)=C1 ; C[Si](C)(C)c1cc(-c2nc(C3=CC=CC4c5ccccc5-c5ccccc5-c5cccc6c7ccccc7n(c56)C34)nc(-n3c4ccccc4c4ccccc43)n2)cc([Si](C)(C)C)c1 ; C1=CC2c3ccccc3-c3ccccc3-c3cccc4c5ccccc5n(c34)C2C(c2nc(-c3cc([Si](c4ccccc4)(c4ccccc4)c4ccc(-c5ccccc5)cc4)cc([Si](c4ccccc4)(c4ccccc4)c4ccc(-c5ccccc5)cc4)c3)nc(-n3c4ccccc4c4ccccc43)n2)=C1]

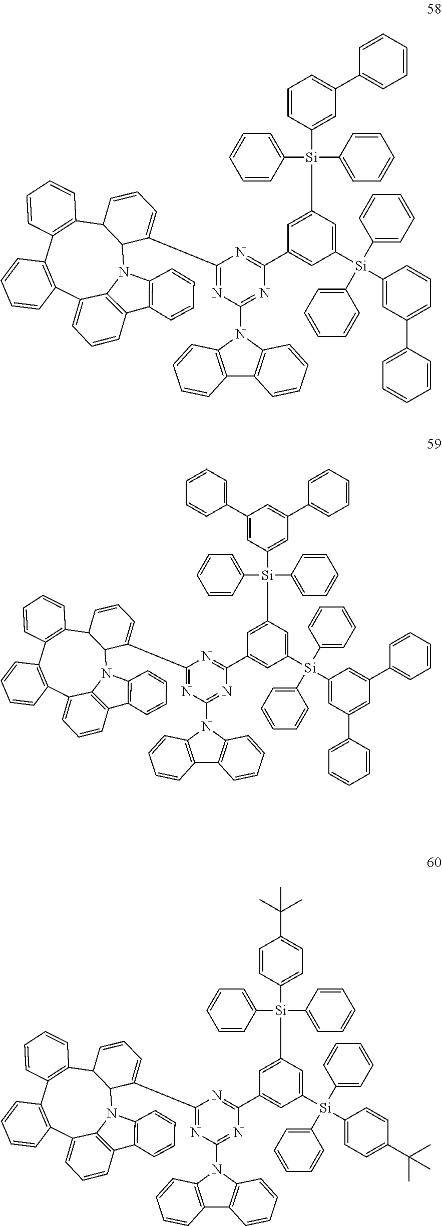

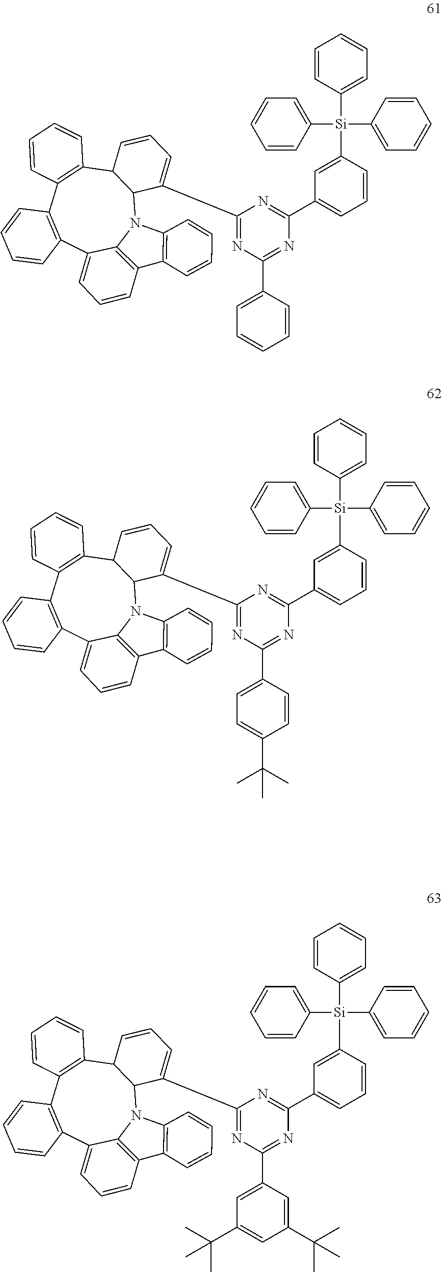 [SMILES: C1=CC2c3ccccc3-c3ccccc3-c3cccc4c5ccccc5n(c34)C2C(c2nc(-c3ccccc3)nc(-c3cccc([Si](c4ccccc4)(c4ccccc4)c4ccccc4)c3)n2)=C1 ; CC(C)(C)c1ccc(-c2nc(C3=CC=CC4c5ccccc5-c5ccccc5-c5cccc6c7ccccc7n(c56)C34)nc(-c3cccc([Si](c4ccccc4)(c4ccccc4)c4ccccc4)c3)n2)cc1 ; CC(C)(C)c1cc(-c2nc(C3=CC=CC4c5ccccc5-c5ccccc5-c5cccc6c7ccccc7n(c56)C34)nc(-c3cccc([Si](c4ccccc4)(c4ccccc4)c4ccccc4)c3)n2)cc(C(C)(C)C)c1]

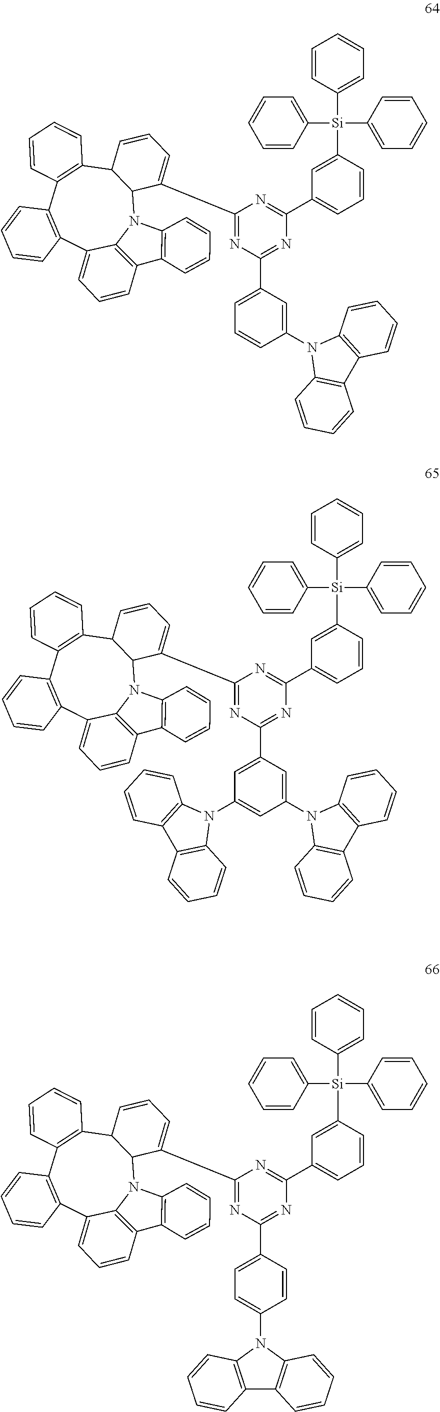 [SMILES: C1=CC2c3ccccc3-c3ccccc3-c3cccc4c5ccccc5n(c34)C2C(c2nc(-c3cccc(-n4c5ccccc5c5ccccc54)c3)nc(-c3cccc([Si](c4ccccc4)(c4ccccc4)c4ccccc4)c3)n2)=C1 ; C1=CC2c3ccccc3-c3ccccc3-c3cccc4c5ccccc5n(c34)C2C(c2nc(-c3cc(-n4c5ccccc5c5ccccc54)cc(-n4c5ccccc5c5ccccc54)c3)nc(-c3cccc([Si](c4ccccc4)(c4ccccc4)c4ccccc4)c3)n2)=C1 ; C1=CC2c3ccccc3-c3ccccc3-c3cccc4c5ccccc5n(c34)C2C(c2nc(-c3ccc(-n4c5ccccc5c5ccccc54)cc3)nc(-c3cccc([Si](c4ccccc4)(c4ccccc4)c4ccccc4)c3)n2)=C1]

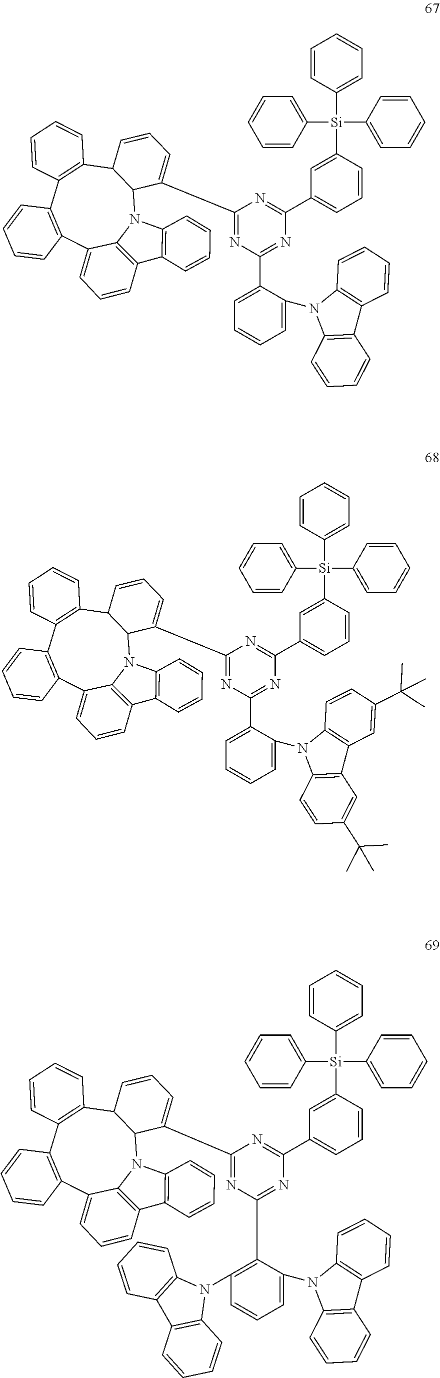 [SMILES: C1=CC2c3ccccc3-c3ccccc3-c3cccc4c5ccccc5n(c34)C2C(c2nc(-c3cccc([Si](c4ccccc4)(c4ccccc4)c4ccccc4)c3)nc(-c3ccccc3-n3c4ccccc4c4ccccc43)n2)=C1 ; CC(C)(C)c1ccc2c(c1)c1cc(C(C)(C)C)ccc1n2-c1ccccc1-c1nc(C2=CC=CC3c4ccccc4-c4ccccc4-c4cccc5c6ccccc6n(c45)C23)nc(-c2cccc([Si](c3ccccc3)(c3ccccc3)c3ccccc3)c2)n1 ; C1=CC2c3ccccc3-c3ccccc3-c3cccc4c5ccccc5n(c34)C2C(c2nc(-c3cccc([Si](c4ccccc4)(c4ccccc4)c4ccccc4)c3)nc(-c3c(-n4c5ccccc5c5ccccc54)cccc3-n3c4ccccc4c4ccccc43)n2)=C1]

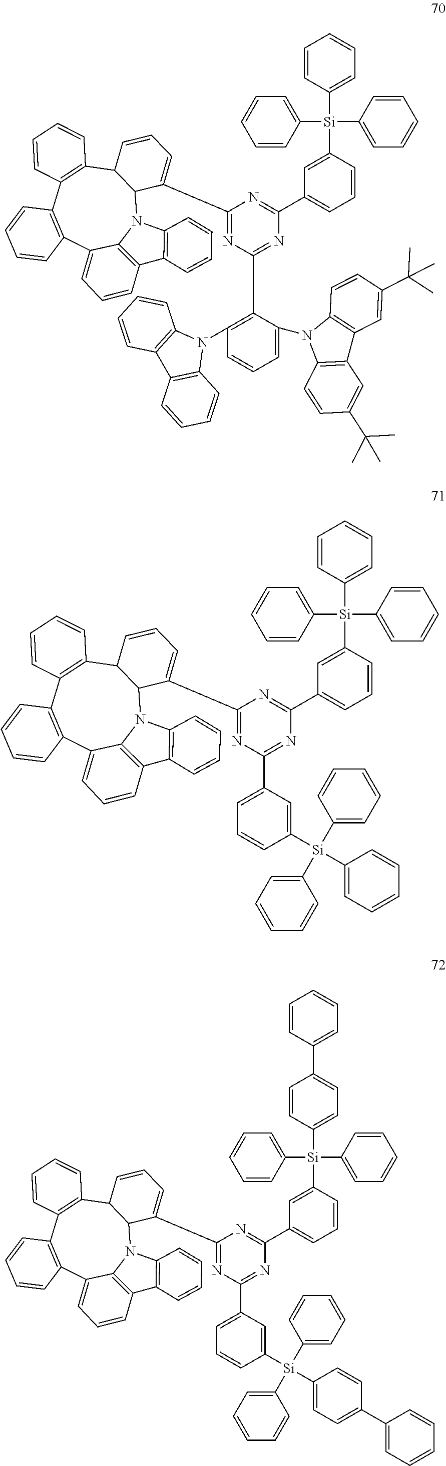

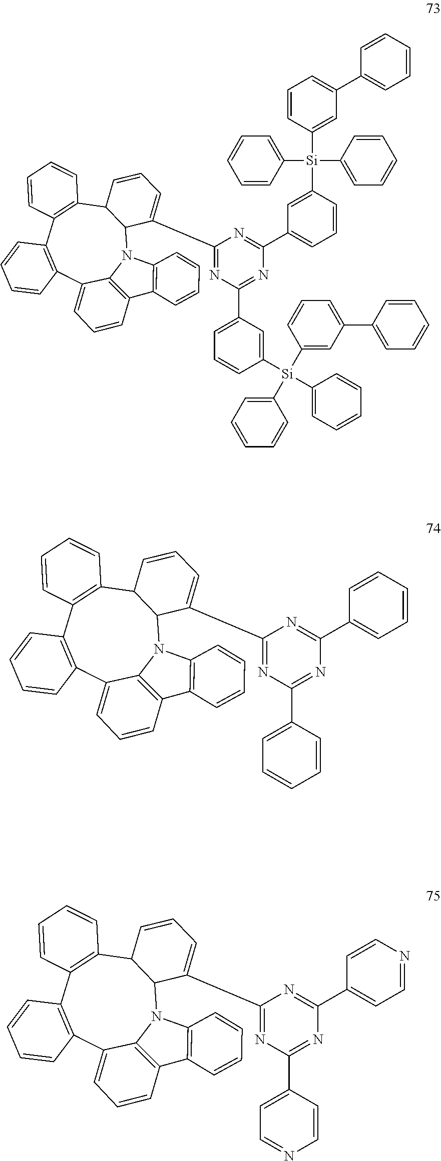 [SMILES: C1=CC2c3ccccc3-c3ccccc3-c3cccc4c5ccccc5n(c34)C2C(c2nc(-c3cccc([Si](c4ccccc4)(c4ccccc4)c4cccc(-c5ccccc5)c4)c3)nc(-c3cccc([Si](c4ccccc4)(c4ccccc4)c4cccc(-c5ccccc5)c4)c3)n2)=C1 ; C1=CC2c3ccccc3-c3ccccc3-c3cccc4c5ccccc5n(c34)C2C(c2nc(-c3ccccc3)nc(-c3ccccc3)n2)=C1 ; C1=CC2c3ccccc3-c3ccccc3-c3cccc4c5ccccc5n(c34)C2C(c2nc(-c3ccncc3)nc(-c3ccncc3)n2)=C1]

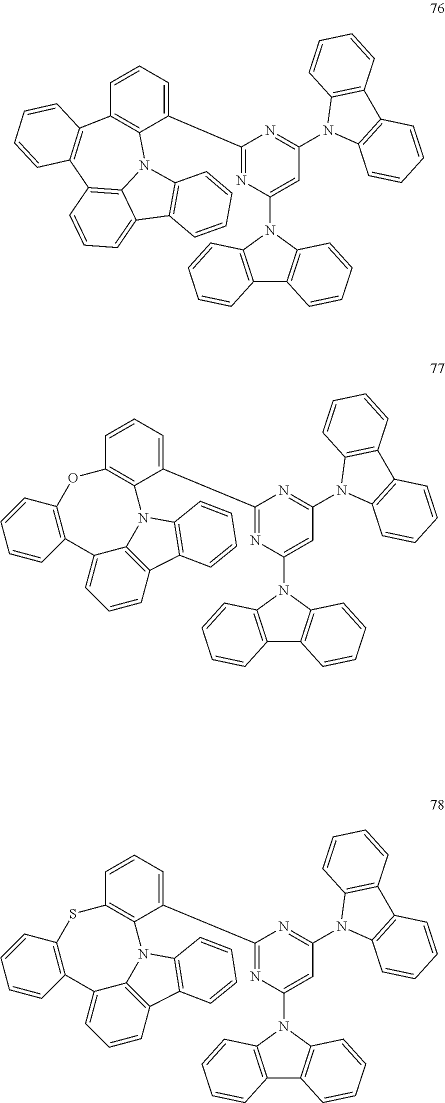 [SMILES: c1ccc2c(c1)-c1cccc(-c3nc(-n4c5ccccc5c5ccccc54)cc(-n4c5ccccc5c5ccccc54)n3)c1-n1c3ccccc3c3cccc-2c31 ; c1ccc2c(c1)oc1cccc(-c3nc(-n4c5ccccc5c5ccccc54)cc(-n4c5ccccc5c5ccccc54)n3)c1n1c3ccccc3c3cccc2c31 ; c1ccc2c(c1)sc1cccc(-c3nc(-n4c5ccccc5c5ccccc54)cc(-n4c5ccccc5c5ccccc54)n3)c1n1c3ccccc3c3cccc2c31]

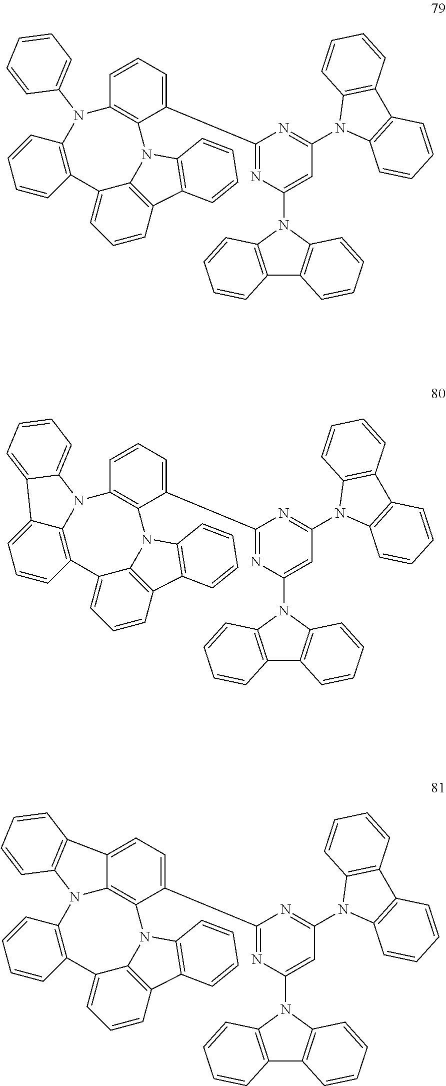 [SMILES: c1ccc(-n2c3ccccc3c3cccc4c5ccccc5n(c5c(-c6nc(-n7c8ccccc8c8ccccc87)cc(-n7c8ccccc8c8ccccc87)n6)cccc52)c34)cc1 ; c1ccc2c(c1)c1ccccc1n2-c1cc(-n2c3ccccc3c3ccccc32)nc(-c2cccc3c2n2c4ccccc4c4cccc(c5cccc6c7ccccc7n3c65)c42)n1 ; c1ccc2c(c1)c1ccccc1n2-c1cc(-n2c3ccccc3c3ccccc32)nc(-c2ccc3c4ccccc4n4c5ccccc5c5cccc6c7ccccc7n(c2c34)c56)n1]

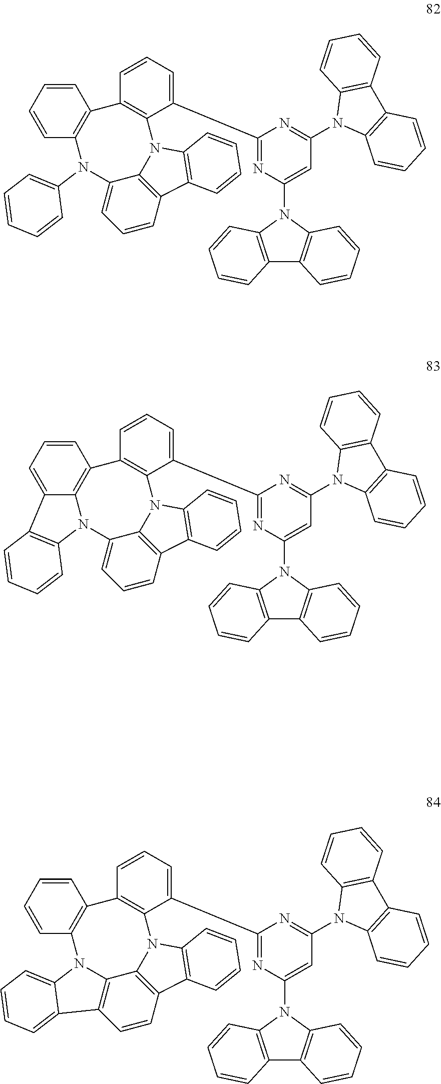 [SMILES: c1ccc(-n2c3ccccc3c3cccc(-c4nc(-n5c6ccccc6c6ccccc65)cc(-n5c6ccccc6c6ccccc65)n4)c3n3c4ccccc4c4cccc2c43)cc1 ; c1ccc2c(c1)c1ccccc1n2-c1cc(-n2c3ccccc3c3ccccc32)nc(-c2cccc3c4cccc5c6ccccc6n(c6cccc7c8ccccc8n(c23)c76)c54)n1 ; c1ccc2c(c1)c1ccccc1n2-c1cc(-n2c3ccccc3c3ccccc32)nc(-c2cccc3c4ccccc4n4c5ccccc5c5ccc6c7ccccc7n(c23)c6c54)n1]

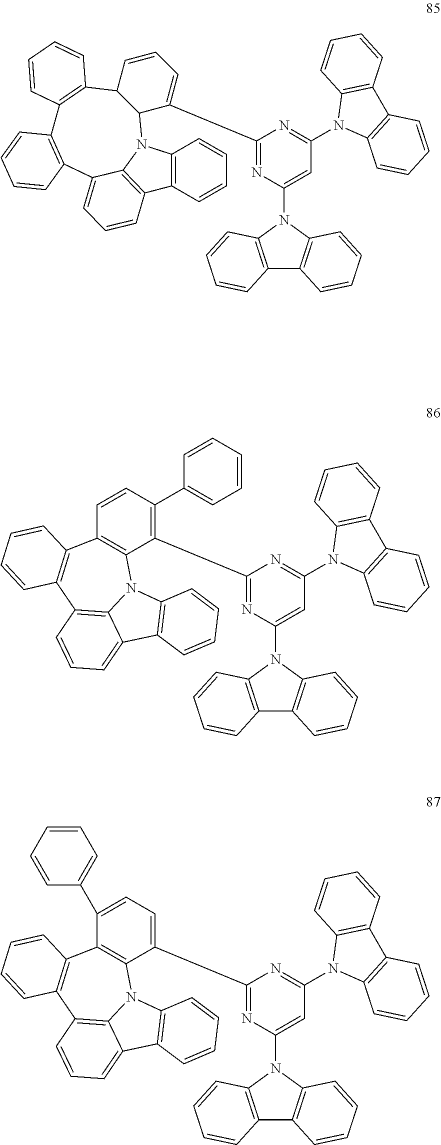 [SMILES: C1=CC2c3ccccc3-c3ccccc3-c3cccc4c5ccccc5n(c34)C2C(c2nc(-n3c4ccccc4c4ccccc43)cc(-n3c4ccccc4c4ccccc43)n2)=C1 ; c1ccc(-c2ccc3c(c2-c2nc(-n4c5ccccc5c5ccccc54)cc(-n4c5ccccc5c5ccccc54)n2)-n2c4ccccc4c4cccc(c42)-c2ccccc2-3)cc1 ; c1ccc(-c2ccc(-c3nc(-n4c5ccccc5c5ccccc54)cc(-n4c5ccccc5c5ccccc54)n3)c3c2-c2ccccc2-c2cccc4c5ccccc5n-3c24)cc1]

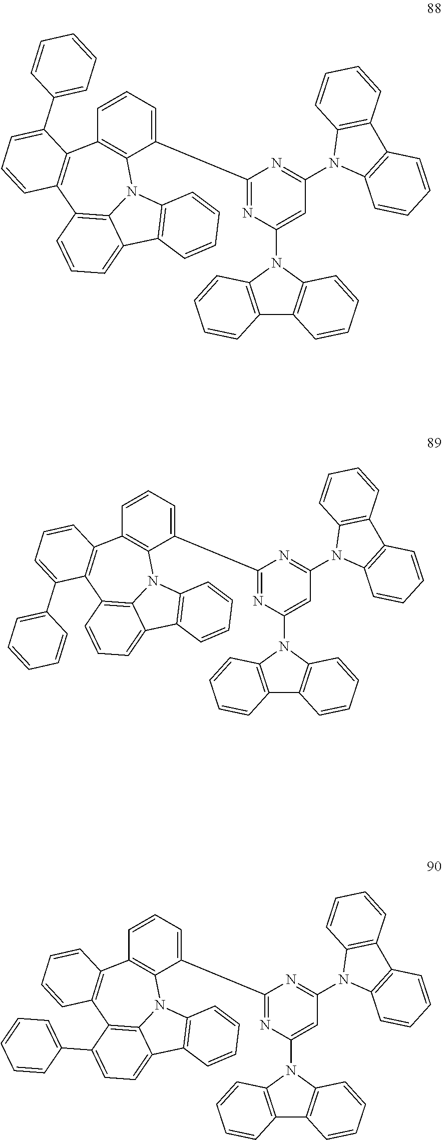 [SMILES: c1ccc(-c2cccc3c2-c2cccc(-c4nc(-n5c6ccccc6c6ccccc65)cc(-n5c6ccccc6c6ccccc65)n4)c2-n2c4ccccc4c4cccc-3c42)cc1 ; c1ccc(-c2cccc3c2-c2cccc4c5ccccc5n(c24)-c2c(-c4nc(-n5c6ccccc6c6ccccc65)cc(-n5c6ccccc6c6ccccc65)n4)cccc2-3)cc1 ; c1ccc(-c2ccc3c4ccccc4n4c3c2-c2ccccc2-c2cccc(-c3nc(-n5c6ccccc6c6ccccc65)cc(-n5c6ccccc6c6ccccc65)n3)c2-4)cc1]

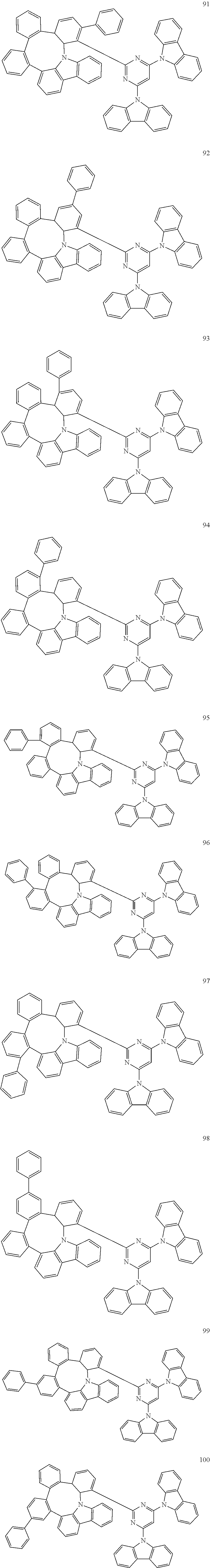

The heterocyclic compound may have a core structure including seven or more rings, and may include an azine-based heteroring bonded to a tertiary nitrogen atom with respect to an ortho position thereof. Accordingly, the heterocyclic compound may have a relatively high triplet energy, may have appropriate hole transport properties, and may achieve a charge balance within an organic device.

Additionally, the heterocyclic compound may have a structure in which a cyclized arylamine structure which is an electron donor implementing HOMO, and an azine-based structure such as pyridine, pyrimidine, triazine, etc., which is an electron acceptor implementing LUMO may be separated and twisted in the molecule. Accordingly, the heterocyclic compound may have improved physical properties as a host for a blue phosphorescent device, and life-span properties and luminous efficiency of a light-emitting device may be improved.

The heterocyclic compound according to the present disclosure may have a stable molecular structure and may have improved chemical stability. Accordingly, the compound structure may be maintained for a long period in the light-emitting device, and the life-span properties of the light-emitting device may be further improved.

In example embodiments, the heterocyclic compound may be provided as a compound for a hole injection layer, a compound for a hole transport layer, a compound for an emission layer, a compound for an auxiliary emission layer, a compound for a hole blocking layer, a compound for an electron transport layer, a compound for an electron injection layer, and/or a compound for a charge generation layer.

In some embodiments, the heterocyclic compound may be provided as a host in an emission layer of the light-emitting device as described below.

According to exemplary embodiments, the heterocyclic compound may be used as a blue light-emitting host.

The heterocyclic compound may have a HOMO level of about-5.6 eV or higher. In some embodiments, the heterocyclic compound may have a HOMO level of about-5.5 eV or higher.

The heterocyclic compound may have a Tl energy level of about 2.8 eV or higher, and thus may be properly used as a host for a blue phosphorescent device.

### Light-Emitting Device

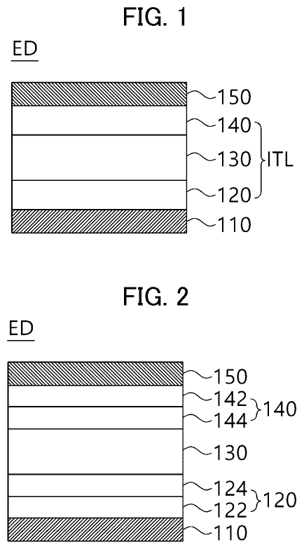

FIGS. 1 to 6 are schematic cross-sectional views illustrating light-emitting devices in accordance with example embodiments.

Referring to FIG. 1, a light-emitting device ED may include a first electrode 110, a second electrode 150, and an intermediate layer ITL disposed between the first electrode 110 and the second electrode 150. The intermediate layer ITL may include an emission layer 130. The intermediate layer ITL may further include an organic layer including a hole transfer region 120 and an electron transfer region 140.

The first electrode 110 may be an anode or a cathode. In some embodiments, the first electrode 110 may serve as an anode, and may serve as a pixel electrode. In this case, the first electrode 110 may include a conductive material with a high work function that promotes hole injection.

In some embodiments, the first electrode 110 may be a transmissive electrode. The first electrode 110 may include a transparent conductive oxide such as indium tin oxide (ITO), indium zinc oxide (IZO), zinc oxide (ZnO), indium tin zinc oxide (ITZO), etc.

In some embodiments, the first electrode 110 may be a translucent electrode or a reflective electrode. The first electrode 110 may include at least one of Ag, Mg, Cu, Al, Pt, Pd, Au, Ni, Nd, Ir, Cr, Li, Ca, Mo, Ti, W, In, Sn, Zn, and an alloy or a compound (e.g., LiF) containing at least one therefrom. For example, the first electrode 110 may include Li, Ca, LiF/Ca (a stacked structure of LiF and Ca), LiF/Al (a stacked structure of LiF and Al), a mixture of Ag and Mg, etc.

The first electrode 110 may have a single-layered structure or a multi-layered structure. For example, the first electrode 110 may have a triple-layered structure of ITO/Ag/ITO.

A thickness of the first electrode 110 may be in a range of about 700 Å to about 10,000 Å. For example, the thickness of the first electrode 110 may be in a range of about 1,000 Å to about 3,000 Å.

The second electrode 150 may be a cathode or an anode. In some embodiments, the second electrode 150 may serve as an electron injection electrode or as a cathode. The second electrode 150 may include a metal, an alloy, an electrically conductive compound, etc., having a low work function.

For example, the second electrode 150 may include lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, etc. The second electrode 150 may include one of the aforementioned materials, or a combination thereof.

The second electrode 150 may be a transmissive electrode, a translucent electrode, or a reflective electrode. The second electrode 150 may have a single-layered structure or a multi-layered structure.

The emission layer 130 may include the heterocyclic compound as described above. In example embodiments, the heterocyclic compound may be provided as a host. In some embodiments, the heterocyclic compound may be provided as a fluorescent host or a host for a phosphorescent device. For example, the heterocyclic compound may be provided as a host for a phosphorescent device or a thermally activated delayed fluorescence (TADF) host.

The emission layer 130 may emit a blue light having a maximum emission central wavelength from about 420 nm to about 490 nm.

In some embodiments, the heterocyclic compound may be included as a blue light-emitting host.

The emission layer 130 may further include a host material different from the heterocyclic compound. For example, the emission layer 130 may include a host material such as an anthracene derivative, a pyrene derivative, a fluoranthene derivative, a chrysene derivative, a dihydrobenzanthracene derivative, a triphenylene derivative, or the like.

In some embodiments, the emission layer 130 may include, e.g., a host material represented by Chemical Formula FH. For example, the compound represented by Chemical Formula FH may be used as a fluorescent host material.

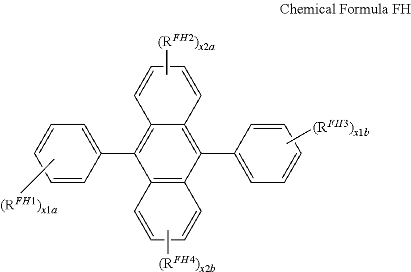

In Chemical Formula FH, R^{FH1} to R^{FH4} may each independently be a hydrogen atom, a deuterium atom, a halogen atom, a substituted or unsubstituted silyl group, a substituted or unsubstituted thio group, a substituted or unsubstituted oxy group, a substituted or unsubstituted C₁-C₁₀ alkyl group, a substituted or unsubstituted C₂-C₁₀ alkenyl group, a substituted or unsubstituted C₆-C₃₀ aryl group, a substituted or unsubstituted C₆-C₃₀ alkyl aryl group, a substituted or unsubstituted C₆-C₃₀ aryl alkyl group, a substituted or unsubstituted C₆-C₃₀ heteroaryl group, a substituted or unsubstituted C₂-C₃₀ alkyl heteroaryl group, a substituted or unsubstituted C₂-C₃₀ heteroaryl alkyl group, or a cyclic group formed through a combination thereof. In some embodiments, in Chemical Formula FH, at least one of R^{FH1} to R^{FH4} may form a condensed ring with a bonded benzene ring.

In Chemical Formula FH, x1a and x1b may each independently be an integer from 0 to 5; and x2a and x2b may each independently be an integer from 0 to 4. When x1a, x1b, x2a, and x2b are each 2 or more, two or more of each of R_{FH1} to R^{FH4} may be the same as or different from each other.

In some embodiments, the emission layer 130 may include, e.g., a host material represented by Chemical Formula PH. For example, the compound represented by Chemical Formula PH may be used as a host material for a phosphorescent device.

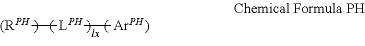

In Chemical Formula PH, RPH may be a substituted or unsubstituted carbazole group. L^{PH} may be a direct linkage, a substituted or unsubstituted C₆-C₃₀ arylene group, or a substituted or unsubstituted C₂-C₃₀ heteroarylene group. Ar^{PH} may be a substituted or unsubstituted C₆-C₃₀ aryl group, a substituted or unsubstituted C₆-C₃₀ alkyl aryl group, or a substituted or unsubstituted C₆-C₃₀ aryl alkyl group, a substituted or unsubstituted C₂-C₃₀ heteroaryl group, a substituted or unsubstituted C₂-C₃₀ alkyl heteroaryl group, or a substituted or unsubstituted C₂-C₃₀ heteroaryl alkyl group.

As described above in the definition of terminology, the term “C₆-C₃₀ aryl group” may encompass a group in which multiple aryl rings are condensed or bonded through a cyclic group (e.g., an alicyclic hydrocarbon ring). For example, a C₆-C₃₀ aryl group may include a fluorenyl group.

As described above in the definition of terminology, the term “C₂-C₃₀ heteroaryl group” may encompass a group in which multiple aryl rings are condensed or bonded through a heterocyclic ring. For example, a C₂-C₃₀ heteroaryl group may include a carbazole group, a dibenzofuran group, a dibenzothiophene group, etc. In some embodiments, a C₂-C₃₀ heteroaryl group may be a group in which multiple aryl rings are condensed or bonded to each other through the same or different heterocyclic rings.

In some embodiments, a substituent included in Ar^{PH} may include a silyl group represented by —Si(R^{sa})(R^{sb})(R^{sc}); and R^{sa}, R^{sb}, and R^{sc} may each independently be hydrogen, halogen, a hydroxyl group, a C₁-C₆₀ alkyl group, a C₁-C₆₀ alkoxy group, a C₁-C₆₀ alkylthio group, a C₆-C₆₀ aryl group, a alkyl aryl group, a C₆-C₆₀ aryl alkyl group, a C₂-C₃₀ heteroaryl group, a C₂-C₆₀ alkyl heteroaryl group, or C₂-C₆₀ heteroaryl alkyl group. At least one of R^{sa}, R^{sb}, and R^{sc} may be a C₆-C₆₀ aryl group or a C₂-C₃₀ heteroaryl group. For example, R^{sa}, R^{sb} and R^{sc} may each independently be a C₆-C₆₀ aryl group or a C₂-C₃₀ heteroaryl group.

In Chemical Formula PH, lx may be an integer from 0 to 10. When lx is 2 or more, two or more of L^{PH} may be the same as or different from each other.

The emission layer 130 may include, e.g., BCPDS (bis(4-(9H-carbazol-9-yl)phenyl)diphenylsilane), POPCPA ((4-(1-(4-(diphenylamino)phenyl) cyclohexyl)phenyl) diphenyl-phosphine oxide), DPEPO (bis[2-(diphenylphosphino)phenyl ether oxide), mCBP (3,3′-di(9H-carbazol-9-yl)-1,1′-biphenyl), CBP (4,4′-bis(N-carbazolyl)-1,1′-biphenyl), mCP (1,3-bis(carbazol-9-yl)benzene), PPF (2,8-bis(diphenylphosphoryl) dibenzo[b,d]furan), TCTA (4,4′,4″-tris(carbazol-9-yl)-triphenylamine), TPBi (1,3,5-tris(1-phenyl-1H-benzo[d]imidazole-2-yl)benzene), Alq3 (tris(8-hydroxyquinolino) aluminum), ADN (9,10-di(naphthalene-2-yl) anthracene), TBADN (2-tert-butyl-9,10-di(naphth-2-yl) anthracene), DSA (distyrylarylene), CDBP (4,4′-bis(9-carbazolyl)-2,2′-dimethyl-biphenyl), MADN (2-methyl-9,10-bis(naphthalen-2-yl) anthracene), CP1 (hexaphenyl cyclotriphosphazene), UGH2 (1,4-bis(triphenylsilyl)benzene), DPSiO3 (hexaphenylcyclotrisiloxane), DPSiO4 (octaphenylcyclotetrasiloxane), or the like, as a host material.

In some embodiments, in the emission layer 130, the host may include one of the materials as described above, or any combination thereof.

The emission layer 130 may further include a dopant interacting with the host material.

In some embodiments, the emission layer 130 may include a dopant represented by Chemical Formula FD. For example, the compound represented by Chemical Formula FD may be used as a fluorescent dopant.

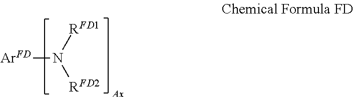

In Chemical Formula FD, Ar^{FD}, R^{FD1}, and R^{FD2} may each independently be a substituted or unsubstituted C₃-C₆₀ carbocyclic group, or a substituted or unsubstituted C₁-C₆₀ heterocyclic group. Ax may be an integer from 1 to 6.

In some embodiments, Ar^{FD} may include a condensed ring structure in which three or more aryl rings or benzene rings are condensed together (e.g., an anthracene group, a chrysene group, a pyrene group, etc.).

In some embodiments, the emission layer 130 may include a phosphorescent dopant. The phosphorescent dopant may include an organometallic compound that includes a central metal and at least one ligand bonded to the central metal via a coordination bond. The central metal may include, e.g., a transition metal, and the ligand may include, e.g., a monodentate ligand, a bidentate ligand, a tridentate ligand, a tetradentate ligand, a pentadentate ligand, a hexadentate ligand, or a combination thereof.

The phosphorescent dopant may include, e.g., a compound represented by Chemical Formula PD.

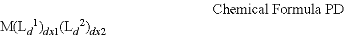

In Chemical Formula PD, M may be a transition metal atom, e.g., iridium (Ir), platinum (Pt), palladium (Pd), osmium (Os), titanium (Ti), gold (Au), hafnium (Hf), europium (Eu), terbium (Tb), rhodium (Rh), rhenium (Re), ruthenium (Ru), copper (Cu), or thulium (Tm).

In Chemical Formula PD, L_{d}¹ may be a ligand represented by Chemical Formula LD1.

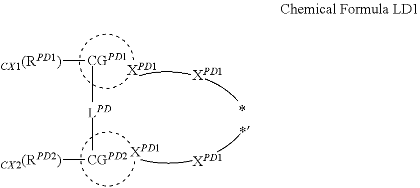

In Chemical Formula LD1, X^{PD1} and X^{PD2} may each independently be C or N.

In some embodiments, one of X^{PD1} and X^{PD2} may be C and the other may be N. In some embodiments, X^{PD1} and X^{PD2} may each be N.

In Chemical Formula LD1, CG^{PD1} and CG^{PD2} may each independently be a substituted or unsubstituted C₃-C₆₀ carbocyclic group, or a substituted or unsubstituted C₁-C₆₀ heterocyclic group. For example, CG^{PD1} and CG^{PD2} may each independently be a pyrrole group, a pyrazole group, an imidazole group, a triazole group, an oxazole group, an isoxazole group, a thiazole group, an isothiazole group, an oxadiazole group or a thiadiazole group, a benzene group, a pyridine group, a pyrimidine group, a naphthalene group, a dibenzofuran group, a dibenzothiophene group, a carbazole group, a fluorene group, a dibenzosilole group, a naphthobenzofuran group, a naphthobenzothiophene group, a benzocarbazole group, a benzofluorene group, a naphthobenzosilole group, a dinaphthofuran group, a dinaphthothiophene group, a dibenzocarbazole group, a dibenzofluorene group, a dinaphthosilole group, an azadibenzofuran group, an azadibenzothiophene group, an azacarbazole group, an azafluorene group, an azadibenzosilole group, an azanaphthobenzofuran group, an azanaphthobenzothiophene group, an azabenzocarbazole group, an azabenzofluorene group, an azanaphthobenzosilole group, an azadinaphthofuran group, an azadinaphthothiophene group, an azadibenzocarbazole group, an azadibenzofluorene group, or an azadinaphthosilole group.

In Chemical Formula LD1, L^{PD} may be a single bond, a substituted or unsubstituted methylene group, a substituted or unsubstituted ethylene group, *—O—**, *—S—**, *—C(═O)—**, *—N(R^{PD3})—**, *—C(R^{PD4})═**, or *═C(R^{PD5})—*′.

In Chemical Formula LD1, X^{PD3} and X^{PD4} may each independently be a chemical bond, O, S, N(R^{PD6}), B(R^{PD7}), P(R^{PD8}), C(R^{PD9})(R^{PD10}), or Si(R^{PD11})(R^{PD12}). The chemical bond may be, e.g., a covalent bond or a coordination bond.

In Chemical Formula LD1, R^{PD1} and R^{PD2} may each independently be hydrogen, deuterium, —F, —Cl, —Br, —I, —SF₅, —OH, —CN, —NO₂, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₁-C₆₀ heterocycloalkenyl group, a substituted or unsubstituted C₆-C₆₀ aryl group, a substituted or unsubstituted C₆-C₆₀ alkyl aryl group, a substituted or unsubstituted C₆-C₆₀ aryl alkyl group, a substituted or unsubstituted C₂-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₆-C₆₀ aryloxy group, a substituted or unsubstituted C₆-C₆₀ arylthio group, a substituted or unsubstituted C₈-C₆₀ condensed polycyclic group, a substituted or unsubstituted silyl group, a substituted or unsubstituted amine group, a substituted or unsubstituted aniline group, —B(R^{PD13})(R^{PD14}), —C(═O)(R^{PD15}), —S(═O)₂(R^{PD16}), or —P(═O)(RP^{D17})(R^{PD18}). The silyl group may be represented by —Si(R^{sa})(R^{sb})(R^{sc}), as explained above.

R^{PD3} to R^{PD18} may each independently be hydrogen, deuterium, —F, —Cl, —Br, —I, —SF₅, —OH, —CN, —NO₂, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₁-C₆₀ heterocycloalkenyl group, a substituted or unsubstituted C₆-C₆₀ aryl group, a substituted or unsubstituted C₆-C₆₀ alkyl aryl group, a substituted or unsubstituted C₆-C₆₀ aryl alkyl group, a substituted or unsubstituted C₂-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₆-C₆₀ aryloxy group, a substituted or unsubstituted C₆-C₆₀ arylthio group, or a substituted or unsubstituted C₈-C₆₀ condensed polycyclic group.

In Chemical Formula LD1, cx1 and cx2 may each independently be an integer from 0 to 10. When at least one of cx1 and cx2 is 2 or more, two or more of R^{PD1} or two or more of R^{PD2} may be the same as or different from each other.

The symbols -* and -*′ each represent a binding site where the ligand represented by Chemical Formula LD1 bonds to M.

In Chemical Formula PD, dx1 may be an integer from 1 to 3. When dx1 is 2 or 3, two or three of L_{d}¹ may be the same as or different from each other. Among two or three of L_{d}¹, CG^{PD1} and/or CG^{PD2} adjacent to each other may be connected to each other through a connecting group such as L^{PD1}, L^{PD2}, etc. The connecting group such as L^{PD1}, L^{PD2}, etc., may each independently be the same as defined in connection with L^{PD}.

In Chemical Formula PD, L_{d}² may be an organic ligand. L_{d}² may include, e.g., a halogen group, CO, NO, CS, picolinate, acetate, oxalate, a diketone group, an isonitrile group, isothiocyanato-N, thiosulphato-S, an alkyl phosphine, phenylphosphine, an aryl phosphine, phosphine oxide, phosphite, or a combination thereof.

In Chemical Formula PD, dx2 is an integer of 0 to 4. When dx2 is 2 or more, two or more of L_{d}² may be the same as or different from each other.

In some embodiments, the emission layer 130 may include a styryl derivative (e.g., 1,4-bis[2-(3-N-ethylcarbazoryl) vinyl]benzene (BCzVB), 4-(di-p-tolylamino)-4′-[(di-p-tolylamino) styryl]stilbene (DPAVB), N-(4-((E)-2-(6-((E)-4-(diphenylamino) styryl) naphthalen-2-yl) vinyl)phenyl)-N-phenylbenzenamine (NBDAVBi), etc.), 4,4′-bis[2-(4-(N,N-diphenylamino)phenyl)vinyl]biphenyl (DPAVBi), perylene or a derivative thereof (e.g., 2,5,8,11-tetra-t-butylperylene (TBP)), pyrene or a derivative thereof (e.g., 1,1-dipyrene, 1,4-dipyrenylbenzene, 1,4-bis(N,N-diphenylamino)pyrene), etc.), etc., as a fluorescent dopant material.

The emission layer 130 may include a metal complex that includes iridium (Ir), platinum (Pt), osmium (Os), gold (Au), titanium (Ti), zirconium (Zr), hafnium (Hf), europium (Eu), terbium (Tb), or thulium (Tm) as a phosphorescent dopant, in addition to the materials described above. For example, FIrpic (iridium (III) bis(4,6-difluorophenylpyridinato-N,C₂′) picolinate), FIr6 (bis(2,4-difluorophenylpyridinato)-tetrakis(1-pyrazolyl) borate iridium (II)), PtOEP (platinum octaethyl porphyrin), etc., may be used as the phosphorescent dopant.

In embodiments, the emission layer 130 may include a boron-containing dopant represented by Chemical Formula BD.

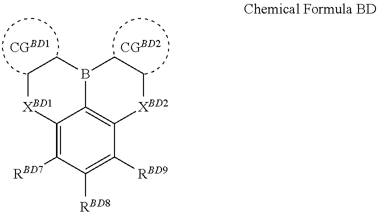

In Chemical Formula BD, X^{BD1} and X^{BD2} may each independently be N(R^{BD1}), P(R^{BD2}), C(R^{BD3})(R^{BD4}), Si(R^{BD5})(R^{BD6}), S or O. In some embodiments, X^{BD1} and X^{BD2} may each be N(R^{BD1}). R^{BD1} to R^{BD6} may each independently be hydrogen, deuterium, a substituted or unsubstituted C₁-C₂₀ alkyl group, a substituted or unsubstituted C₆-C₃₀ aryl group, a substituted or unsubstituted C₆-C₆₀ alkyl aryl group, a substituted or unsubstituted C₆-C₆₀ aryl alkyl group, a substituted or unsubstituted C₂-C₃₀ heteroaryl group, a substituted or unsubstituted C₂-C₃₀ alkyl heteroaryl group, or a substituted or unsubstituted C₂-C₃₀ heteroaryl alkyl group. R^{BD7}, R^{BD8}, and R^{BD9} may each independently be hydrogen, deuterium, halogen, a cyano group, a substituted or unsubstituted amine group, a substituted or unsubstituted boryl group, a substituted or unsubstituted oxy group, a substituted or unsubstituted thio group, a substituted or unsubstituted C₁-C₂₀ alkyl group, a substituted or unsubstituted C₆-C₃₀ aryl group, a substituted or unsubstituted C₆-C₃₀ alkyl aryl group, a substituted or unsubstituted C₆-C₃₀ aryl alkyl group, or a substituted or unsubstituted C₂-C₃₀ heteroaryl group, a substituted or unsubstituted C₂-C₃₀ alkyl heteroaryl group, or a substituted or unsubstituted C₂-C₃₀ heteroaryl alkyl group. R^{BD7}, R^{BD8} and/or R^{BD9} may be bonded to an adjacent group to form a ring.

In Chemical Formula BD, CG^{BD1} and CG^{BD2} represent a cyclic group, and CG^{BD1} and CG^{BD2} may each independently be a substituted or unsubstituted C₃-C₆₀ carbocyclic group, or a substituted or unsubstituted C₁-C₆₀ heterocyclic group. In some embodiments, CG^{BD1} and CG^{BD2} may each independently be a substituted or unsubstituted C₆-C₃₀ aryl group, a substituted or unsubstituted C₆-C₆₀ alkyl aryl group, a substituted or unsubstituted C₆-C₆₀ aryl alkyl group, or a substituted or unsubstituted C₂-C₃₀ heteroaryl group, a substituted or unsubstituted C₂-C₆₀ alkyl heteroaryl group, or a substituted or unsubstituted C₂-C₆₀ heteroaryl alkyl group.

In some embodiments, CG^{BD1} and CG^{BD2} may each independently be a substituted or unsubstituted benzene ring. In this case, the boron-containing dopant may serve as a thermally activated delayed fluorescence (TADF) dopant.

In some embodiments, one of CG^{BD1} and CG^{BD2} may be a non-condensed aryl group or a non-condensed heteroaryl group, and the other one thereof may be a condensed polycyclic aryl group or a condensed polycyclic heteroaryl group. In this case, the boron-containing dopant may serve as a fluorescent dopant.

In some embodiments, the emission layer 130 may include one of the dopant materials as described above, or any combination thereof.

In some embodiments, the emission layer 130 may include two or more host materials. For example, the emission layer 130 may include a hole transporting host and an electron transporting host. In this case, the emission layer 130 may include a hole transporting host, an electron transporting host, a photosensitive agent, and a dopant. In example embodiments, the hole transporting host and the electron transporting host may form an exciplex, and energy may be transferred from the exciplex to the photosensitive agent and from the photosensitive agent to the dopant, thereby inducing a light emission.

Non-limiting examples of the hole transporting host may include a compound represented by Chemical Formula HT as described below. Non-limiting examples of the electron transporting host may include a compound represented by Chemical Formula ET as described below.

In some embodiments, the emission layer 130 may include quantum dots. The quantum dot may include a Group II-VI compound, a Group III-VI compound, a Group I-III-VI compound, a Group III-V group compound, a Group III-II-V group compound, a Group IV-VI compound, a Group IV element, a Group IV compound, or a combination thereof.

The quantum dot may include a core that includes the compound as described above, and a shell surrounding the core. The shell may include an inorganic oxide or a semiconductor compound. Examples of the semiconductor compound may include CdS, CdSe, CdTe, ZnS, ZnSe, ZnTe, ZnSe, ZnTeS, GaAs, GaP, GaSb, HgS, HgSe, HgTe, InAs, InP, InGaP, InSb, AlAs, AlP, AlSb, etc.

In example embodiments, a color of light from a quantum dot may be adjusted according to a particle size of the quantum dot. The quantum dot may be a blue quantum dot, a red quantum dot, or a green quantum dot.

The hole transfer region 120 may be formed between the first electrode 110 and the emission layer 130. The hole transfer region 120 may have a single-layered structure or a multi-layered structure including different materials.

The hole transfer region 120 may include a hole injection layer, a hole transport layer, and/or an electron blocking layer, and may further include an auxiliary emission layer.

In some embodiments, as illustrated in FIG. 2, the hole transfer region 120 may include a hole injection layer 122 and a hole transport layer 124, sequentially stacked from the first electrode 110.

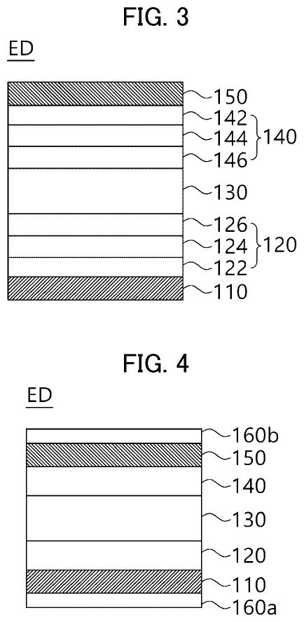

In some embodiments, as illustrated in FIG. 3, the hole transfer region 120 may include a hole injection layer 122, a hole transport layer 124, and an electron blocking layer 126, sequentially stacked from the first electrode 110. The electron blocking layer 126 may block an electron transfer from the electron transfer region 140 to the hole transfer region 120. Accordingly, the generation of excitons in the emission layer 130 may be increased, and light-emission efficiency may be further increased.

For example, the hole transfer region 120 may further include a compound represented by Chemical Formula HT.

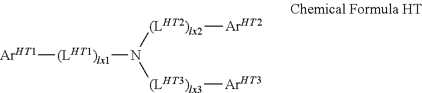

In Chemical Formula HT, L^{HT1}, L^{HT2}, and L^{HT3} may each independently be a direct linkage, a substituted or unsubstituted C₆-C₃₀ arylene group, or a substituted or unsubstituted C₂-C₃₀ heteroarylene group.

In Chemical Formula HT, lx1 to lx3 may each independently be an integer from 0 to 10. When lx1, lx2, or lx3 is 2 or more, two or more of each of L^{HT1}, L^{HT2}, or L^{HT3} may be directly connected by, e.g., carbon atoms (e.g., sp2 carbons) of each aryl ring, to form a substituted or unsubstituted C₆-C₃₀ arylene group, or a substituted or unsubstituted C₂-C₃₀ heteroarylene group.

In Chemical Formula HT, Ar^{HT1} and Ar^{HT2} may each independently be a substituted or unsubstituted C₆-C₃₀ aryl group, a substituted or unsubstituted C₆-C₃₀ alkyl aryl group, a substituted or unsubstituted C₆-C₃₀ aryl alkyl group, or a substituted or unsubstituted C₂-C₃₀ heteroaryl group, a substituted or unsubstituted C₂-C₃₀ alkyl heteroaryl group, or a substituted or unsubstituted C₂-C₃₀ heteroaryl alkyl group. Ar^{HT3} may be a substituted or unsubstituted C₆-C₃₀ aryl group, a substituted or unsubstituted C₆-C₆₀ alkyl aryl group, a substituted or unsubstituted C₆-C₆₀ aryl alkyl group.

In some embodiments, the compound represented by Chemical Formula HT may be a monoamine compound. In some embodiments, the compound represented by Chemical Formula HT may be a diamine compound in which at least one of Ar^{HT1} to Ar^{HT3} includes an amine group as a substituent.

In some embodiments, the compound represented by Chemical Formula HT may be a carbazole-based compound in which at least one of Ar^{HT1} and Ar^{HT2} includes a substituted or unsubstituted carbazole group, or a fluorene-based compound in which at least one of Ar^{HT1} and Ar^{HT2} includes a substituted or unsubstituted fluorene group.

In some embodiments, two adjacent groups among Ar^{HT1} to Ar^{HT3} may be condensed together to form a ring.

For example, the hole transfer region 120 may include m-MTDATA (4,4′,4″-[tris(3-methylphenyl)phenylamino triphenylamine), TDATA (4,4′4″-tris(N,N-diphenylamino)triphenylamine), 2-TNATA (4,4′,4″-tris[N (2-naphthyl)-N-phenylamino-triphenylamine), NPB (N,N′-di(naphthalene-1-yl)-N,N′-diphenyl-benzidine), TPD (N,N′-bis(3-methylphenyl)-N,N′-diphenyl-[1,1′-biphenyl-4,4′-diamine), Spiro-TPD, Spiro-NPB, DNTPD (N¹,N¹′-([1,1′-biphenyl-4,4′-diyl)bis(N¹-phenyl-N⁴,N⁴-di-m-tolylbenzene-1,4-diamine), TAPC (4,4′-cyclohexylidene bis[N,N-bis(4-methylphenyl)benzenamine), HMTPD (4,4′-bis[N,N′-(3-tolyl)amino-3,3′-dimethylbiphenyl), TCTA (4,4′,4″-tris(N-carbazolyl)triphenylamine), PANI/DBSA (polyaniline/dodecylbenzenesulfonic acid), PEDOT/PSS (poly(3,4-ethylenedioxythiophene)/poly(4-styrenesulfonate)), PANI/CSA (polyaniline/Camphor sulfonic acid), PANI/PSS (polyaniline/poly(4-styrenesulfonate)), a phthalocyanine compound, a carbazole compound (N-phenylcarbazole, polyvinylcarbazole, etc.), a fluorene compound, etc. The hole transfer region 120 may include one of the hole transfer materials described above, or a combination thereof.

The hole transfer materials described above may be included in at least one of the hole injection layer 122, the hole transport layer 124, and the electron blocking layer 126.

The hole transfer region 120 may further include a charge generating material. The charge generating material may be a dopant material such as a p-dopant, so that conductivity of the hole transfer region 120 may be improved.

Examples of the dopant material may include a halogenated metal compound such as LiF, NaCl, CsF, RbCl, RbI, CuI, and KI; a quinone derivative such as TCNQ (tetracyanoquinodimethane), F4-TCNQ (2,3,5,6-tetrafluoro-7,7,8,8-tetracyanoquinodimethane), etc.; a cyano-containing compound such as HATCN (dipyrazino[2,3-f: 2′,3′-h quinoxaline-2,3,6,7,10,11-hexacarbonitrile), NDP9 (4-[[2,3-bis[cyano-(4-cyano-2,3,5,6-tetrafluorophenyl)methylidenecyclopropylidene-cyanomethyl-2,3,5,6-tetrafluorobenzonitrile), etc.; a tungsten (W) oxide; a molybdenum (Mo) oxide; etc. The hole transfer region 120 may include one of the dopant materials described above, or a combination thereof.

A thickness of the hole transfer region 120 may be in a range of about 100 Å to about 10,000 Å. For example, the thickness of the hole transfer region 120 may be in a range of about 100 Å to about 1,500 Å.

When the hole transfer region 120 includes the hole injection layer 122 or the hole transport layer 124, a thickness of the hole injection layer 122 may be in a range from about 100 Å to about 9,000 Å, from about 100 Å to about 3,000 Å, or from about 100 Å to about 1,000 Å. A thickness of the hole transport layer 124 may be in a range from 50 Å to about 2,000 Å, from about 100 Å to about 1,500 Å, from about 100 Å to about 1,000 Å, or from about 100 Å to about 600 Å.

In the thickness ranges described above, hole transfer properties may be enhanced even at a low voltage operation, and a life-span of the device may be further improved.

Each layer of the hole transfer region 120 may be formed by a process such as a thermal evaporation deposition, a vacuum deposition, a spin coating, an inkjet printing, a laser printing, a casting, a laser thermal transfer, etc.

The electron transfer region 140 may be disposed between the second electrode 150 and the emission layer 130. The electron transfer region 140 may have a single-layered, or a multi-layered structure including different materials.

The electron transfer region 140 may include an electron injection layer, an electron transport layer, and/or a hole blocking layer, and may further include an auxiliary emission layer.

In embodiments, as illustrated in FIG. 2, the electron transfer region 140 may include an electron injection layer 142 and an electron transport layer 144, stacked from the second electrode 150 to the emission layer 130.

In some embodiments, as illustrated in FIG. 3, the electron transfer region 140 may include an electron injection layer 142, an electron transport layer 144, and a hole blocking layer 146, sequentially stacked from the second electrode 150. The hole blocking layer 146 may block or suppress a hole transfer from the hole transfer region 120. Accordingly, emission energy and luminescence efficiency in the emission layer 130 may be further improved.

For example, the electron transfer region 140 may include a compound represented by Chemical Formula ET.

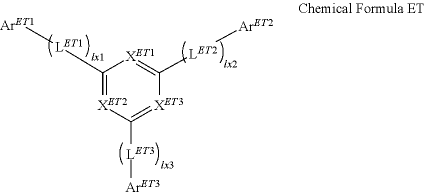

In Chemical Formula ET, at least one of X^{ET1} to X^{ET3} may be N; and the remainder of X^{ET1} to X^{ET3} may each independently be C(R^{ET}). R^{ET} may be a hydrogen atom, a deuterium atom, a substituted or unsubstituted C₁-C₂₀ alkyl group, a substituted or unsubstituted C₆-C₆₀ aryl group, a substituted or unsubstituted C₆-C₆₀ alkyl aryl group, a substituted or unsubstituted C₆-C₆₀ aryl alkyl group, a substituted or unsubstituted C₂-C₆₀ heteroaryl group, a substituted or unsubstituted C₂-C₆₀ alkyl heteroaryl group, or a substituted or unsubstituted C₂-C₆₀ heteroaryl alkyl group.

When one of X^{ET1} to X^{ET3} is N, the compound represented by Chemical Formula ET may include a pyridine group. When two of X^{ET1} to X^{ET3} are N, the compound represented by Chemical Formula ET may include a pyrimidine group. When X^{ET1} to X^{ET3} are each N, the compound represented by Chemical Formula ET may include a triazine group.

In Chemical Formula ET, lx1 to lx3 may each independently be an integer from 0 to 10. L^{ET1} to L^{ET3} may each independently be a direct linkage, a substituted or unsubstituted C₆-C₃₀ arylene group, or a substituted or unsubstituted C₂-C₃₀ heteroarylene group.

When lx1, lx2, or lx3 is 2 or more, two or more of each of L^{ET1}, L^{ET2}, or L^{ET3}, respectively, may be directly linked together, e.g., by carbon atoms of each aryl ring (e.g., sp2 carbons), to form a substituted or unsubstituted C₆-C₃₀ arylene group, or a substituted or unsubstituted C₂-C₃₀ heteroarylene group.

In Chemical Formula ET, Ar^{ET1} to Ar^{ET3} may each independently be a hydrogen atom, a deuterium atom, a substituted or unsubstituted C₁-C₂₀ alkyl group, a substituted or unsubstituted C₆-C₃₀ aryl group, a substituted or unsubstituted C₆-C₆₀ alkyl aryl group, a substituted or unsubstituted C₆-C₆₀ aryl alkyl group, a substituted or unsubstituted C₂-C₃₀ heteroaryl group, a substituted or unsubstituted C₂-C₃₀ alkyl heteroaryl group, or a substituted or unsubstituted C₂-C₃₀ heteroaryl alkyl group. For example, Ar^{ET1} to Ar^{ET3} may each independently be a substituted or unsubstituted phenyl group, a substituted or unsubstituted carbazole group, a substituted or unsubstituted fluorene group, or a substituted or unsubstituted silyl group. The silyl group may be represented by —Si(R^{sa}) (R^{sb}) (R^{sc}), as explained above.

For example, the electron transfer region 140 may include an anthracene compound, Alq₃ (tris(8-hydroxyquinolinato)aluminum), 1,3,5-tri[(3-pyridyl)-phen-3-yl]benzene, 2,4,6-tris(3′-(pyridin-3-yl) biphenyl-3-yl)-1,3,5-triazine, 2-(4-(N-phenylbenzimidazol-1-yl)phenyl)-9,10-dinaphthylanthracene, TPBi (1,3,5-Tri (1-phenyl-1H-benzo[d]imidazol-2-yl)benzene), BCP (2,9-dimethyl-4,7-diphenyl-1,10-phenanthroline), Bphen (4,7-diphenyl-1,10-phenanthroline), TAZ (3-(4-biphenylyl)-4-phenyl-5-tert-butylphenyl-1,2,4-triazole), NTAZ (4-(naphthalen-1-yl)-3,5-diphenyl-4H-1,2,4-triazole), tBu-PBD (2-(4-biphenylyl)-5-(4-tert-butylphenyl)-1,3,4-oxadiazole), BAlq (Bis(2-methyl-8-quinolinolato-N1,O8)-(1,1′-biphenyl-4-olato)aluminum), Bebq₂ (beryllium bis(benzoquinolin-10-olate)), ADN (9,10-di(naphthalene-2-yl) anthracene), BmPyPhB (1,3-bis[3,5-di(pyridin-3-yl)phenylbenzene), or the like. The electron transfer region 140 may include one of the electron transfer materials described above, or a combination thereof.

The above-mentioned material may be included in at least one of the electron injection layer 142, the electron transport layer 144, and the hole blocking layer 146.

The electron transfer region 140 may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or a combination thereof. In some embodiments, the above-mentioned material may be included electron injection layer 142.

The alkali metal may include Li, Na, K, Rb, Cs, or any combination thereof. The alkaline earth metal may include Mg, Ca, Sr, Ba, or any combination thereof. The rare earth metal may include Sc, Y, Ce, Tb, Yb, Gd, or any combination thereof.

The alkali metal-containing compound, the alkaline earth metal-containing compound, and the rare earth metal-containing compound may include an oxide, a halide (e.g., a fluoride, a chloride, a bromide, an iodide, etc.), a telluride, or a combination thereof of the alkali metal, the alkaline earth metal, and the rare earth metal, respectively.

The alkali metal complex, the alkaline earth metal complex, and the rare earth metal complex may include a metal ion such as an alkali metal ion, an alkaline earth metal ion or a rare earth metal ion, and a ligand bonded to the metal ion. The ligand may include, e.g., hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenylbenzimidazole, hydroxyphenylbenzothiazole, bipyridine, phenanthroline, cyclopentadiene, or a combination thereof.

A thickness of the electron transfer region 140 may be in a range from about 100 Å to about 1,000 Å, e.g., from about 150 Å to about 500 Å.

When the electron transfer region 140 includes an electron injection layer 142 or an electron transport layer 144, a thickness of the electron injection layer 142 may be in a range from about 1 Å to about 100 Å, from 1 Å to about 90 Å or from about 5 Å to about 50 Å, and a thickness of the electron transport layer 144 may be in a range from 10 Å to about 900 Å, from about 10 Å to about 500 Å or from about 100 Å to about 400 Å.

Within any of the thickness ranges described above, electron injection and electron transport properties may be further improved without an excessive increase in driving voltage, and stability of the electron transfer region 140 may be improved.

Each layer of the electron transfer region 140 may be formed by a process such as a thermal evaporation deposition, a vacuum deposition, a spin coating, an inkjet printing, a laser printing, a casting, a laser thermal transfer, etc.

The light-emitting device ED may further include a capping layer. Light emission efficiency to an outside of the light-emitting device ED may be improved through the capping layer.

As illustrated in FIG. 4, a second capping layer 160b may be formed on an outer surface of the second electrode 150. In some embodiments, a first capping layer 160a may be formed on an outer surface of the first electrode 110.

A refractive index of the first capping layer 160a and/or the second capping layer 160b may be 1.6 or more. For example, the refractive index of the first capping layer 160a and/or the second capping layer 160b may be 1.6 or more, 1.8 or more, or 2.0 or more for a light in a wavelength range of 550 nm to 660 nm.

The first capping layer 160a and the second capping layer 160b may each be formed as an organic capping layer including an organic material, an inorganic capping layer including an inorganic material, or an organic-inorganic hybrid capping layer including both the organic and inorganic materials.

The first capping layer 160a and/or the second capping layer 160b may each have a single-layered structure or a multi-layered structure including different materials.

In some embodiments, the first capping layer 160a and the second capping layer 160b may each independently include a carbocyclic compound, a heterocyclic compound, an amine group-containing compound, a porphine derivative, a phthalocyanine derivative, a naphthalocyanine derivative, an alkaline metal complex, an alkaline earth metal complex, etc. The first capping layer 160a and the second capping layer 160b may each independently include one of the aforementioned materials, or a combination thereof.

In some embodiments, the first capping layer 160a and/or the second capping layer 160b may each independently include an amine group-containing compound.

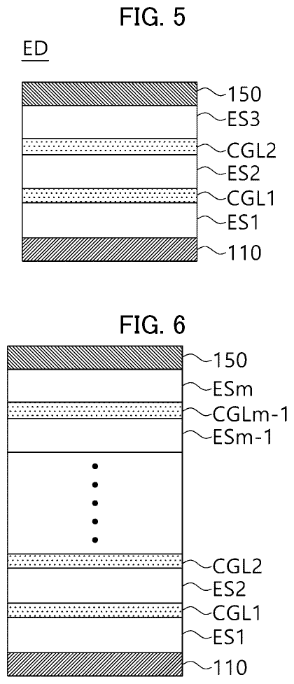

Referring to FIG. 5, the light-emitting device ED may include a plurality of light-emitting structures (e.g., the light-emitting structures ES1, ES2 and ES3). The light-emitting structures ES1, ES2, and ES3 may each include a stacked structure of the hole transfer region 120, the emission layer 130, and the electron transfer region 140, as described with reference to FIGS. 1 to 4. In example embodiments, the light-emitting device ED of FIG. 5 may be a light-emitting device having a tandem structure.

Charge generation layers CGL1 and CGL2 may each be disposed between adjacent structures among the light-emitting structures ES1, ES2 and ES3. Charge generation layers CGL1 and CGL2 may each independently include a p-type charge generation layer and/or an n-type charge generation layer.

The p-type charge generation layer may include a hole transport host compound, such as NPB. For example, the p-type charge generation layer may include a compound represented by Chemical Formula HT as described above. The p-type charge generation layer may further include a p-dopant, such as TCNQ.

The n-type charge generation layer may include an electron transport host compound. For example, the n-type charge generation layer may include a compound represented by Chemical Formula ET as described above. In some embodiments, the n-type charge generation layer may include a phenanthroline-based compound.

The charge generation layers CGL1 and CGL2 may include a first charge generation layer CGL1 disposed between the first light-emitting structure ES1 and the second light-emitting structure ES2, and a second charge generation layer CGL2 disposed between the second light-emitting structure ES2 and the third-light emitting structure ES3.

In example embodiments, the first light-emitting structure ES1, the first charge generation layer CGL1, the second light-emitting structure ES2, the second charge generation layer CGL2, the third light-emitting structure ES3, and the second electrode 150 may be sequentially stacked from a top surface of the first electrode 110.

Colors emitted from the first light-emitting structure ES1, the second light-emitting structure ES2 and the third light-emitting structure ES3 may be the same or different from each other. In some embodiments, the first light-emitting structure ES1, the second light-emitting structure ES2 and the third light-emitting structure ES3 may include a red light-emitting layer, a green light-emitting layer, and a blue light-emitting layer, respectively, and a white light-emitting structure may be implemented through the tandem structure, but is not limited thereto.

In FIG. 5, the 3-stack tandem structure in which three light-emitting structures are stacked is illustrated as an example, but the tandem structure of the light-emitting device of the present disclosure is not limited to the structure illustrated in FIG. 5. For example, 2-stack structure, or a 4-stack structure, a 5-stack structure, or more stacked structure as will be described with reference FIG. 6 may also be implemented.

Referring to FIG. 6, as described with reference to FIG. 5, a tandem structure in which the light-emitting structure and a charge generation layer are alternately and repeatedly stacked may be disposed between the first electrode 110 and the second electrode 150.

In example embodiments, first to m^{th} light-emitting structures ES1 to ESm may be sequentially stacked from the top surface of the first electrode 110 with the charge generation layer interposed therebetween. Each light-emitting structure may be measured from 1 to m^{th} starting with ES1. The charge generation layer may include a first charge generation layer CGL1 to an (m−1)^{th} charge generation layer CGLm-1 sequentially stacked from the top surface of the first electrode 110.

As illustrated in FIG. 6, the first light-emitting structure ES1, the first charge generation layer CGL1, the second light-emitting structure ES2, the second charge generation layer CGL2, . . . , an (m−1)^{th} light-emitting structure ESm-1, an (m−1)^{th} charge generation layer CGLm-1, an m^{th} light-emitting structure ESm, and the second electrode 150 may be sequentially stacked from the top surface of the first electrode 110.

In some embodiments, m is 4, and an intermediate layer of the light-emitting device may have a 4-stack tandem structure, and may include first to fourth light-emitting structures ES1, ES2, ES3 and ES4, and first to third charge generation layers CGL1, CGL2 and CGL3. Colors of light generated from the first to fourth light-emitting structures ES1, ES2, ES3 and ES4 may be the same or different from each other.

In some embodiments, the first to fourth light emitting structures ES1, ES2, ES3 and ES4 may include at least one blue light-emitting structure and at least one green-light emitting structure. In a non-limiting example, the first to third light emitting structures ES1, ES2 and ES3 may correspond to the blue light-emitting structure, and the fourth light emitting structure ES4 may correspond to the green-light emitting structure.

In some embodiments, m is 5, and an intermediate layer of the light-emitting device may have a 5-stack tandem structure, and may include first to fifth light-emitting structures ES1, ES2, ES3, ES4 and ES5, and first to fourth charge generation layers CGL1, CGL2, CGL3 and CGL4. Colors of light generated from the first to fifth light-emitting structures ES1, ES2, ES3, ES4, and ES5 may be the same or different from each other.

In some embodiments, the first to fifth light-emitting structures ES1, ES2, ES3, ES4 and ES5 may include at least one blue light emitting structure and at least one green light emitting structure. In a non-limiting example, the first to fifth light-emitting structures ES1, ES2, ES3, ES4 and ES5 may include three blue light-emitting structures and two green light-emitting structures. For example, the first, third and fifth light-emitting structures ES1, ES3 and ES5 may correspond to the blue light-emitting structure, and the second and fourth light-emitting structures ES2 and ES4 may correspond to the green light-emitting structure.

### Electronic Device

The above-described light-emitting device ED may be applied to an electronic device and may be provided as a light-emitting portion or a light-emitting unit of the electronic device.

Examples of an electronic device may include a display device, a billboard, a signboard, a light source, a lighting device, a personal computer such as a laptop computer or a desktop computer, a mobile phone, an electronic book, an electronic dictionary, an electronic notebook, a health-care device including a diagnostic device and various sensors, various display parts for transportation means (automobile, aircraft, ship, train, etc.).

In example embodiments, the light-emitting device ED may be applied to an organic light emitting diode (OLED) display device or a quantum dot (QD)-OLED display device.

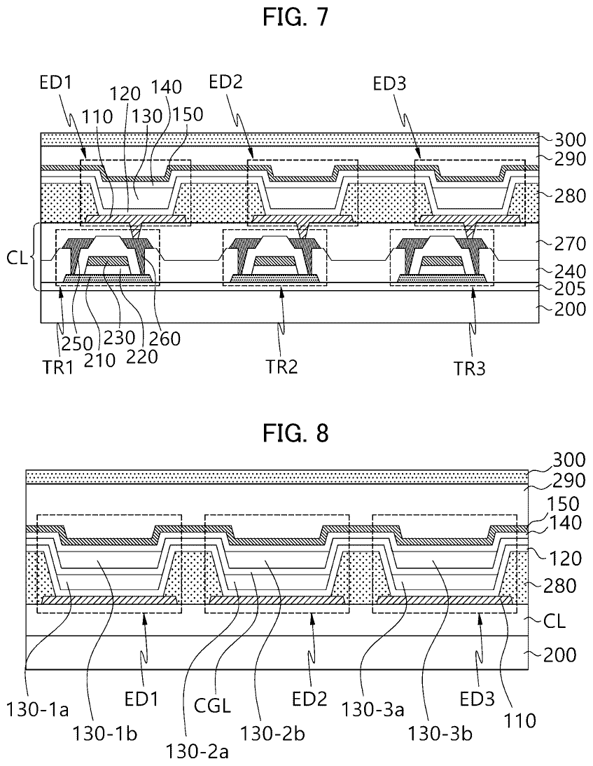

FIG. 7 is a schematic cross-sectional view illustrating a display device in accordance with example embodiments.

Referring to FIG. 7, the display device may include a circuit layer CL disposed on a base substrate 200, and light-emitting devices ED1, ED2 and ED3 disposed on the circuit layer CL.

The base substrate 200 may serve as a supporting substrate or as a back-plane substrate of a display device. The base substrate 200 may be a glass substrate or a plastic substrate.

In some embodiments, the base substrate 200 may include a polymer material having transparent and flexible properties. When the base substrate 200 includes a polymer material, the base substrate 200 may be used in a transparent flexible display device. For example, the base substrate 200 may include a polymer material such as polyimide, polysiloxane, an epoxy resin, an acrylic resin, polyester, etc. In some embodiments, the base substrate 200 may include polyimide.

The circuit layer CL may include transistors TR1, TR2 and TR3. The circuit layer CL may include wiring layers and insulating layers that form a thin film transistor array (TFT-Array).

The circuit layer CL may further include a buffer layer 205 on a top surface of the base substrate 200. The buffer layer 205 may block the penetration of moisture through the base substrate 200, and may also block the diffusion of impurities between the base substrate 200 and the structures formed thereon.

The buffer layer 205 may include, e.g., silicon oxide, silicon nitride, or silicon oxynitride. The buffer layer 205 may include one of the aforementioned materials, or a combination thereof. In some embodiments, the buffer layer 205 may have a stacked structure that includes a silicon oxide layer and a silicon nitride layer.

The transistors TR1, TR2 and TR3 may be disposed on the buffer layer 205. A first transistor TR1, a second transistor TR2 and a third transistor TR3 may be electrically connected to a first light-emitting device ED1, a second light-emitting device ED2 and a third light-emitting device ED3, respectively.

The transistors TR1, TR2 and TR3 may each include an active layer 210, a gate insulation layer 220, and a gate electrode 230.

The active layer 210 may be disposed on the buffer layer 205, and may be patterned for each pixel. The active layer 210 may include a silicon compound such as amorphous silicon or polysilicon. A p-type dopant or an n-type dopant may be doped in a region of the active layer 210, and the active layer 210 may include a source region, a drain region, and a channel region.

The active layer 210 may include an oxide semiconductor, such as indium gallium zinc oxide (IGZO), zinc tin oxide (ZTO), or indium tin zinc oxide (ITZO).

The gate insulation layer 220 may be formed on the active layer 210, and the gate electrode 230 may be stacked on the gate insulation layer 220. As illustrated in FIG. 7, the gate insulation layer 220 may be patterned to partially cover each active layer 210. Alternatively, the gate insulation layer 220 may extend continuously over multiple pixels or light-emitting regions, and may be provided as a common layer for the first, second, and third transistors TR1, TR2 and TR3.

The gate electrode 230 may overlap the channel region of the active layer 210 in a thickness direction.

An insulating interlayer 240 may be formed on the active layer 210 to cover the gate electrode 230 and the gate insulation layer 220. Connection electrodes 250 and 260 which may be in contact with or electrically connected to the active layer 210 may each be disposed on the insulating interlayer 240.

The connection electrodes 250 and 260 may extend through the insulating interlayer 240 to be in contact with or electrically connected to the active layer 210. When the gate insulation layer 220 is provided as a common layer for multiple light-emitting regions, the connection electrodes 250 and 260 may also extend through the gate insulation layer 220.

The connection electrodes 250 and 260 may include a source electrode 250 that may be in contact with or connected to the source region of the active layer 210, and a drain electrode 260 that may be in contact with or connected to the drain region of the active layer 210.

The gate insulation layer 220 and the insulating interlayer 240 may each independently include silicon oxide, silicon nitride, or silicon oxynitride, and may each have a stacked structure that includes a silicon oxide layer and a silicon nitride layer.

The gate electrode 230 and the connection electrodes 250 and 260 may include a metal such as Ag, Mg, Al, W, Cu, Ni, Cr, Mo, Ti, Pt, Ta, Nd, Sc, an alloy thereof, or a nitride thereof.

A via insulation layer 270 may be formed on the insulating interlayer 240 to cover the connection electrodes 250 and 260.

The via insulation layer 270 may accommodate a via structure electrically connecting the first electrode 110 and the drain electrode 260. The via insulation layer 270 may serve as a planarization layer of the circuit layer CL. In embodiments, the via insulation layer 270 may include an organic material such as polyimide, an epoxy resin, an acrylic resin, polyester, etc.

The light-emitting devices ED1, ED2 and ED3 may be disposed on the via insulation layer 270. For example, as described with reference to FIGS. 1 to 4, the light-emitting devices ED1, ED2 and ED3 may include the first electrode 110, the hole transfer region 120, the emission layer 130, the electron transfer region 140, and the second electrode 150 which are sequentially stacked from the via insulation layer 270.

The first electrode 110 may be electrically connected to the transistors TR1, TR2 and TR3 or the connection electrodes 250 and 260 in the circuit layer CL through the via structure. As illustrated in FIG. 7, the first electrode 110 may be in contact with or connected to the drain electrode 260 to serve as a pixel electrode patterned for each light-emitting region or pixel.

A pixel defining layer 280 may be formed on the via insulation layer 270 to define each light-emitting region or pixel. For example, a red light-emitting region, a green light-emitting region, and a blue light-emitting region may be separated and defined by the pixel defining layer 280, and the light-emitting devices ED1, ED2, and ED3 may respectively correspond to a red light-emitting device, a green light-emitting device, and a blue light-emitting device.

The pixel defining layer 280 may partially cover the first electrode 110 of each light-emitting region.

As illustrated in FIG. 7, the hole transfer region 120 and the electron transfer region 140 may each be provided as a common layer that continuously extends over the pixel defining layer 280 and the first electrodes 110. The emission layer 130 may be formed within each light emitting-region or pixel, and may be separated by the pixel defining layer 280.

In some embodiments, the emission layer 130 may also be provided as a common layer that continuously extends over the light emitting-regions or pixels. In some embodiments, the hole transfer region 120, the emission layer 130, and the electron transfer region 140 may each be patterned and separately formed for each light-emitting region or pixel.

The second electrode 150 may be provided as a common electrode that continuously extends over the light-emitting regions or the pixels.

An encapsulation layer 290 may be disposed on the pixel defining layer 280 and the light-emitting devices ED1, ED2 and ED3 to protect the light-emitting devices ED1, ED2 and ED3 from moisture and/or oxygen. The encapsulation layer 290 may be a thin film encapsulation (TFE) having a single-layered structure or multi-layered structure.

The encapsulation layer 290 may include an inorganic layer that includes silicon nitride (SiNx), silicon oxide (SiOx), indium tin oxide, indium zinc oxide, or any combination thereof; an organic layer that includes polyethylene terephthalate, polyethylene naphthalate, polycarbonate, polyimide, polyethylene sulfonate, polyoxymethylene, polyarylate, hexamethyldisiloxane, an acrylic resin (e.g., polymethylmethacrylate, polyacrylic acid, etc.), an epoxy resin (e.g., an aliphatic glycidyl ether (AGE)) or any combination thereof; or a combination of the inorganic layer and the organic layer.

The display device may further include a functional layer 300 disposed on the encapsulation layer 290. The functional layer 300 may include a sensor layer such as a touch sensor layer, an optical layer such as a polarizing layer, a color conversion layer, a color filter layer, a window film, or any combination thereof.

FIG. 8 is a schematic cross-sectional view illustrating a display device in accordance with example embodiments.

Referring to FIG. 8, each of the light-emitting devices ED1, ED2 and ED3 may have a tandem structure, e.g., a 2-stack tandem structure.

In some embodiments, the hole transfer region 120 and the electron transfer region 140 may be continuously and commonly formed and included in an intermediate layer of each light-emitting structure. Additionally, a charge generation layer CGL may continuously extend across a plurality of pixels and may be commonly included in the intermediate layer of each light-emitting structure.

The first light-emitting device ED1 may include a first lower emission layer 130-1a disposed between the hole transfer region 120 and the charge generation layer CGL, and a first upper emission layer 130-1b disposed between the charge generation layer CGL and the electron transfer region 140.

The second light-emitting device ED2 may include a second lower emission layer 130-2a disposed between the hole transfer region 120 and the charge generation layer CGL, and a second upper emission layer 130-2b disposed between the charge generation layer CGL and the electron transfer region 140.

The third light-emitting device ED3 may include a third lower emission layer 130-3a disposed between the hole transfer region 120 and the charge generation layer CGL, and a third upper emission layer 130-3b disposed between the charge generation layer CGL and the electron transfer region 140.

The lower and upper emission layers included in each light-emitting structure may generate light of the same color. In some embodiments, each of the first lower emission layer 130-1a and the first upper emission layer 130-1b included in the first light-emitting device ED1 may correspond to a red emission layer. Each of the second lower emission layer 130-2a and the second upper emission layer 130-2b included in the second light-emitting device ED2 may correspond to a green emission layer. Each of the third lower emission layer 130-3a and the third upper emission layer 130-3b included in the third light-emitting device ED3 may correspond to a blue emission layer.

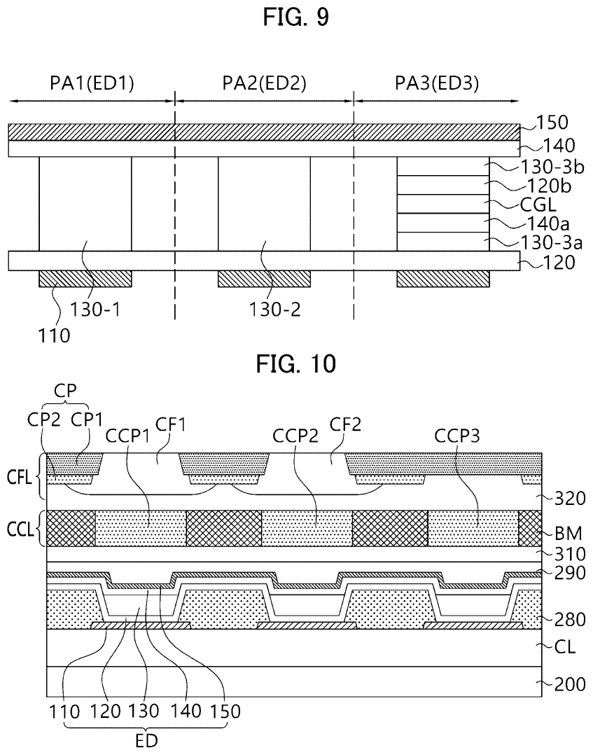

FIG. 9 is a schematic cross-sectional view illustrating a stack construction of light-emitting structure in a display device in accordance with example embodiments. For convenience of illustration and description, illustration of the circuit layer, the base substrate, the pixel defining layer, etc., is omitted from FIG. 9, and a shape of each layer or element in the light-emitting structure is briefly shown as a rectangle.

Referring to FIG. 9, at least one of the light-emitting devices ED1, ED2 and ED3 or pixel areas PA1, PA2 and PA3 may have a tandem structure including a plurality of emission layers, and at least one of the remainder may have a single emission layer structure.

In some embodiments, one of the light-emitting devices ED1, ED2 and ED3 or the pixel areas PA1, PA2 and PA3 may have a tandem structure, and the remainder may have a single emission layer structure.

As illustrated in FIG. 9, the first light-emitting device ED1, the second light-emitting device ED2, and the third light-emitting device ED3 may be included in the first pixel area PA1, the second pixel area PA2, and the third pixel area PA3, respectively. In some embodiments, the first pixel area PA1, the second pixel area PA2, and the third pixel area PA3 may correspond to a red pixel area, a green pixel area, and a blue pixel area, respectively.

The hole transfer region 120, the electron transfer region 140, and the second electrode 150 may each be provided as a common layer continuously extending over the first pixel area PA1, the second pixel area PA2, and the third pixel area PA3.

The first-light emitting device ED1 included in the first pixel area PA1 may include a first emission layer 130-1, and the second light-emitting device ED2 included in the second pixel area PA2 may include a second emission layer 130-2. Each of the first emission layer 130-1 and the second emission layer 130-2 may be a single-layered emission layer.

The third light-emitting device ED3 included in the third pixel area PA3 may have, e.g., a 2-stack tandem structure. The third light-emitting device ED3 may include a third lower emission layer 130-3a and a third upper emission layer 130-3b separated with the charge generation layer CGL interposed therebetween. Each of the third lower emission layer 130-3a and the third upper emission layer 130-3b may correspond to a blue emission layer.

A lower electron transfer region 140a may be disposed between the charge generation layer CGL and the third lower emission layer 130-3a. An upper hole transfer region 120b may be disposed between the charge generation layer CGL and the third upper emission layer 130-3b.

Accordingly, a tandem light-emitting structure in which the first electrode 110, the hole transfer region 120, the third lower emission layer 130-3a, the lower electron transfer region 140a, the charge generation layer CGL, the upper hole transfer region 120b, the third upper emission layer 130-3b, the electron transfer region 140, and the second electrode 150 are sequentially stacked may be disposed in the third pixel area PA3.

FIG. 10 is a schematic cross-sectional view illustrating a display device in accordance with example embodiments.

FIG. 10 illustrates a display device having a QD-OLED structure according to embodiments. Detailed descriptions regarding elements and structures that are the same as or substantially similar to those described with reference to FIG. 7 will not be repeated here.

Referring to FIG. 10, the pixel defining layer 280 and the light-emitting device ED may be disposed on the circuit layer CL, as described above with reference to FIG. 7. In example embodiments, each pixel may emit light of the same wavelength region. In some embodiments, each light-emitting device ED may emit a blue light.

In some embodiments, each light-emitting region may include the light-emitting device having the tandem structure, as described above with respect to FIG. 5. In this case, the intermediate layer of each light-emitting device ED may be provided as a common layer that continuously extends over a plurality of the light-emitting regions.

A color control layer CCL may be disposed on the encapsulation layer 290, and the color control layer CCL may include color control portions CCP1, CCP2, and CCP3.

The color control portions CCP1, CCP2 and CCP3 may each include a light transformer such as a quantum dot or a phosphor. In each of the color control portions CCP1, CCP2 and CCP3, the light transformer may convert a wavelength of a provided light and emit a resulting light.

The color control portions CCP1, CCP2 and CCP3 may be separated or spaced apart from each other by a bank BM. The bank BM may substantially overlap the pixel defining layer 280, and the color control portions CCP1, CCP2 and CCP3 may substantially overlap each of the emission layers 130.

The color control layer CCL may include a first color control portion CCP1 including a first quantum dot that converts a first color light provided from the light-emitting device ED into a second color light, a second color control portion CCP2 including a second quantum dot that converts the first color light into a third color light, and a third color control portion CCP3 that transmits the first color light.

In some embodiments, the first color light, the second color light, and the third color light may be a blue light, a red light, and a green light, respectively. The first quantum dot and the second quantum dot may respectively be a red quantum dot and a green quantum dot.

The color control portions CCP1, CCP2 and CCP3 may each further include a scattering material such as inorganic particles. The third color control portion CCP3 may not include quantum dots and may include the scattering material. The scattering material may include TiO₂, ZnO, Al₂O₃, SiO₂, hollow silica, etc. The scattering material may be one of the aforementioned materials or a combination thereof.

The color control portions CCP1, CCP2, and CCP3 may each further include a binder resin that disperses the quantum dot and the scattering material. The binder resin may include an acrylic resin, a urethane resin, a silicone resin, an epoxy resin, etc.

A color filter layer CFL that includes color filters CF1 and CF2 and a light-shielding portion CP may be disposed on the color control layer CCL.

The color filter layer CFL may include a first filter CF1 that transmits the second color light, a second filter CF2 that transmits the third color light, and a third filter that transmits the first color light. For example, the first filter CF1 may be a red filter, the second filter CF2 may be a green filter, and the third filter may be a blue filter.

The color filters CF1 and CF2 may each include a photosensitive binder resin and a colorant including a pigment and/or a dye. The first filter CF1 may include a red pigment or dye, and the second filter CF2 may include a green pigment or dye.

The light-shielding portion CP may be disposed between the color filters. In some embodiments, the light-shielding portion may include a first light-shielding portion CP1 and a second light-shielding portion CP2 that includes colorants of different colors.

In some embodiments, the first light-shielding portion CP1 may include a blue colorant, and the second light-shielding portion CP2 may include a red colorant or a black colorant. In some embodiments, in the blue light-emitting region, a portion of the first light-shielding portion CP1 may be provided as a blue color filter and may be exposed between the second light-shielding portions CP2, so that an additional color filter (e.g., the third filter) may be omitted.

A first barrier layer 310 may be disposed between the color control layer CCL and the light-emitting device ED (or the encapsulation layer 290). A second barrier layer 320 may be disposed between the color control layer CCL and the color filter layer CFL.

The barrier layers 310 and 320 may each include at least one inorganic layer. For example, the barrier layers 310 and 320 may each independently include silicon nitride, aluminum nitride, zirconium nitride, titanium nitride, hafnium nitride, tantalum nitride, silicon oxide, aluminum oxide, titanium oxide, tin oxide, cerium oxide, silicon oxynitride, etc.

In some embodiments, the barrier layers 310 and 320 may each have a multi-layered structure that further includes an organic layer.

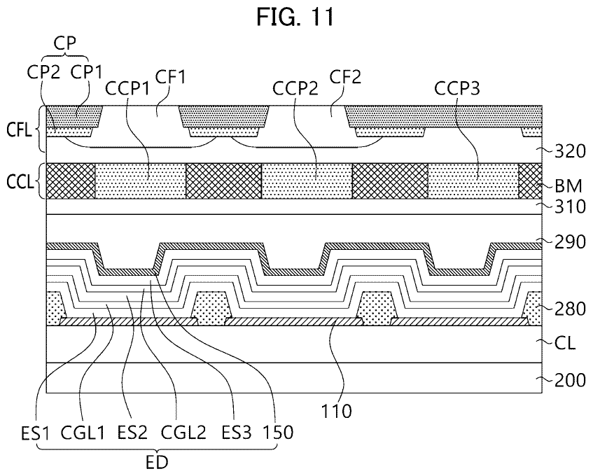

FIG. 11 is a schematic cross-sectional view illustrating a display device in accordance with example embodiments. Detailed descriptions of elements and structures substantially the same as or similar to those described with reference to FIG. 10 are omitted herein.

Referring to FIG. 11, the first light-emitting structure ES1, the first charge generation layer CGL1, the second light-emitting structure ES2, the second charge generation layer CGL2, the third light-emitting structure ES3 and the second electrode 150 of the light-emitting device ED corresponding to the color control portions CCP1, CCP2 and CCP3 may be disposed on the first electrode 110 serving as the pixel electrode, and the light-emitting device ED may have a tandem structure.

In some embodiments, as described with reference to FIG. 5, the first light-emitting structure ES1, the first charge generation layer CGL1, the second light-emitting structure ES2, the second charge generation layer CGL2, and the third light-emitting structure ES3 may be sequentially stacked between the first electrode 110 and the second electrode 150. The first light-emitting structure ES1, the first charge generation layer CGL1, the second light-emitting structure ES2, the second charge generation layer CGL2, and the third light-emitting structure ES3 may be continuously and commonly formed in a plurality of pixel areas or light-emitting regions.

In some embodiments, the first light-emitting structure ES1, the second light-emitting structure ES2, and the third light-emitting structure ES3 may generate different color lights, and the light-emitting device ED may generate a white light. In some embodiments, the first light-emitting structure ES1, the second light-emitting structure ES2, and the third light-emitting structure ES3 may all generate blue lights.

In some embodiments, as described with reference to FIG. 6, the light-emitting device ED may include a tandem structure of 4-stack, 5-stack, or more of the stacked number.

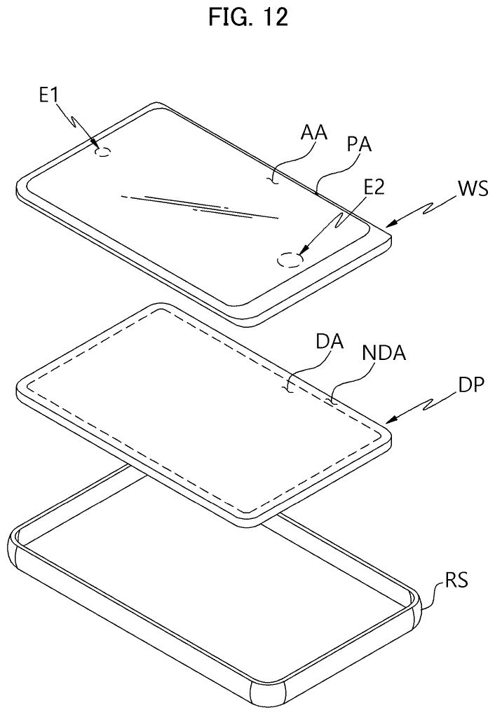

FIG. 12 is a schematic exploded perspective view illustrating an electronic device in accordance with example embodiments.

According to example embodiments, the electronic device may be implemented in the form of a mobile phone (smart phone), a tablet, a PC, or the like, including the above-described display device.

Referring to FIG. 12, the electronic device may include a window structure WS, a display panel DP, and a rear structure RS.

The window structure WS may provide an external display surface recognized by a user, such as a viewing surface of a mobile phone, and may include a transparent material film. For example, the window structure WS may include glass (e.g., ultra-thin glass (UTG), a hard coating film, a plastic film, or the like.

An outer surface of the window structure WS may include an active area AA and a peripheral area PA. The active area AA may provide a surface from which an image of the display device is substantially displayed and to which a user's touch/command is input. The peripheral area PA may substantially correspond to a bezel area of the display device.

The display panel DP may include the above-described display device and may have a display area DA and a non-display area NDA. The display area DA of the display panel DP may substantially correspond to or overlap the active area AA of the window structure WS. The non-display area NDA of the display panel DP may substantially correspond to or overlap the peripheral area PA of the window structure WS.

In some embodiments, functional device areas E1 and E2 may be included in the active area AA of the window structure WS. For example, a first functional device area E1 may be included at one end portion of the active area AA and may be implemented, e.g., in the form of a camera hole. The second functional device area E2 may serve as a fingerprint sensing area.

For example, a sensor structure for a touch sensing or a fingerprint sensing may be disposed in the display panel DP or between the window structure WS and the display panel DP.

The rear structure RS may serve as a frame structure or a housing of the display device or the electronic device. A cover panel may be disposed between the rear structure RS and the display panel DP.

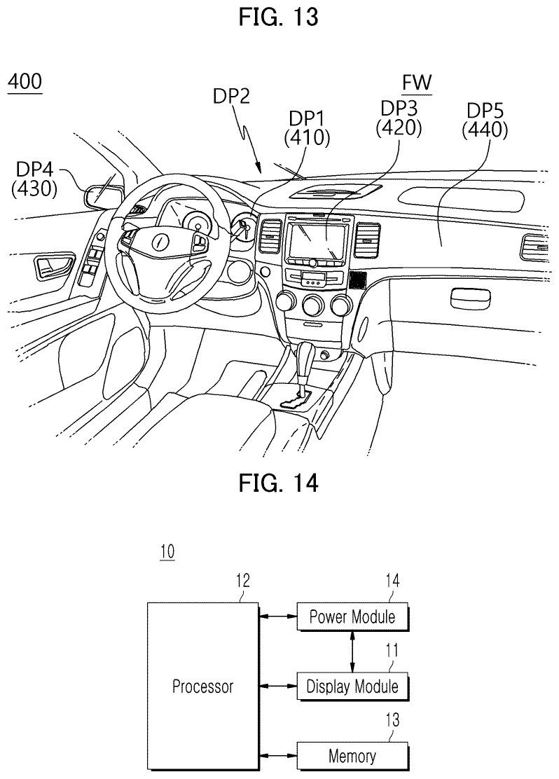

FIG. 13 is a schematic cross-sectional view illustrating an electronic device in accordance with an example embodiment.

The electronic device may be installed in, embedded in, attached to, or integrated with a vehicle 400. However, the vehicle 400 is not limited to the embodiment illustrated in FIG. 13 Further examples of the vehicle 400 may include a transportation means such as a three-wheeled or four-wheeled vehicle, a construction machine, a two-wheeled vehicle, a motor vehicle, a bicycle, a train, etc. Other examples of the vehicle 400 may include an electric vehicle, a hybrid vehicle, etc.

Referring to FIG. 13, at least one of first to fifth display devices DP1, DP2, DP3, DP4, and DP5 may be applied to the vehicle 400.

In example embodiments, the first display device DP1 may be disposed in a cluster area 410. Driving information such as a driving distance and speed, and various warning lights may be displayed in the cluster area 410.

The second display device DP2 may be disposed on a front window FW of the vehicle 400. For example, the second display device DP2 may be installed as a head-up display (HUD).

The third display device DP3 may be disposed on a center fascia 420 of the vehicle 400. In the center fascia 420, a button or a switch for controlling an image display or a music player, an air conditioner, a heater, etc., may be displayed, and vehicle information may be displayed thereon.

The fourth display device DP4 may be applied to side mirrors 430 of the vehicle 400. A side mirror 430 may be installed at each of both sides of an exterior of the vehicle 400, and the fourth display device DP4 may be applied to at least one of the side mirrors 430 installed at each of the both sides.

The fifth display device DP5 may be disposed on a passenger seat dashboard 440. Information/image identical to or different from information/image displayed on the cluster area 410 and/or the center fascia 420 may be displayed at the passenger seat dashboard 440.

The electronic device may include, e.g., a flat panel display, a curved display, a computer monitor, a medical monitor, a television, a billboard, a light for indoor or outdoor lighting, a signal light, a head-up display, a full or partial transparent display, a flexible display, a rollable display, a foldable display, a stretchable display, a laser printer, a phone, a mobile phone, a tablet, a phablet, a personal digital assistant (PDA), a wearable device, a laptop computer, a digital camera, a camcorder, a viewfinder, a micro display, a 3D display, a virtual reality or augmented reality display, a vehicle, a video wall including multiple displays tiled together, a theater or stadium screen, a phototherapy device, or a signage.

The display device according to the embodiments of the present disclosure may be applied to various electronic devices. The electronic device according to some embodiments includes the above-described display device, and may further include a module or device having another additional function in addition to the display device.

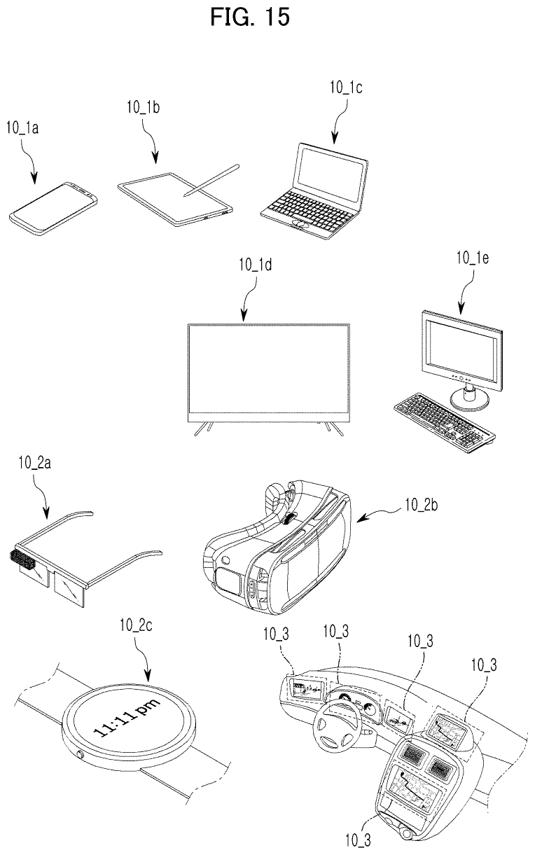

FIG. 14 is a block diagram of an electronic device in accordance with some embodiments.

Referring to FIG. 14, an electronic device 10 according to an embodiment may include a display module 11, a processor 12, a memory 13 and a power module 14.

The display module 11 may include the above-described display device.

The processor 12 may include a central processing unit (CPU), an application processor (AP), a graphic processing unit (GPU), a communication processor (CP), an image signal processor (ISP) and/or a controller.

Data information for an operation of the processor 12 or the display module 11 may be stored in the memory 13. When the processor 12 executes an application stored in the memory 13, an image data signal and/or an input control signal may be transmitted to the display module 11, and the display module 11 may process the received signal and output image information through a display screen.

The power module 14 may include a power supply module such as a power adapter or a battery device, and a power conversion module that converts a power supplied by the power supply module to a generate power required for the operation of the electronic device 10.

At least one of components of the electronic device 10 as described above may be included in the display device according to the above-described embodiments. Additionally, some of individual modules functionally included in one module may be included in the display device, and others may be provided separately from the display device. For example, the display module 11 may include the display device, and the processor 12, the memory 13 and the power module 14 may be provided in the form of another device in the electronic device 10 different from the display device.

FIG. 15 is a schematic diagram of an electronic device in accordance with various embodiments.

Referring to FIG. 15, non-limiting examples of various electronic devices to which the display device according to the above-described embodiments is applied include an electronic device for displaying an image such as a smartphone 10_1a, a tablet PC 10_1b, a laptop 10_1c, a TV 10_1d, a desk monitor 10_1e, or the like; a wearable electronic device including a display module such as smart glasses 10_2a, a head mounted display 10_2b, a smart watch 10_2c, or the like; a vehicle electronic device 10_3 including a display module such as a center information display (CID) disposed at a vehicle instrument panel, a center fascia, a dashboard, etc., a room mirror display, a head-up display, or the like. The electronic device may include a virtual reality glass or an augmented reality glass.

The electronic device may include, e.g., a flat panel display, a curved display, a computer monitor, a medical monitor, a television, a billboard, a light for indoor or outdoor lighting, a signal light, a head-up display, a full or partial transparent display, a flexible display, a rollable display, a foldable display, a stretchable display, a laser printer, a phone, a mobile phone, a tablet, a phablet, a personal digital assistant (PDA), a wearable device, a laptop computer, a digital camera, a camcorder, a viewfinder, a micro display, a 3D display, a virtual reality or augmented reality display, a vehicle, a video wall including multiple displays tiled together, a theater or stadium screen, a phototherapy device, or a signage.

Hereinafter, embodiments of the present inventive concepts will be described in detail with reference to Experimental Examples including Examples and Comparative Examples. The Examples are provided to assist in understanding the disclosure, but they are provided as non-limiting examples, and the scope of the disclosure is not limited thereto. It will be clear to those skilled in the art that various changes and modifications to disclosed examples can be made within the scope of the disclosure.

### SYNTHESIS EXAMPLE

### Synthesis Example 1: Synthesis of Compound 1

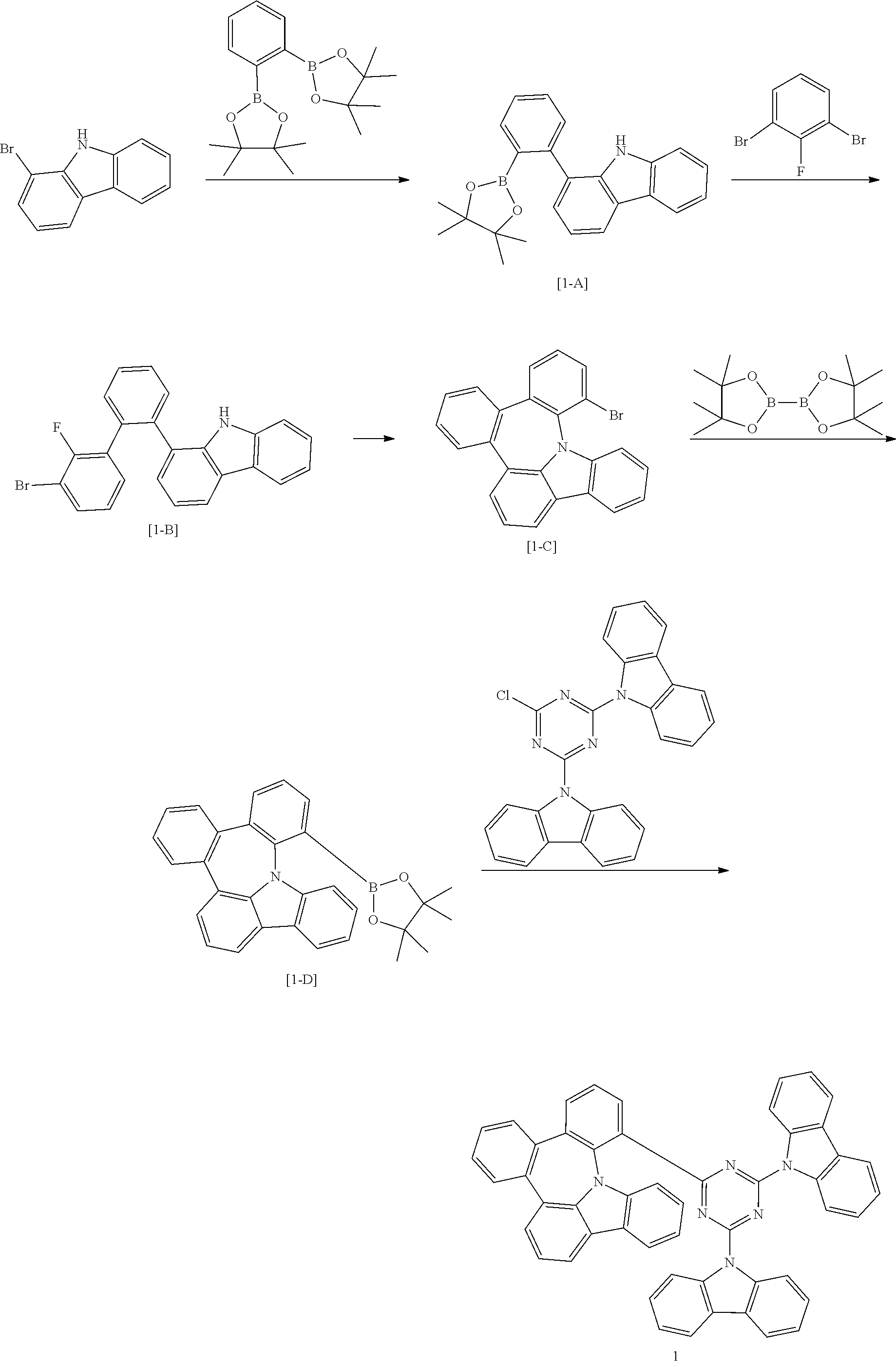

### 1) Synthesis of Intermediate 1-A

4.3 g (17.5 mmol) of 1-bromo-9H-carbazole, 6.9 g (21.0 mmol) of 1,2-bis(4,4,5,5-tetramethyl-1,3,2-dioxaborolan-2-yl)benzene, (0.88 mmol) 1.01 g of tetrakis(triphenylphosphine) palladium (0), and 6.1 g (43.8 mmol) of potassium carbonate were placed in a reaction vessel and suspended in 180 mL of toluene. A reaction temperature was increased to 110° C. and stirred for 12 hours. Thereafter, the mixture was cooled to room temperature and extracted with ethyl acetate. An extracted organic layer was washed with a saturated sodium chloride aqueous solution and dried over sodium sulfate. A residue after removing the solvent was separated using column chromatography to obtain an intermediate 1-A (2.01 g, 5.43 mmol).

### 2) Synthesis of Intermediate 1-B

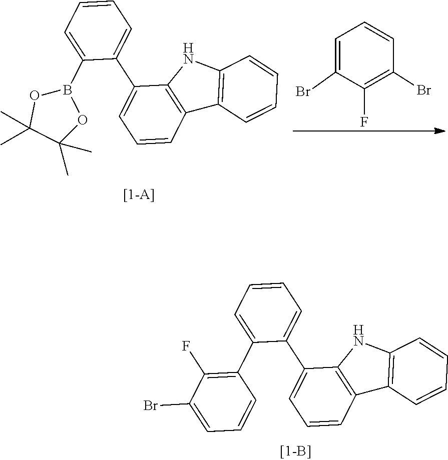

2.01 g (5.43 mmol) of the intermediate 1-A, 1.66 g (6.52 mmol) of 1,3-dibromo-2-fluorobenzene, 310 mg (0.27 mmol) of tetrakis(triphenylphosphine) palladium (0), and potassium carbonate 1.89 g (13.6 mmol) were placed in a reaction vessel and suspended in 50 mL of toluene. A reaction temperature was increased to 110° C. and stirred for 12 hours. Thereafter, the mixture was cooled to room temperature and extracted with ethyl acetate. An extracted organic layer was washed with a saturated sodium chloride aqueous solution and dried over sodium sulfate. A residue from which the solvent was removed was separated using column chromatography to obtain an intermediate 1-B (1.38 g, 3.31 mmol).

### 3) Synthesis of Intermediate 1-C

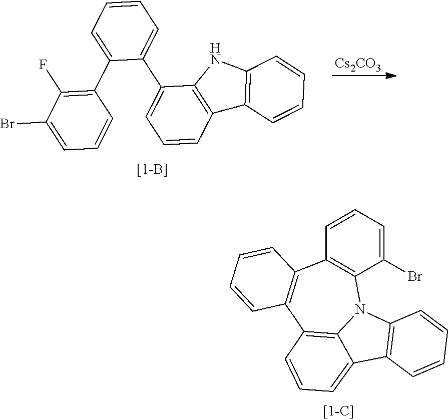

1.38 g (3.31 mmol) of the intermediate 1-B and 5.41 g (16.6 mmol) of cesium carbonate were placed in a reaction vessel and suspended in 30 mL of DMF. The reaction temperature was increased to 160° C. and stirred for 12 hours. Thereafter, the mixture was cooled to room temperature and extracted with ethyl acetate. An extracted organic layer was washed with a saturated aqueous sodium chloride solution and dried over sodium sulfate. A residue after removing the solvent was separated using column chromatography to obtain 1.06 g (2.68 mmol) of the intermediate 1-C.

### 4) Synthesis of Intermediate 1-D

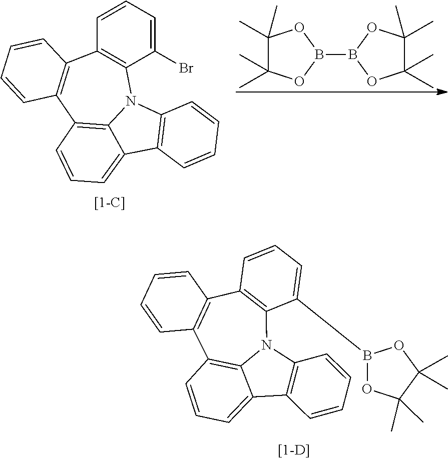

1.06 g (2.68 mmol) of the intermediate 1-C, 1.02 g (4.02 mmol) of 4,4,4′,4′,5,5,5′,5′-octamethyl-2,2′-bi (1,3,2-dioxaborolane), 100 mg (0.13 mmol) of [1,1′-bis(diphenylphosphino) ferrocene]dichloropalladium (II), and 530 mg (5.36 mmol) of potassium acetate were placed in a reaction vessel and suspended in 30 mL of THF. A reaction temperature was increased to 80° C. and stirred for 12 hours. Thereafter, the mixture was cooled to room temperature and extracted with ethyl acetate. An extracted organic layer was washed with a saturated aqueous sodium chloride solution and dried over sodium sulfate. A residue from which the solvent was removed was separated using column chromatography to obtain an intermediate 1-D (860 mg, 1.93 mmol).

### 5) Synthesis of Compound 1

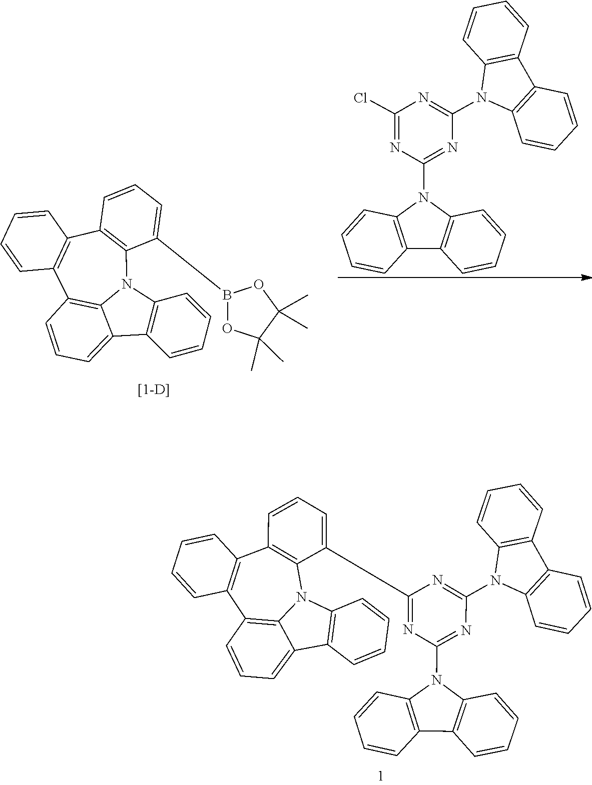 [SMILES: CC1(C)OB(c2cccc3c2-n2c4ccccc4c4cccc(c42)-c2ccccc2-3)OC1(C)C ; Clc1nc(-n2c3ccccc3c3ccccc32)nc(-n2c3ccccc3c3ccccc32)n1 ; c1ccc2c(c1)-c1cccc(-c3nc(-n4c5ccccc5c5ccccc54)nc(-n4c5ccccc5c5ccccc54)n3)c1-n1c3ccccc3c3cccc-2c31]

860 mg (1.93 mmol) of the intermediate 1-D, 950 mg (2.12 mmol) of 9,9′-(6-chloro-1,3,5-triazine-2,4-diyl)bis(9H-carbazole), 110 mg (0.10 mmol) of tetrakis(triphenylphosphine) palladium (0), and 540 mg (3.86 mmol) of potassium carbonate were placed in a reaction vessel and suspended in 20 mL of toluene. A reaction temperature was increased to 110° C. and stirred for 12 hours. Thereafter, the mixture was cooled to room temperature and extracted with ethyl acetate. An extracted organic layer was washed with a saturated sodium chloride aqueous solution and dried over sodium sulfate. A residue after removing the solvent was separated using column chromatography to obtain Compound 1 (990 mg, 1.37 mmol).

### Synthesis Example 2: Synthesis of Compound 10

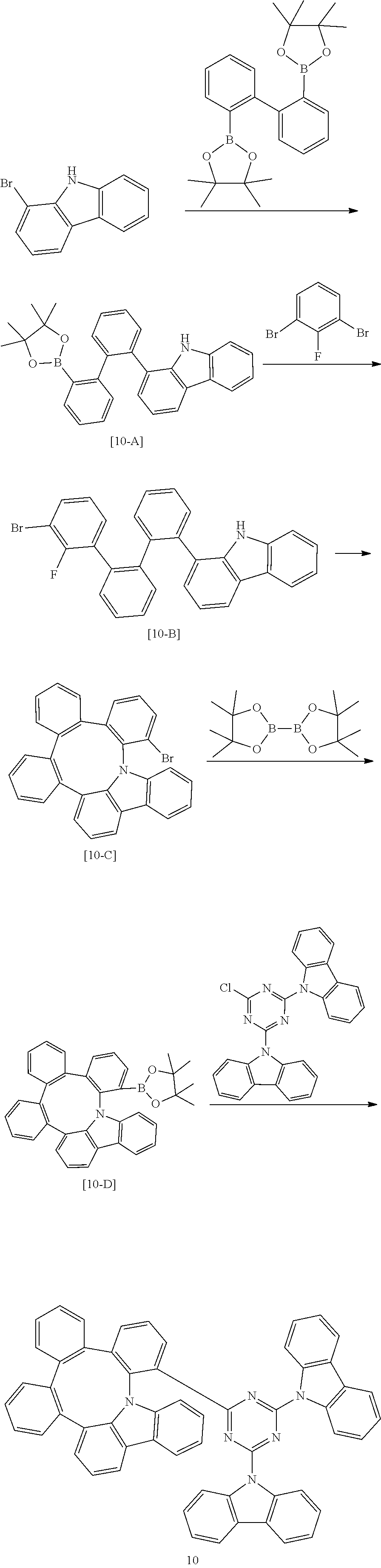

Compound 10 (900 mg, 1.12 mmol) was obtained using the same synthetic scheme as that in Synthesis Example 1, except that 2,2′-bis(4,4,5,5-tetramethyl-1,3,2-dioxaborolan-2-yl)-1,1′-biphenyl was used instead of 1,2-bis(4,4,5,5-tetramethyl-1,3,2-dioxaborolan-2-yl)benzene.

### Synthesis Example 3: Synthesis of Compound 47

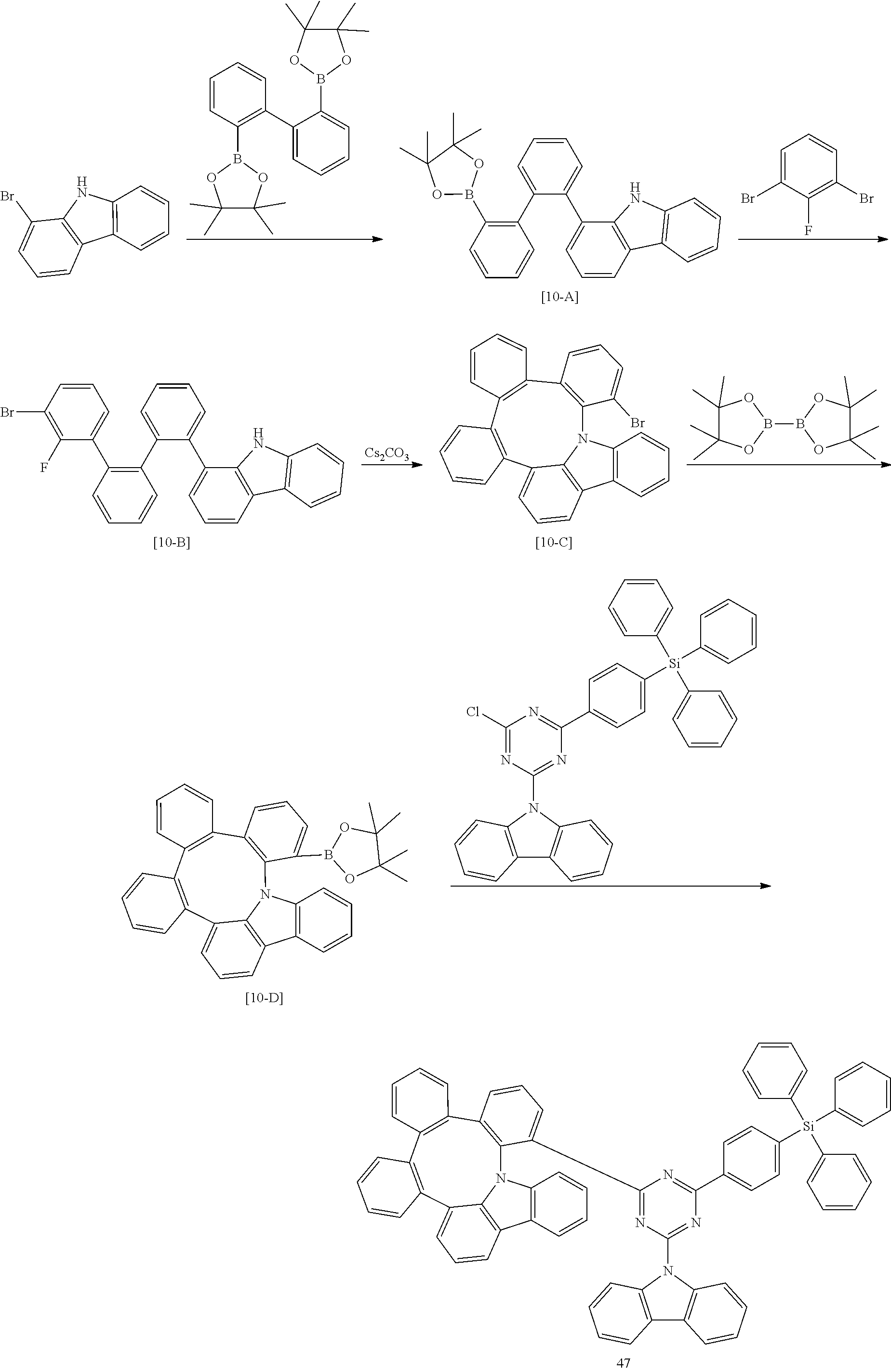

Compound 47 (1.12 g, 1.15 mmol) was obtained using the same synthetic scheme as that in Synthesis Example 2, except that 9-(4-chloro-6-(4-(triphenylsilyl)phenyl)-1,3,5-triazin-2-yl)-9H-carbazole was used instead of 9,9′-(6-chloro-1,3,5-triazine-2,4-diyl)bis(9H-carbazole).

### Synthesis Example 4: Synthesis of Compound 52

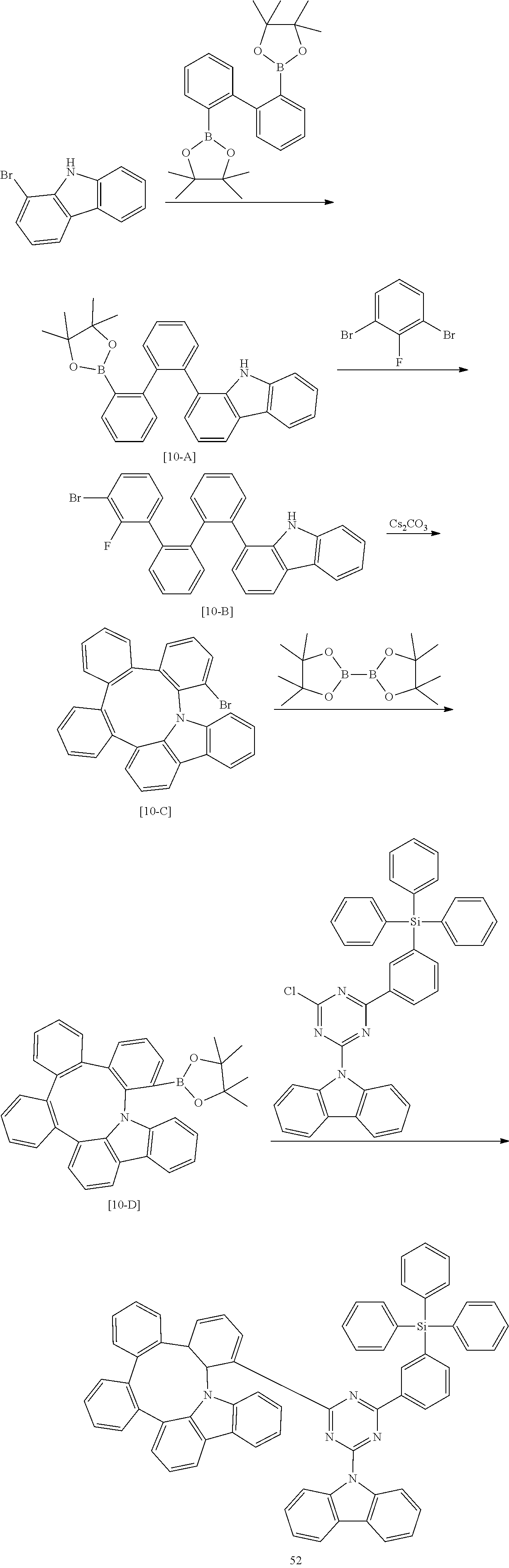

Compound 52 (1.27 g, 1.31 mmol) was obtained using the same synthetic scheme as that in Preparation Example 2, except that 9-(4-chloro-6-(3-(triphenylsilyl)phenyl)-1,3,5-triazin-2-yl)-9H-carbazole was used instead of 9,9′-(6-chloro-1,3,5-triazine-2,4-diyl)bis(9H-carbazole).

TABLE 1

MS/FAB

| Compound No. | found | calc. |
|---|---|---|
| Compound 1 | 726.2537 | 726.2532 |
| Compound 10 | 802.2841 | 802.2845 |
| Compound 47 | 971.3448 | 971.3444 |
| Compound 52 | 971.3451 | 971.3444 |

### Fabrication of Organic Light-Emitting Device 1

As the first electrode, a glass substrate (Corning product) on which a 15 Ω/cm² (1,200 Å) ITO electrode was formed was cut into a size of 50 mm×50 mm×0.5 mm, and the cut substrate was ultrasonically cleaned for 5 minutes using isopropyl alcohol and pure water. The ultrasonically cleaned substrate was irradiated with an ultraviolet ray for 30 minutes and exposed to ozone, and then mounted on a vacuum deposition device.

HATCN was formed as a hole injection layer having a thickness of 100 Å on the substrate, and then BCFN as a first hole transport material was vacuum-deposited on the hole injection layer to form a first hole transport layer having a thickness of 600 Å. Thereafter, BPCbzCbz as a hole transport material was vacuum-deposited as a second hole transport compound on the first hole transport layer to form a second hole transport layer having a thickness of 50 Å.

On the second hole transport layer, BPCbzCbz as a host and light-emission material compounds shown in Table 2 below, and PtON-TBBI as a phosphorescent dopant were simultaneously deposited in a weight ratio of 60:27:13 to form an emission layer having a thickness of 350 Å.

Thereafter, mSiTrz was deposited on the emission layer to form a first electron transport layer having a thickness of 50 Å and then mSiTrz and LiQ were simultaneously deposited in a weight ratio of 1:1 to form a second electron transport layer having a thickness of 350 Å.

LiF as a halogenated alkali metal was deposited on the electron transport layer to form an electron injection layer having a thickness of 15 Å, and Al was vacuum-deposited to a thickness of 80 Å to form a LiF/Al electrode, thereby obtaining an organic light-emitting device 1. The compounds used as mentioned above are represented by chemical formulae below.

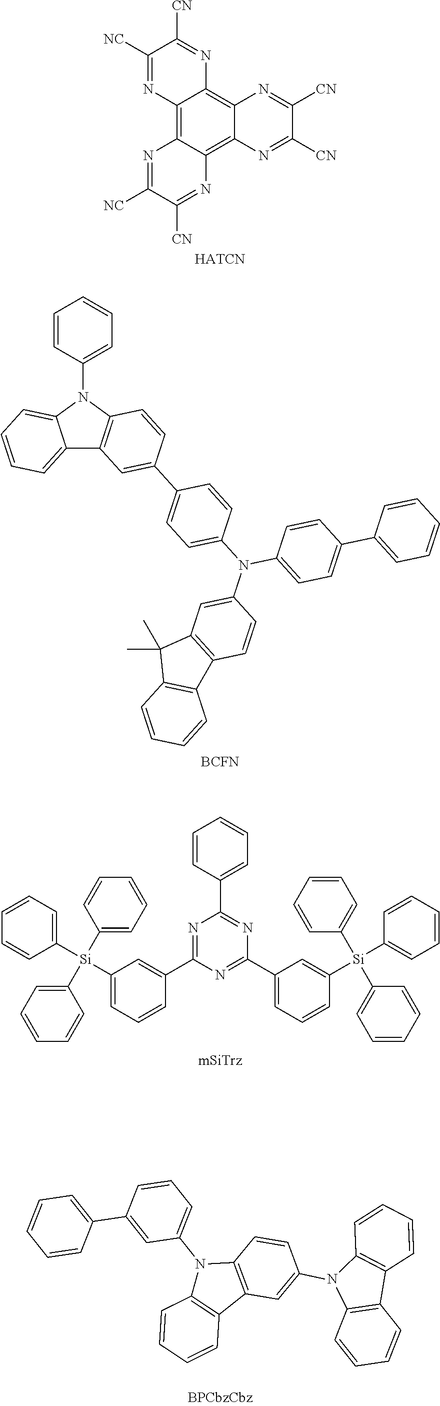

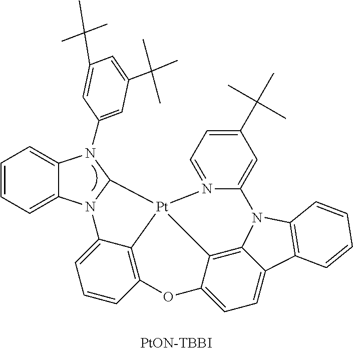

The compounds used in Comparative Examples are as follows.

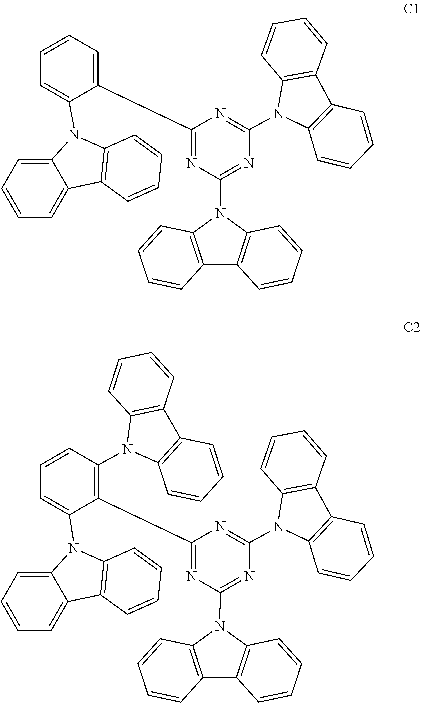 [SMILES: c1ccc(-n2c3ccccc3c3ccccc32)c(-c2nc(-n3c4ccccc4c4ccccc43)nc(-n3c4ccccc4c4ccccc43)n2)c1 ; c1cc(-n2c3ccccc3c3ccccc32)c(-c2nc(-n3c4ccccc4c4ccccc43)nc(-n3c4ccccc4c4ccccc43)n2)c(-n2c3ccccc3c3ccccc32)c1]

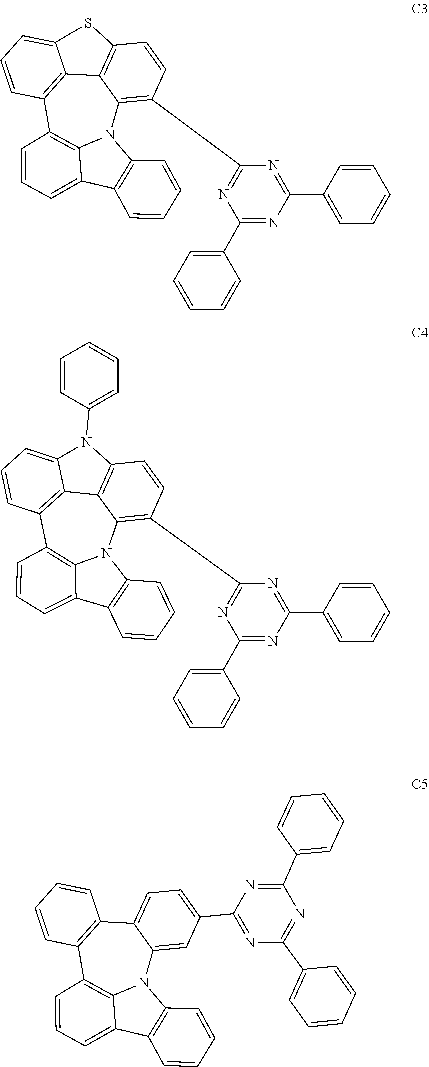 [SMILES: c1ccc(-c2nc(-c3ccccc3)nc(-c3ccc4sc5cccc6c7cccc8c9ccccc9n(c3c4c56)c78)n2)cc1 ; c1ccc(-c2nc(-c3ccccc3)nc(-c3ccc4c5c6c(cccc6n4-c4ccccc4)c4cccc6c7ccccc7n(c35)c46)n2)cc1 ; c1ccc(-c2nc(-c3ccccc3)nc(-c3ccc4c(c3)-n3c5ccccc5c5cccc(c53)-c3ccccc3-4)n2)cc1]

A driving voltage (V), a color conversion efficiency ratio, and a life-span ratio of each organic light-emitting device 1 at 1000 cd/m² were measured using Keithley MU 236 and a luminance meter PR650. The color conversion efficiency ratio and the life-span ratio were measured relatively to a value of Comparative Example 1 set as 100. The evaluation results are shown in Table 2.

TABLE 2

| | Light-emitting | Driving voltage | Color conversion | Life-span | Emission |
|---|---|---|---|---|---|
| | material | (V) | efficiency ratio | ratio | color |
| Example 1 | Compound 1 | 4.5 | 1.15 | 1.14 | blue |
| Example 2 | Compound 10 | 4.6 | 1.21 | 1.21 | blue |
| Example 3 | Compound 47 | 4.4 | 1.22 | 1.30 | blue |
| Example 4 | Compound 52 | 4.3 | 1.24 | 1.39 | blue |
| Comparative | Compound C1 | 4.7 | 1 | 1 | blue |
| Example 1 | | | | | |
| Comparative | Compound C2 | 4.7 | 1.11 | 0.79 | blue |
| Example 2 | | | | | |
| Comparative | Compound C3 | 5.5 | 0.70 | 0.50 | blue |
| Example 3 | | | | | |
| Comparative | Compound C4 | 5.6 | 0.75 | 0.41 | blue |
| Example 4 | | | | | |
| Comparative | Compound C5 | 5.8 | 0.60 | 0.32 | blue |
| Example 5 | | | | | |

Referring to Table 2, when the heterocyclic compound of the present disclosure was used as a host material of the emission layer, the driving voltage of the organic light-emitting device was reduced and the light-emitting efficiency was improved. Additionally, the color conversion efficiency ratio was improved by 1.15 times or more compared to that from Comparative Example 1, and the life-span ratio was also improved by 1.14 times or more compared to that from Comparative Example 1.

The organic light-emitting devices of Comparative Examples did not include the heterocyclic compound of the present disclosure as a host material of the emission layer. Accordingly, Comparative Examples provided higher driving voltage and lower color conversion efficiency ratio and lifespan ratio than those of the light-emitting devices according to Examples.

### Fabrication of Organic Light-Emitting Device 2

An organic light-emitting device 2 was manufactured by the same method as that of the organic light-emitting device 1, except that BPCbzCbz as a host and the light-emitting material compounds shown in Table 3 below, a phosphorescent dopant PtON-TBBI, and tDABNA represented by a chemical formula below were simultaneously deposited in a weight ratio of 60:27:12:1 to form an emission layer having a thickness of 350 Å.

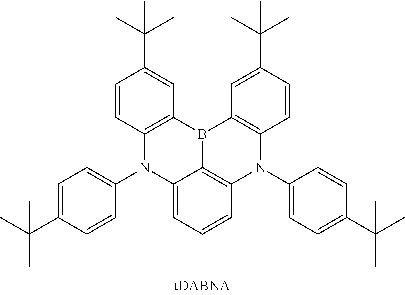 [SMILES: CC(C)(C)c1ccc(N2c3ccc(C(C)(C)C)cc3B3c4cc(C(C)(C)C)ccc4N(c4ccc(C(C)(C)C)cc4)c4cccc2c43)cc1]

A driving voltage (V), a color conversion efficiency ratio, and a life-span ratio of each organic light-emitting device 2 at 1000 cd/m² were measured using Keithley MU 236 and a luminance meter PR650. The color conversion efficiency ratio and the life-span ratio were measured relatively to a value of Comparative Example 1 set as 100. The evaluation results are shown in Table 3.

TABLE 3

| | Light-emitting | Driving voltage | Color conversion | Life-span | Emission |
|---|---|---|---|---|---|
| | material | (V) | efficiency ratio | ratio | color |
| Example 5 | Compound 1 | 4.0 | 1.55 | 1.35 | blue |
| Example 6 | Compound 10 | 4.1 | 1.62 | 1.51 | blue |
| Example 7 | Compound 47 | 3.9 | 1.67 | 1.56 | blue |
| Example 8 | Compound 52 | 3.8 | 1.67 | 1.67 | blue |
| Comparative | Compound C1 | 4.3 | 1.11 | 1.08 | blue |
| Example 6 | | | | | |
| Comparative | Compound C2 | 4.4 | 1.23 | 0.88 | blue |
| Example 7 | | | | | |
| Comparative | Compound C3 | 4.9 | 0.91 | 0.61 | blue |
| Example 8 | | | | | |
| Comparative | Compound C4 | 5.1 | 0.94 | 0.58 | blue |
| Example 9 | | | | | |
| Comparative | Compound C5 | 5.4 | 74 | 46 | blue |
| Example 10 | | | | | |

Referring to Table 3, in the organic light-emitting devices of Examples introducing heterogeneous dopants, the driving voltage was reduced and the luminous efficiency was improved. Additionally, the color conversion efficiency ratio was improved by 1.55 times or more compared to that from Comparative Example 1, and the life-span ratio was also improved by 1.35 times or more compared to that from Comparative Example 1.

Additionally, the organic light-emitting devices of Comparative Examples 6 to 10 did not include the heterocyclic compound of the present disclosure as a host material for the emission layer, and also provided higher driving voltage and lower color conversion efficiency ratio and lifespan ratio that those from the organic light-emitting devices of Examples.

What is claimed is:

1. A heterocyclic compound represented by Chemical Formula 1: 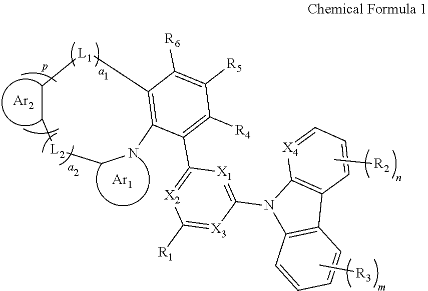
wherein, in Chemical Formula 1, Ar₁ is a substituted or unsubstituted C₂-C₆₀ nitrogen-containing heteroaryl ring;
Ar₂ is a substituted or unsubstituted C₆-C₆₀ aryl ring or a substituted or unsubstituted C₂-C₆₀ heteroaryl ring;
X₁ to X₄ are each independently N or CR₇, provided that at least two of X¹ to X₃ are N;
R₁ to R₇ are each independently hydrogen, deuterium, halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₁-C₆₀ heterocycloalkenyl group, a substituted or unsubstituted C₆-C₆₀ aryl group, a substituted or unsubstituted C₆-C₆₀ alkyl aryl group, a substituted or unsubstituted C₆-C₆₀ aryl alkyl group, a substituted or unsubstituted C₂-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₆-C₆₀ aryloxy group, a substituted or unsubstituted C₆-C₆₀ arylthio group, a substituted or unsubstituted C₈-C₆₀ condensed polycyclic group, —SiRR′R″, —P(═O) RR′, —NRR′, —BRR′, —C(═O) R or —S(═O)₂R, or
two or more adjacent groups of R₁ to R₇ are optionally combined with each other to form a substituted or unsubstituted C₃-C₆₀ cycloalkyl ring, a substituted or unsubstituted C₅-C₆₀ cycloalkenyl ring, a substituted or unsubstituted C₃-C₆₀ heterocycloalkyl ring, a substituted or unsubstituted C₁-C₆₀ heterocycloalkenyl ring, a substituted or unsubstituted C₆-C₆₀ aryl ring or a substituted or unsubstituted C₂-C₆₀ heteroaryl ring;
R, R′ and R″ are each independently hydrogen, deuterium, halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₁-C₆₀ heterocycloalkenyl group, a substituted or unsubstituted C₆-C₆₀ aryl group, a substituted or unsubstituted C₆-C₆₀ alkyl aryl group, a substituted or unsubstituted C₆-C₆₀ aryl alkyl group, a substituted or unsubstituted C₂-C₆₀ heteroaryl group, a substituted or unsubstituted C₂-C₆₀ alkyl heteroaryl group, or a substituted or unsubstituted C₂-C₆₀ heteroaryl alkyl group, a substituted or unsubstituted C₆-C₆₀ aryloxy group, a substituted or unsubstituted C₆-C₆₀ arylthio group, or a substituted or unsubstituted C₈-C₆₀ condensed polycyclic group;
L₁ and L₂ are each independently a direct bond, O, S, NR₈;
R₈ is a substituted or unsubstituted C₆-C₆₀ aryl group, a substituted or unsubstituted C₆-C₆₀ alkyl aryl group, a substituted or unsubstituted C₆-C₆₀ aryl alkyl group, a substituted or unsubstituted C₂-C₆₀ heteroaryl group, a substituted or unsubstituted C₂-C₆₀ alkyl heteroaryl group, a substituted or unsubstituted C₂-C₆₀ heteroaryl alkyl group, or is combined with Ar₁, Ar₂ or R₆ to form a substituted or unsubstituted C₂-C₆₀ nitrogen-containing heteroaryl ring;
m, a₁ and a₂ are each independently an integer of 0 to 4, and n is an integer of 0 to 3;
p is an integer of 1 to 3; and
when n is 2 or more, each R₂ is different or the same,
when m is 2 or more, each R₃ is different or the same,
when p is 2 or more, each Ar₂ is different or the same,
when a₁ is 2 or more, each L₁ are different or the same, or
when a₂ is 2 or more, each L₂ is different or the same.

2. The heterocyclic compound of claim 1, wherein the heterocyclic compound is represented by Chemical Formula 1-1: 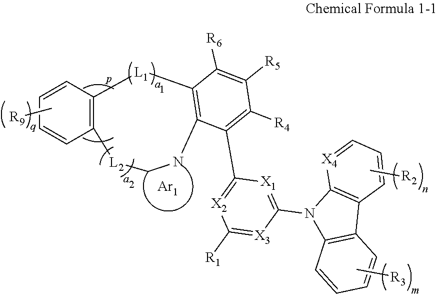
wherein, in Chemical Formula 1-1, Ar₁, X₁ to X₄, R₁ to R₆, L₁, L₂, n, m, p, a₁ and a₂ are the same as those defined in claim 1;
R₉ is hydrogen, deuterium, halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₁-C₆₀ heterocycloalkenyl group, a substituted or unsubstituted C₆-C₆₀ aryl group, a substituted or unsubstituted C₆-C₆₀ alkyl aryl group, a substituted or unsubstituted C₆-C₆₀ aryl alkyl group, a substituted or unsubstituted C₂-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₆-C₆₀ aryloxy group, a substituted or unsubstituted C₆-C₆₀ arylthio group, a substituted or unsubstituted C₈-C₆₀ condensed polycyclic group, —SiRR′R″, —P(═O) RR′, —NRR′, —BRR′, —C(═O) R or —S(═O)₂R, or
when q is 2 or more, two or more adjacent groups R₉ are optionally combined with each other to form a substituted or unsubstituted C₃-C₆₀ cycloalkyl ring, a substituted or unsubstituted C₅-C₆₀ cycloalkenyl ring, a substituted or unsubstituted C₃-C₆₀ heterocycloalkyl ring, a substituted or unsubstituted C₁-C₆₀ heterocycloalkenyl ring, a substituted or unsubstituted C₆-C₆₀ aryl ring or a substituted or unsubstituted C₂-C₆₀ heteroaryl ring;
R, R′ and R″ are each independently hydrogen, deuterium, halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₁-C₆₀ heterocycloalkenyl group, a substituted or unsubstituted C₆-C₆₀ aryl group, a substituted or unsubstituted C₆-C₆₀ alkyl aryl group, a substituted or unsubstituted C₆-C₆₀ aryl alkyl group, a substituted or unsubstituted C₂-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₆-C₆₀ aryloxy group, a substituted or unsubstituted C₆-C₆₀ arylthio group, or a substituted or unsubstituted C₈-C₆₀ condensed polycyclic group; and
q is an integer of 0 to 4, and when q is 2 or more, each of R₉ is the same or different.

3. The heterocyclic compound of claim 1, wherein the heterocyclic compound is represented by Chemical Formula 1-2: 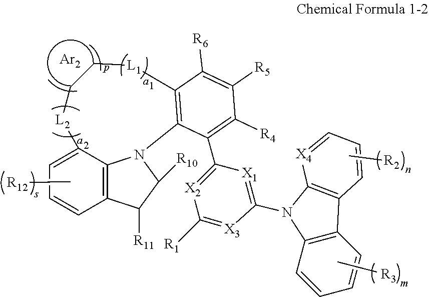
wherein, in Chemical Formula 1-2, Ar₂, X₁ to X₄, R₁ to R₆, L₁, L₂, n, m, p, a₁ and a₂ are the same as those defined in claim 1;
R₁₀ to R₁₂ are each independently hydrogen, deuterium, halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₁-C₆₀ heterocycloalkenyl group, a substituted or unsubstituted C₆-C₆₀ aryl group, a substituted or unsubstituted C₆-C₆₀ alkyl aryl group, a substituted or unsubstituted C₆-C₆₀ aryl alkyl group, a substituted or unsubstituted C₂-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₆-C₆₀ aryloxy group, a substituted or unsubstituted C₆-C₆₀ arylthio group, a substituted or unsubstituted C₈-C₆₀ condensed polycyclic group, —SiRR′R″, —P(═O) RR′, —NRR′, —BRR′, —C(═O) R or —S(═O)₂R, or
two or more adjacent ones of R₁₀ to R₁₂ are optionally combined with each other to form a substituted or unsubstituted C₃-C₆₀ cycloalkyl ring, a substituted or unsubstituted C₅-C₆₀ cycloalkenyl ring, a substituted or unsubstituted C₃-C₆₀ heterocycloalkyl ring, a substituted or unsubstituted C₁-C₆₀ heterocycloalkenyl ring, a substituted or unsubstituted C₆-C₆₀ aryl ring, or a substituted or unsubstituted C₂-C₆₀ heteroaryl ring;
R, R′ and R″ are each independently hydrogen, deuterium, halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₁-C₆₀ heterocycloalkenyl group, a substituted or unsubstituted C₆-C₆₀ aryl group, a substituted or unsubstituted C₆-C₆₀ alkyl aryl group, a substituted or unsubstituted C₆-C₆₀ aryl alkyl group, a substituted or unsubstituted C₂-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₆-C₆₀ aryloxy group, a substituted or unsubstituted C₆-C₆₀ arylthio group, or a substituted or unsubstituted C₈-C₆₀ condensed polycyclic group; and
s is an integer of 0 to 3, and when s is 2 or more, multiple R₁₂ are the same as or different from each other.

4. The heterocyclic compound of claim 1, wherein the heterocyclic compound is represented by any one of Chemical Formulae 1-3 to 1-6: 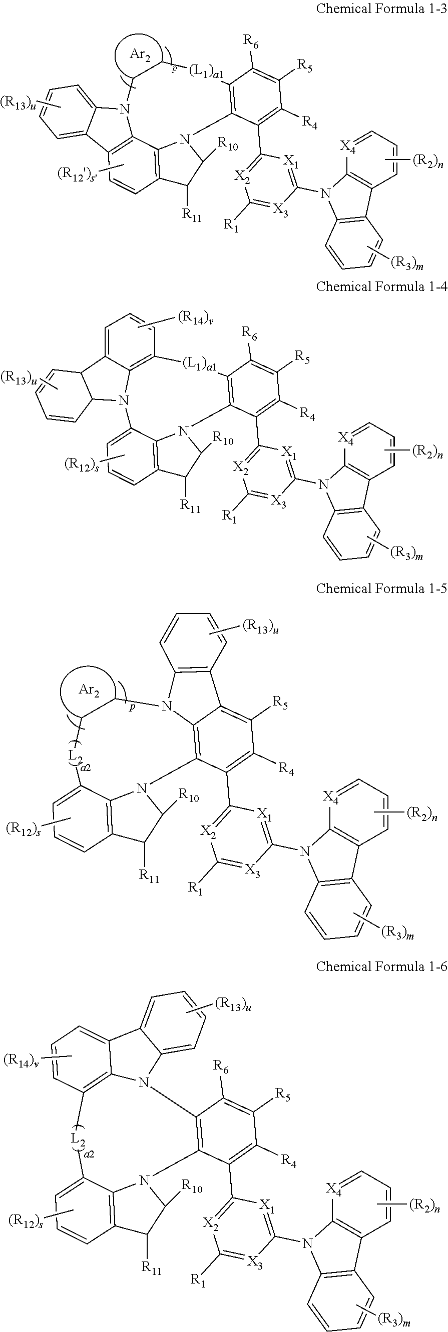
wherein, in Chemical Formulae 1-3 to 1-6, Ar₂, X₁ to X₄, R₁ to R₆, L₁, n, m, p, a₁, and a₂ are the same as those defined in claim 1;
R₁₀ to R₁₄ and R₁₂′ are each independently hydrogen, deuterium, halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₁-C₆₀ heterocycloalkenyl group, a substituted or unsubstituted C₆-C₆₀ aryl group, a substituted or unsubstituted C₆-C₆₀ alkyl aryl group, a substituted or unsubstituted C₆-C₆₀ aryl alkyl group, a substituted or unsubstituted C₂-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₆-C₆₀ aryloxy group, a substituted or unsubstituted C₆-C₆₀ arylthio group, a substituted or unsubstituted C₈-C₆₀ condensed polycyclic group, —SiRR′R″, —P(═O)RR′, —NRR′, —BRR′, —C(═O) R or —S(═O)₂R, or
two or more adjacent ones of R₁₀ to R₁₄ and R₁₂′ are optionally combined with each other to form a substituted or unsubstituted C₃-C₆₀ cycloalkyl ring, a substituted or unsubstituted C₅-C₆₀ cycloalkenyl ring, a substituted or unsubstituted C₃-C₆₀ heterocycloalkyl ring, a substituted or unsubstituted C₁-C₆₀ heterocycloalkenyl ring, a substituted or unsubstituted C₆-C₆₀ aryl ring, or a substituted or unsubstituted C₂-C₆₀ heteroaryl ring;
R, R′ and R″ are each independently hydrogen, deuterium, halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₁-C₆₀ heterocycloalkenyl group, a substituted or unsubstituted C₆-C₆₀ aryl group, a substituted or unsubstituted C₆-C₆₀ alkyl aryl group, a substituted or unsubstituted C₆-C₆₀ aryl alkyl group, a substituted or unsubstituted C₂-C₆₀ heteroaryl group, a substituted or unsubstituted C₂-C₆₀ alkyl heteroaryl group, or a substituted or unsubstituted C₂-C₆₀ heteroaryl alkyl group, a substituted or unsubstituted C₆-C₆₀ aryloxy group, a substituted or unsubstituted C₆-C₆₀ arylthio group, or a substituted or unsubstituted C₈-C₆₀ condensed polycyclic group;
s and v are each independently an integer of 0 to 3, u is an integer of 0 to 4, and s′ is 0, 1, or 2;
when s, v, and u are each independently 2 or more, a plurality of each of R₁₂ R₁₃, and R₁₄ are the same as or different from each other; and
when s′ is 2, a plurality of R₁₂′ are the same as or different from each other.

5. The heterocyclic compound of claim 1, wherein, in Chemical Formula 1, p is 1 or 2.

6. The heterocyclic compound of claim 1, wherein the heterocyclic compound is represented by any one of Chemical Formulae 1-7 to 1-9: 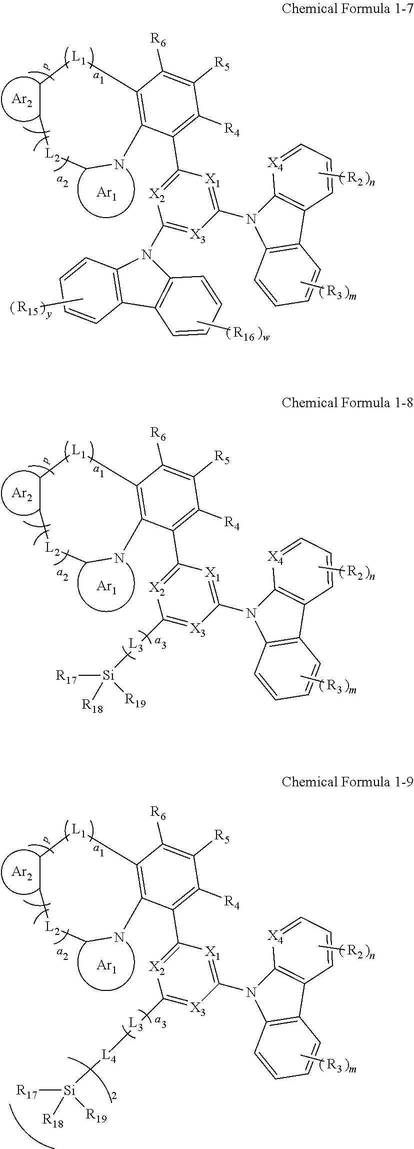
wherein, in Chemical Formulae 1-7 to 1-9, Ar₁, Ar₂, X₁ to X₄, R₂ to R₆, L₁, L₂, n, m, p, a₁ and a₂ are the same as those defined in claim 1;
R₁₅ to R₁₉ are each independently hydrogen, deuterium, halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₁-C₆₀ heterocycloalkenyl group, a substituted or unsubstituted C₆-C₆₀ aryl group, a substituted or unsubstituted C₆-C₆₀ alkyl aryl group, a substituted or unsubstituted C₆-C₆₀ aryl alkyl group, a substituted or unsubstituted C₂-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₆-C₆₀ aryloxy group, a substituted or unsubstituted C₆-C₆₀ arylthio group, a substituted or unsubstituted C₈-C₆₀ condensed polycyclic group, —SiRR′R″, —P(═O) RR′, —NRR′, —BRR′, —C(═O) R or —S(═O)₂R, or
two or more adjacent ones of R₁₅ to R₁₉ are optionally combined with each other to form a substituted or unsubstituted C₃-C₆₀ cycloalkyl ring, a substituted or unsubstituted C₅-C₆₀ cycloalkenyl ring, a substituted or unsubstituted C₃-C₆₀ heterocycloalkyl ring, a substituted or unsubstituted C₁-C₆₀ heterocycloalkenyl ring, a substituted or unsubstituted C₆-C₆₀ aryl ring, or a substituted or unsubstituted C₂-C₆₀ heteroaryl ring,
R, R′ and R″ are each independently hydrogen, deuterium, halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₁-C₆₀ heterocycloalkenyl group, a substituted or unsubstituted C₆-C₆₀ aryl group, a substituted or unsubstituted C₆-C₆₀ alkyl aryl group, a substituted or unsubstituted C₆-C₆₀ aryl alkyl group, a substituted or unsubstituted C₂-C₆₀ heteroaryl group, a substituted or unsubstituted C₂-C₆₀ alkyl heteroaryl group, substituted or unsubstituted C₂-C₆₀ heteroaryl alkyl group, a substituted or unsubstituted C₆-C₆₀ aryloxy group, a substituted or unsubstituted C₆-C₆₀ arylthio group, or a substituted or unsubstituted C₈-C₆₀ condensed polycyclic group;
w and y are each independently an integer of 0 to 4, and when w and y are each independently 2 or more, a plurality of each of R₁₅ and R₁₆ are the same as or different from each other;
L₃ is a direct bond, a substituted or unsubstituted C₆-C₆₀ arylene group, or a substituted or unsubstituted C₂-C₆₀ heteroarylene group;
a₃ is an integer of 0 to 4, and when a₃ is 2 or more, a plurality of L₃ are the same as or different from each other;
L₄ is a trivalent organic group in which any two hydrogens are removed from a substituted or unsubstituted C₆-C₆₀ aryl group, a substituted or unsubstituted C₆-C₆₀ alkyl aryl group, a substituted or unsubstituted C₆-C₆₀ aryl alkyl group, a substituted or unsubstituted C₂-C₆₀ heteroaryl group, a substituted or unsubstituted C₂-C₆₀ alkyl heteroaryl group, or a substituted or unsubstituted C₂-C₆₀ heteroaryl alkyl group; and
two of each of R₁₇ to R₁₉ in Chemical Formula 1-9 are the same as or different from each other.

7. The heterocyclic compound of claim 1, wherein, in Chemical Formula 1, X₁ to X₃ are N; or X₁ and X₂ are N, and X₃ is CR₇.

8. The heterocyclic compound of claim 1, wherein, in Chemical Formula 1, at least one of a₁ and a₂ is not 0.

9. The heterocyclic compound of claim 1, wherein the heterocyclic compound comprises at least one of compounds represented by chemical formulae below:

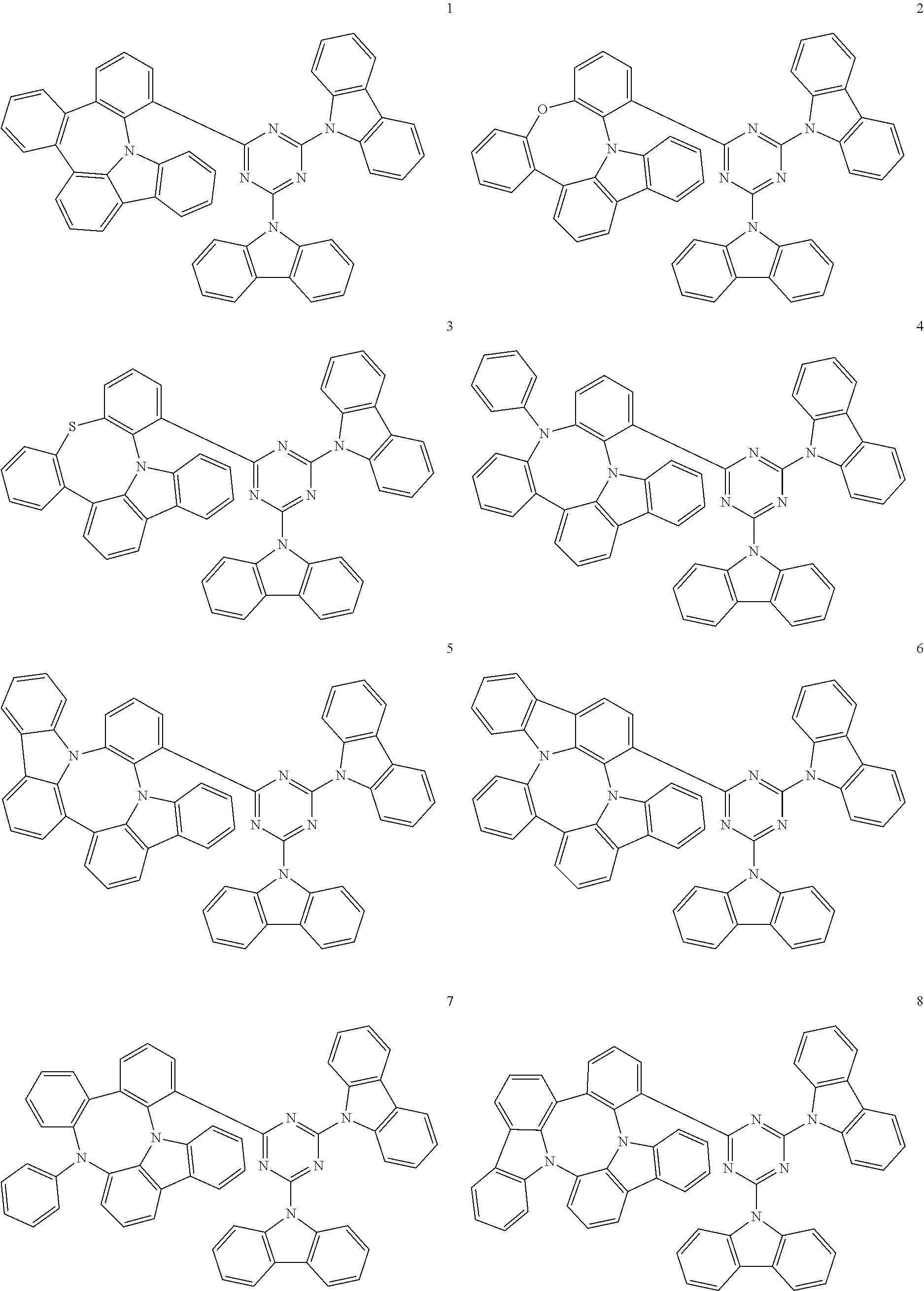

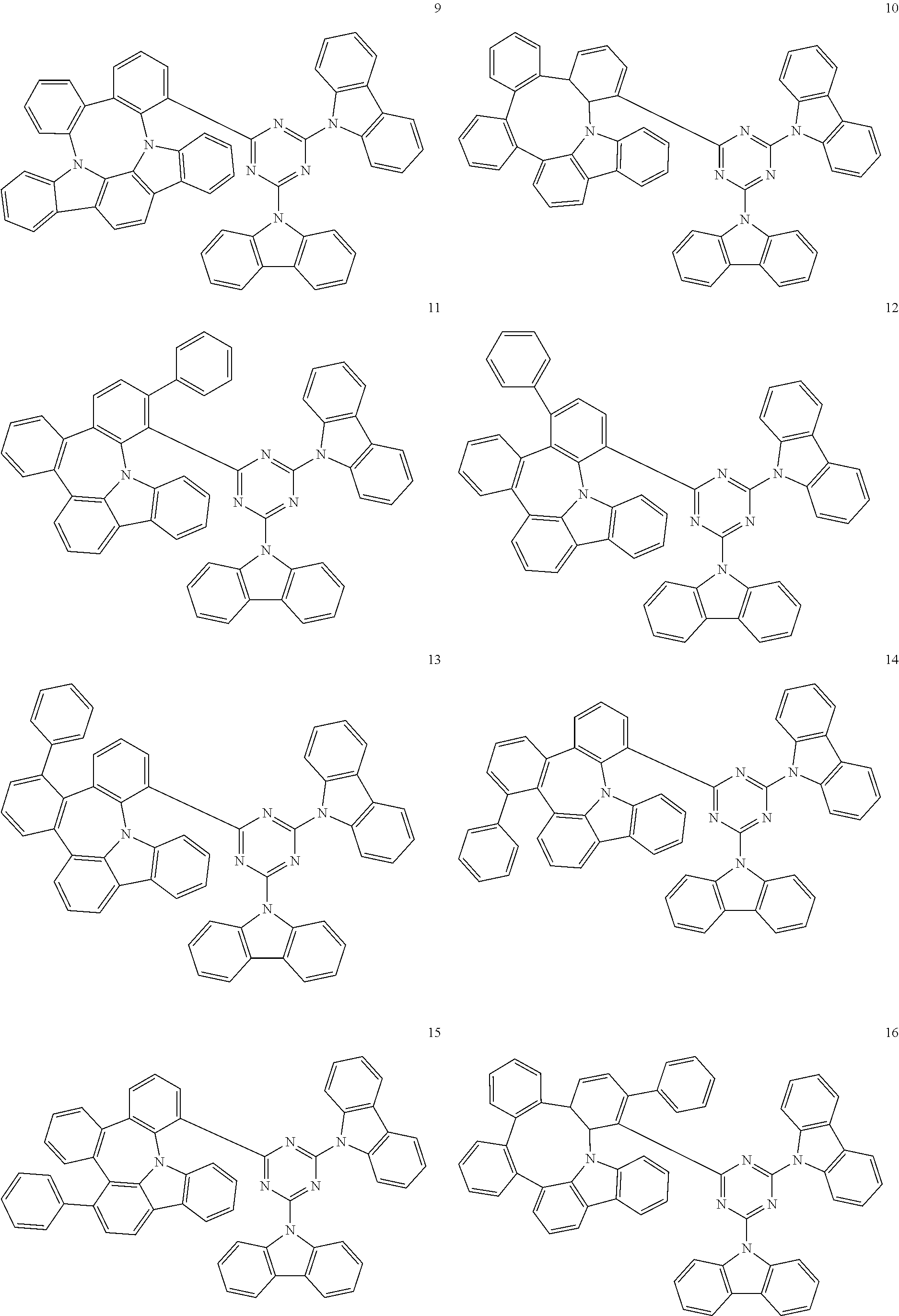

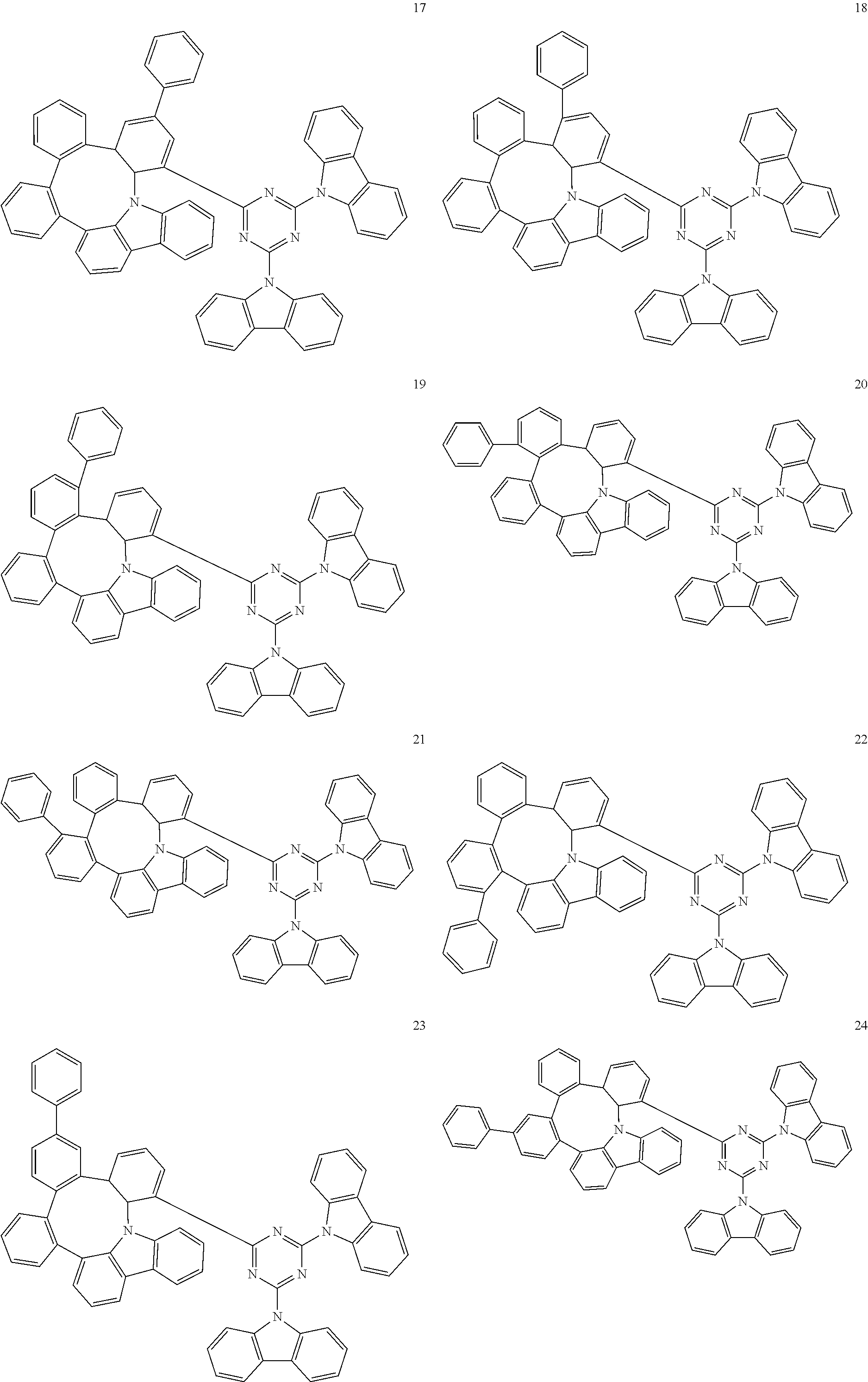

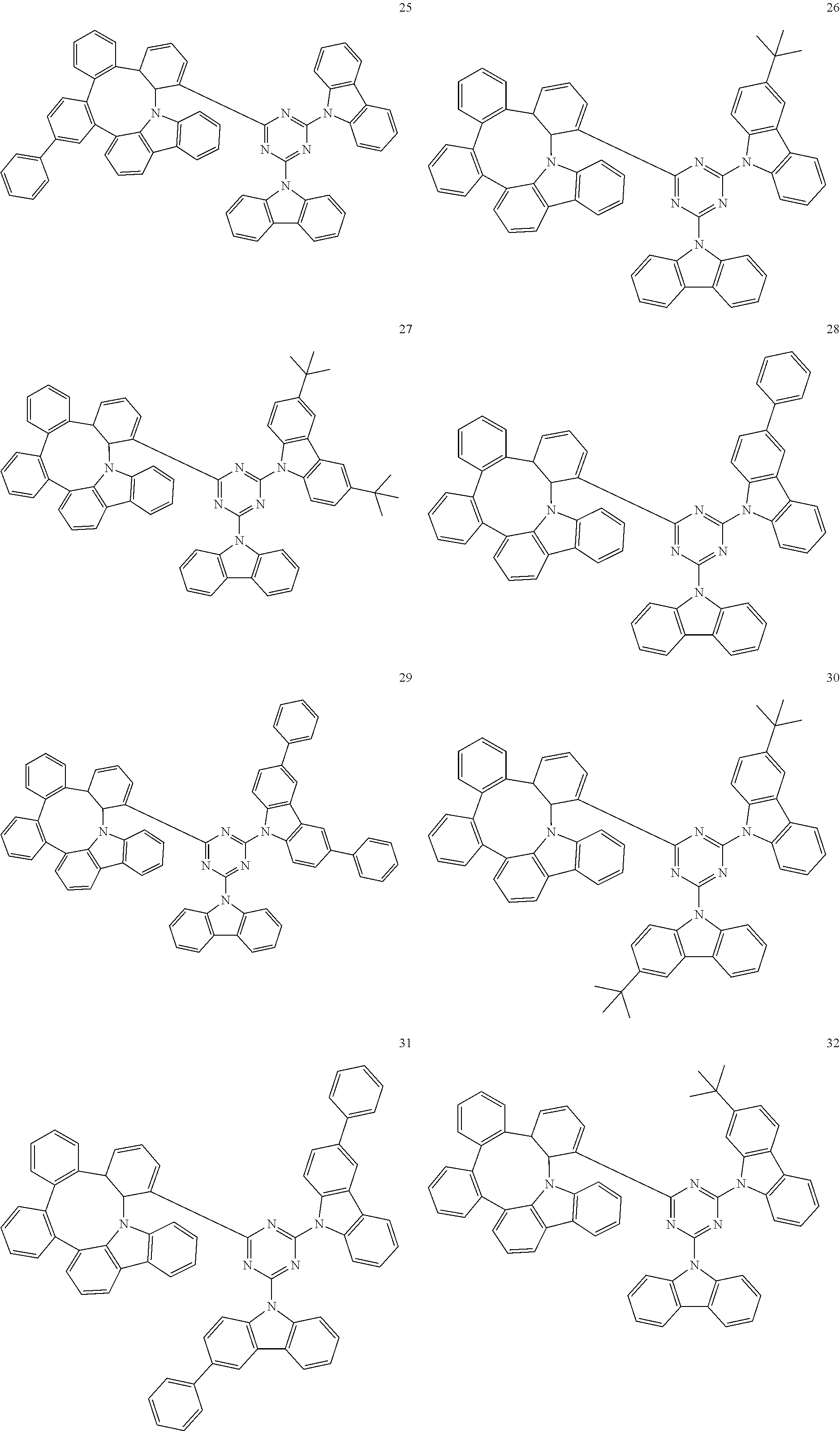

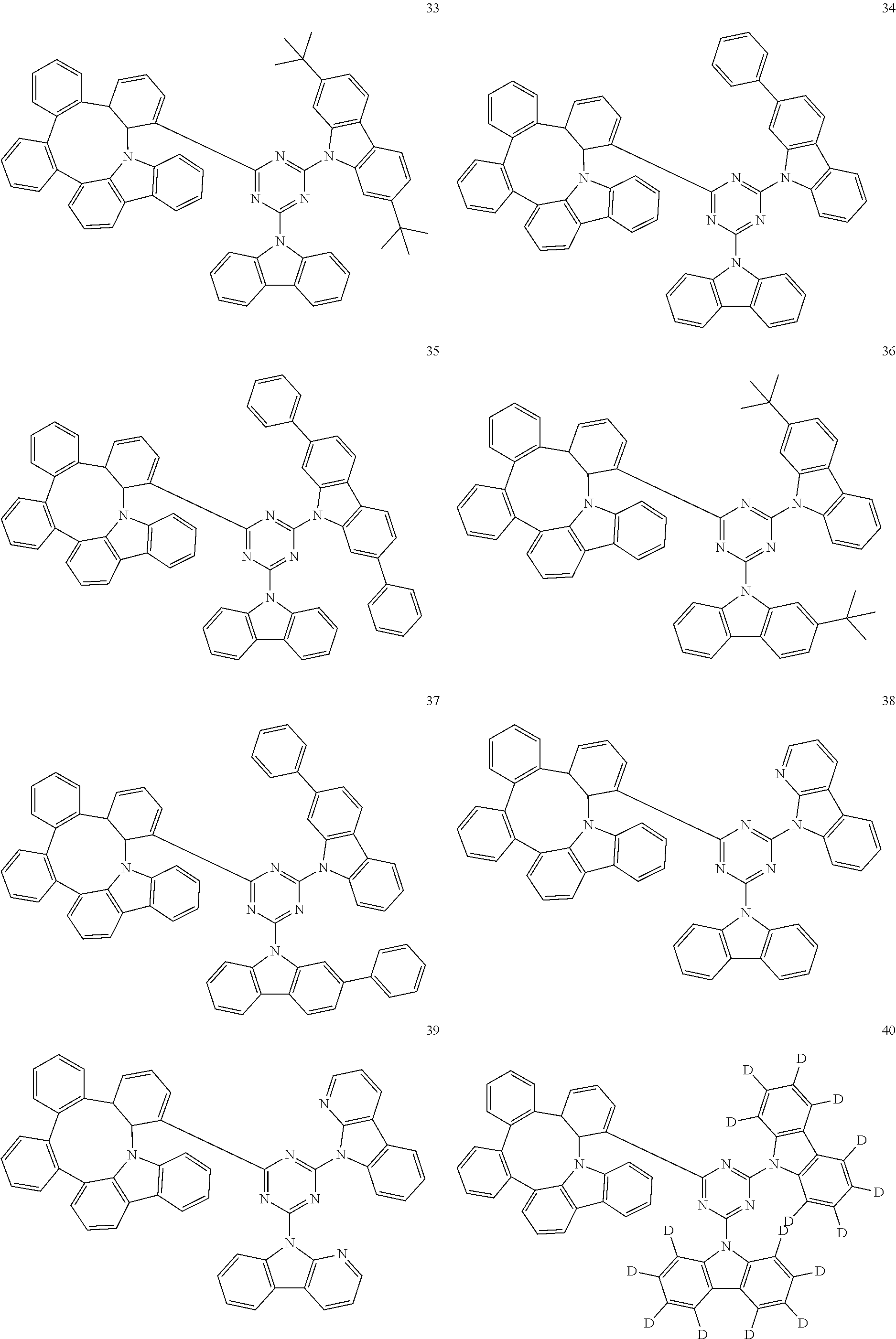

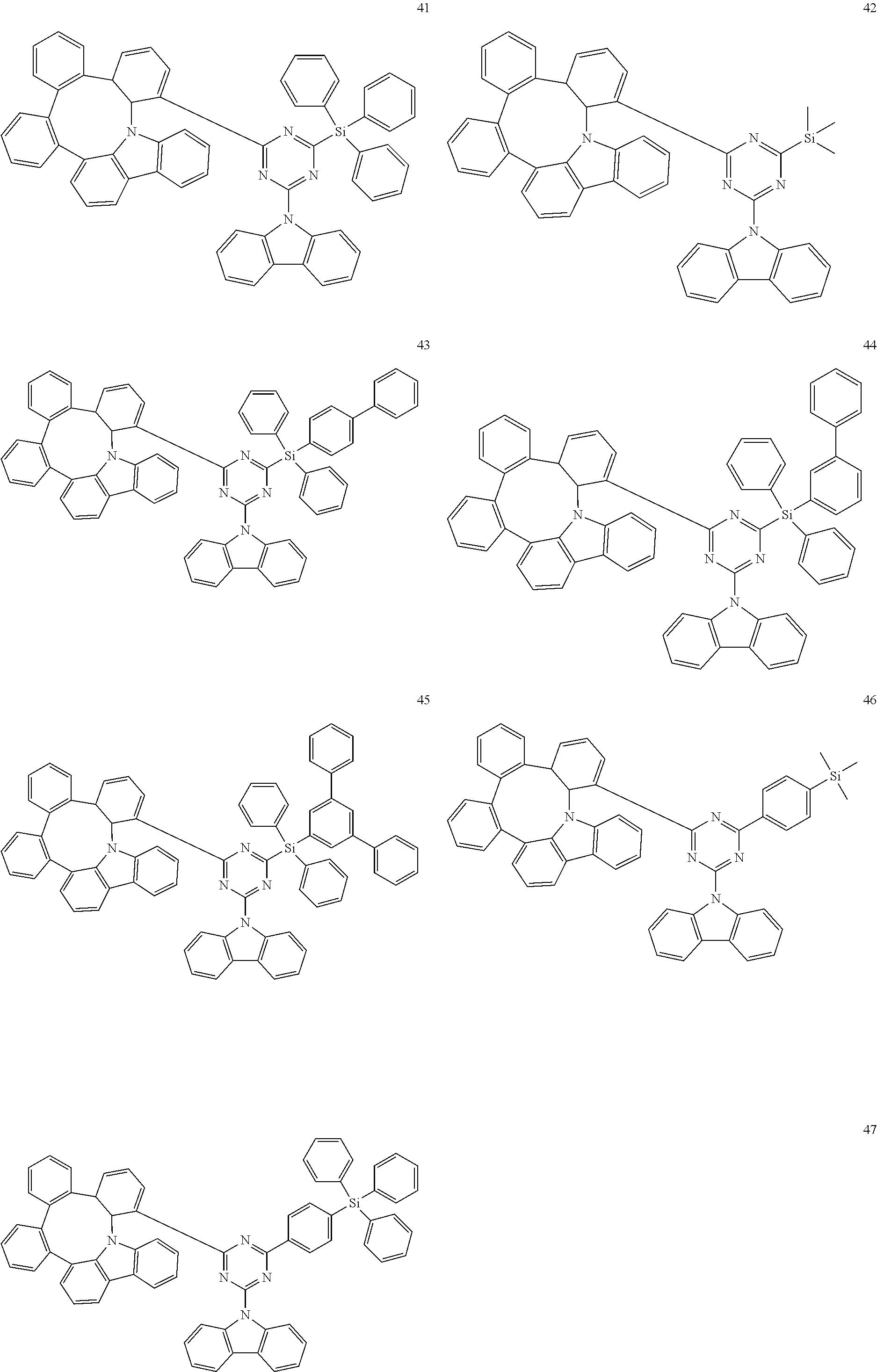

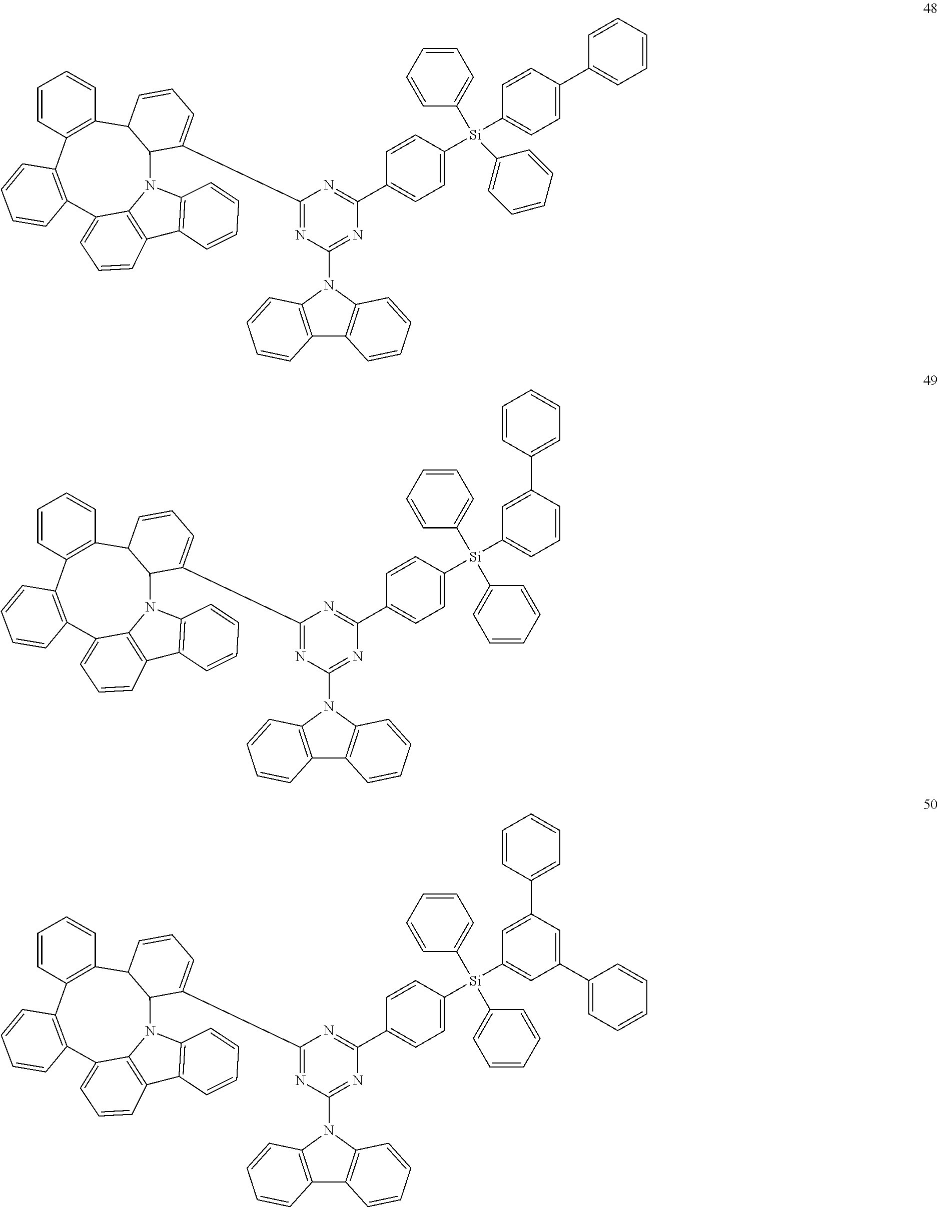 [SMILES: C1=CC2c3ccccc3-c3ccccc3-c3cccc4c5ccccc5n(c34)C2C(c2nc(-c3ccc([Si](c4ccccc4)(c4ccccc4)c4ccc(-c5ccccc5)cc4)cc3)nc(-n3c4ccccc4c4ccccc43)n2)=C1 ; C1=CC2c3ccccc3-c3ccccc3-c3cccc4c5ccccc5n(c34)C2C(c2nc(-c3ccc([Si](c4ccccc4)(c4ccccc4)c4cccc(-c5ccccc5)c4)cc3)nc(-n3c4ccccc4c4ccccc43)n2)=C1 ; C1=CC2c3ccccc3-c3ccccc3-c3cccc4c5ccccc5n(c34)C2C(c2nc(-c3ccc([Si](c4ccccc4)(c4ccccc4)c4cc(-c5ccccc5)cc(-c5ccccc5)c4)cc3)nc(-n3c4ccccc4c4ccccc43)n2)=C1]

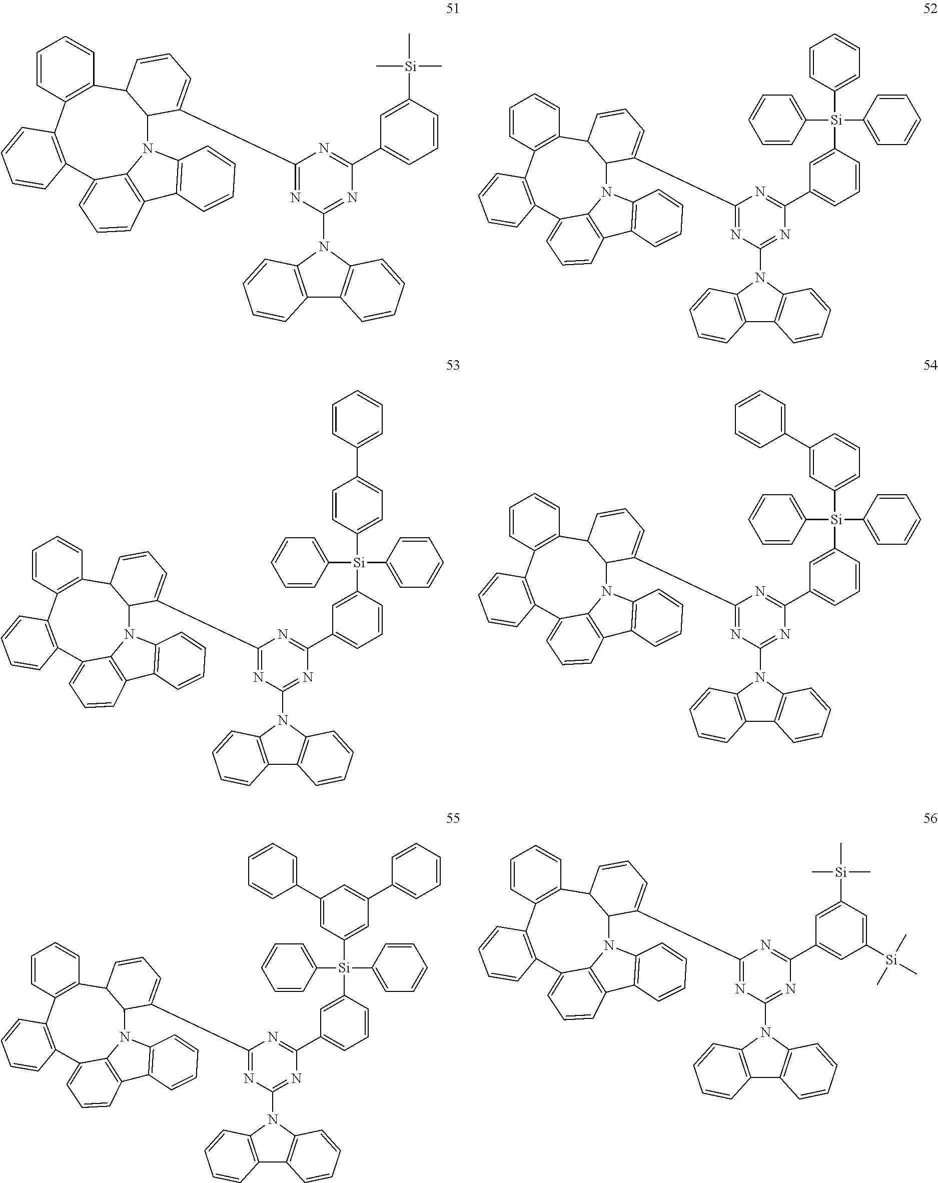

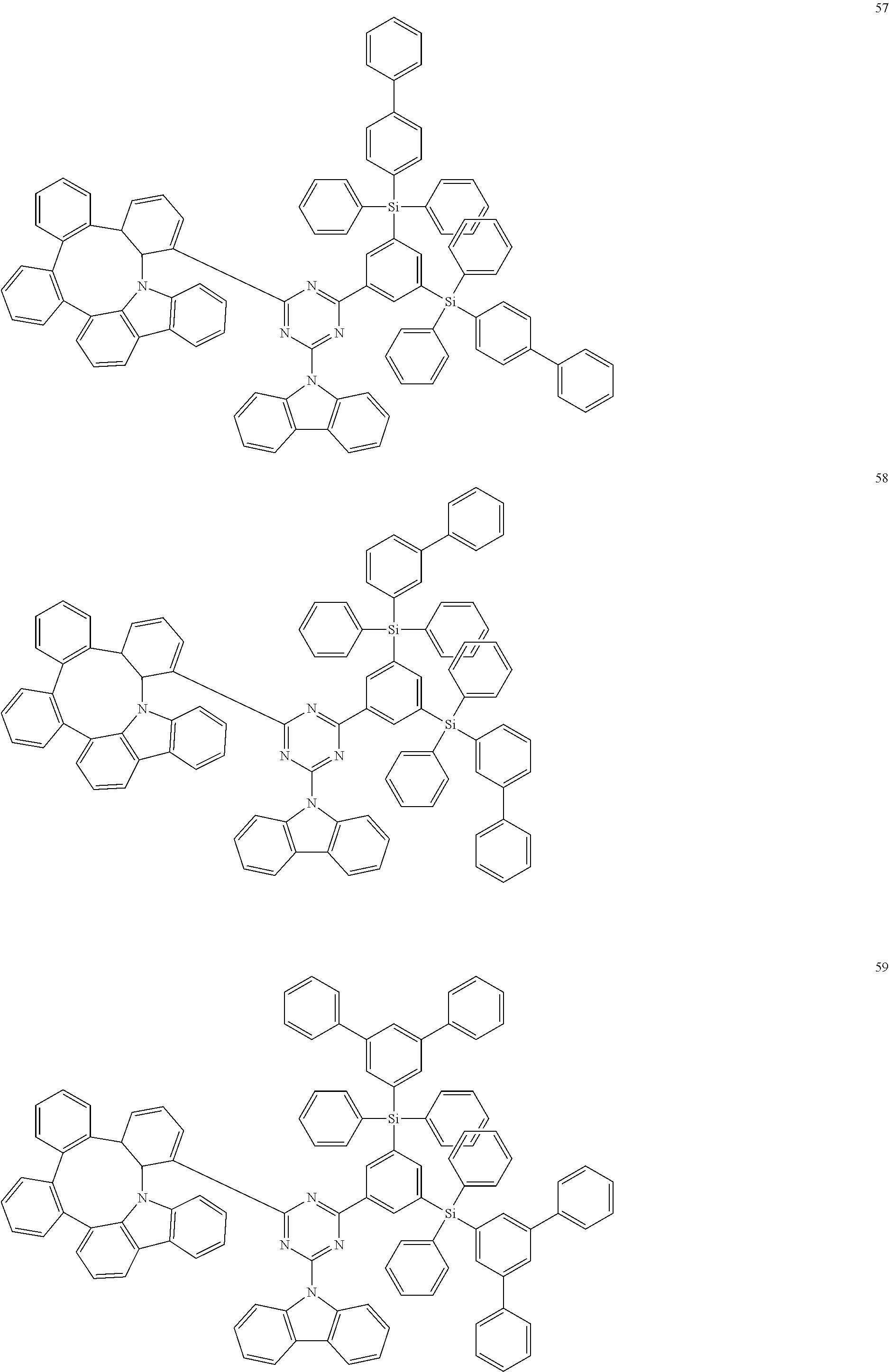

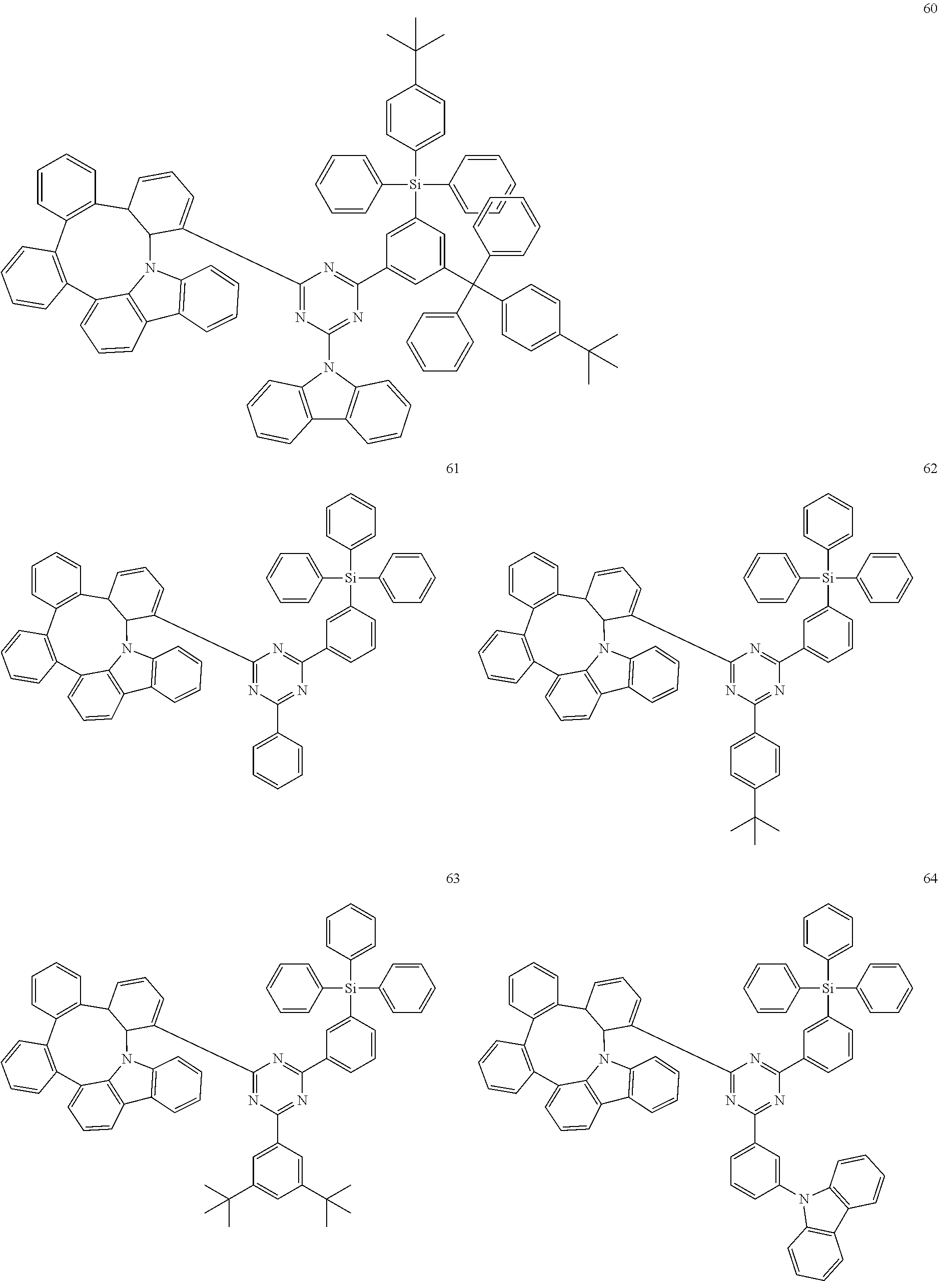

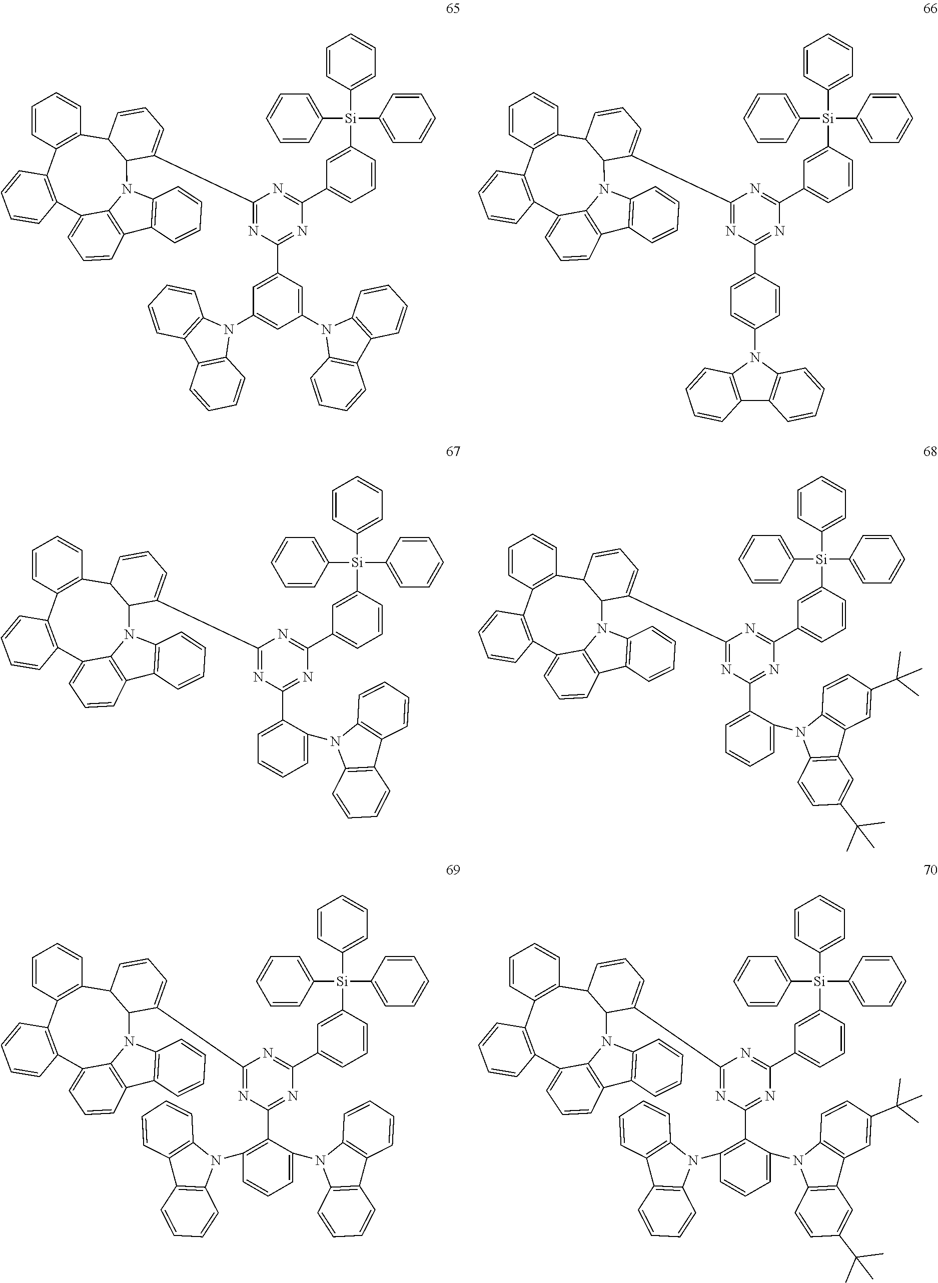

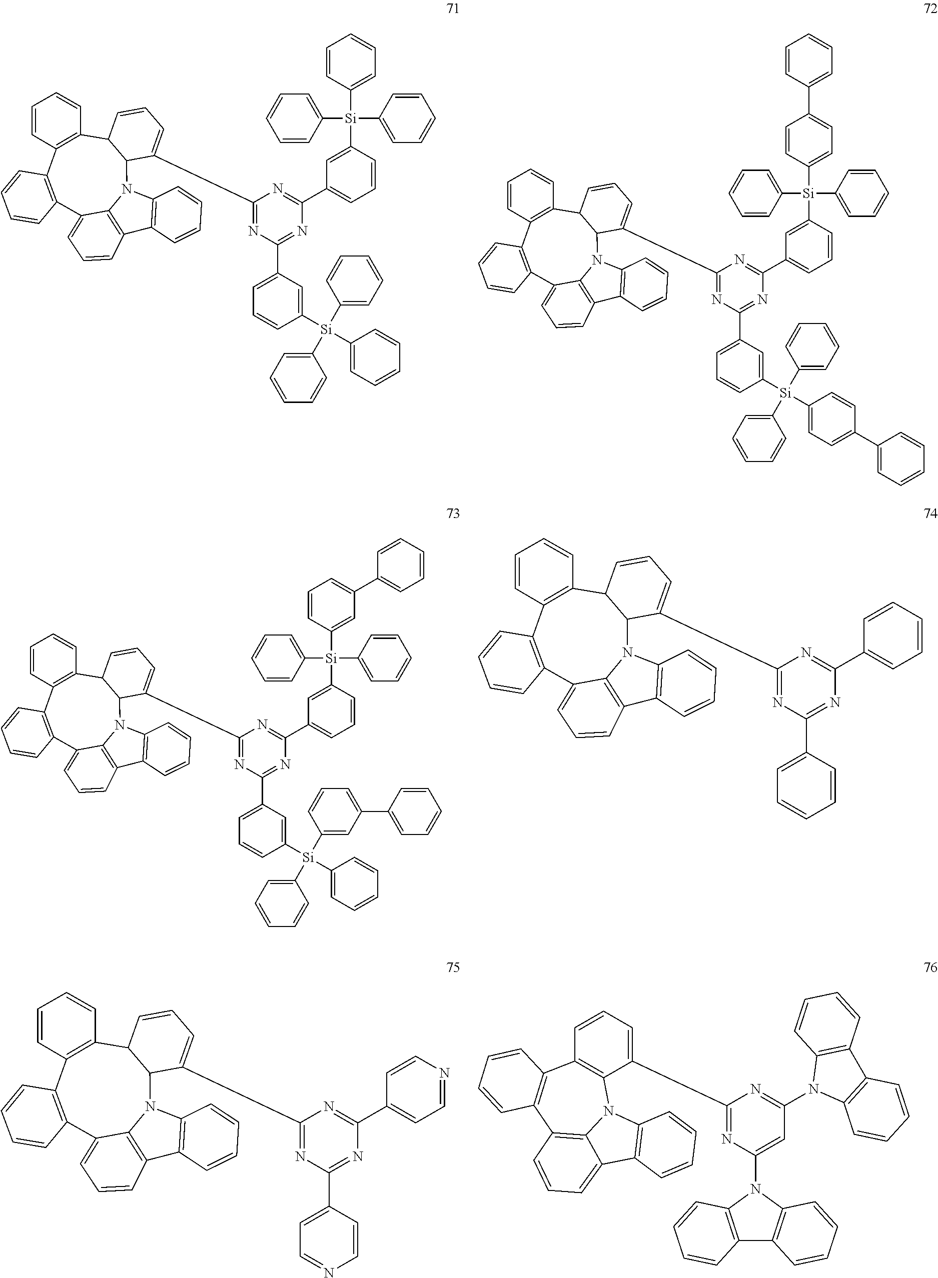

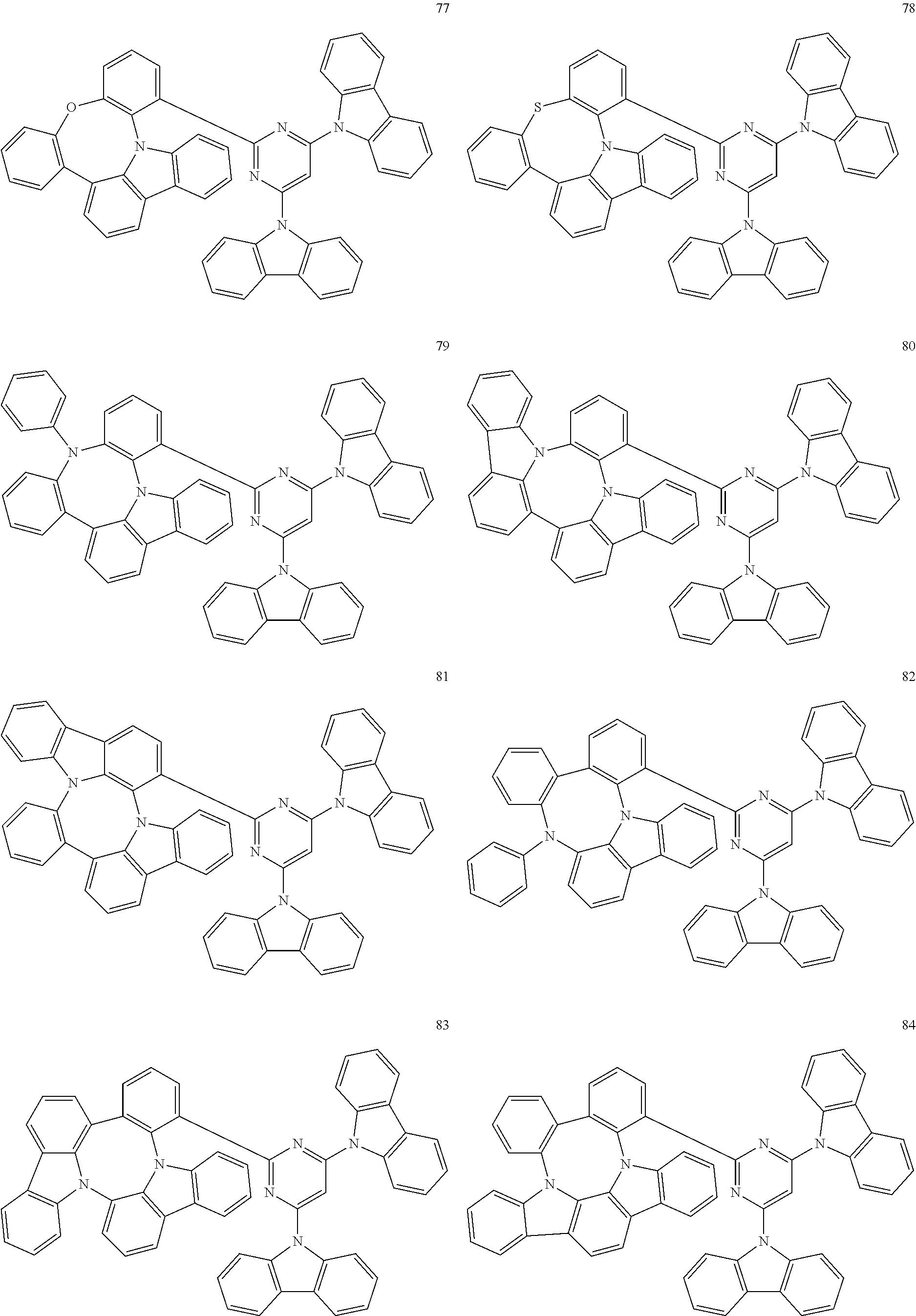

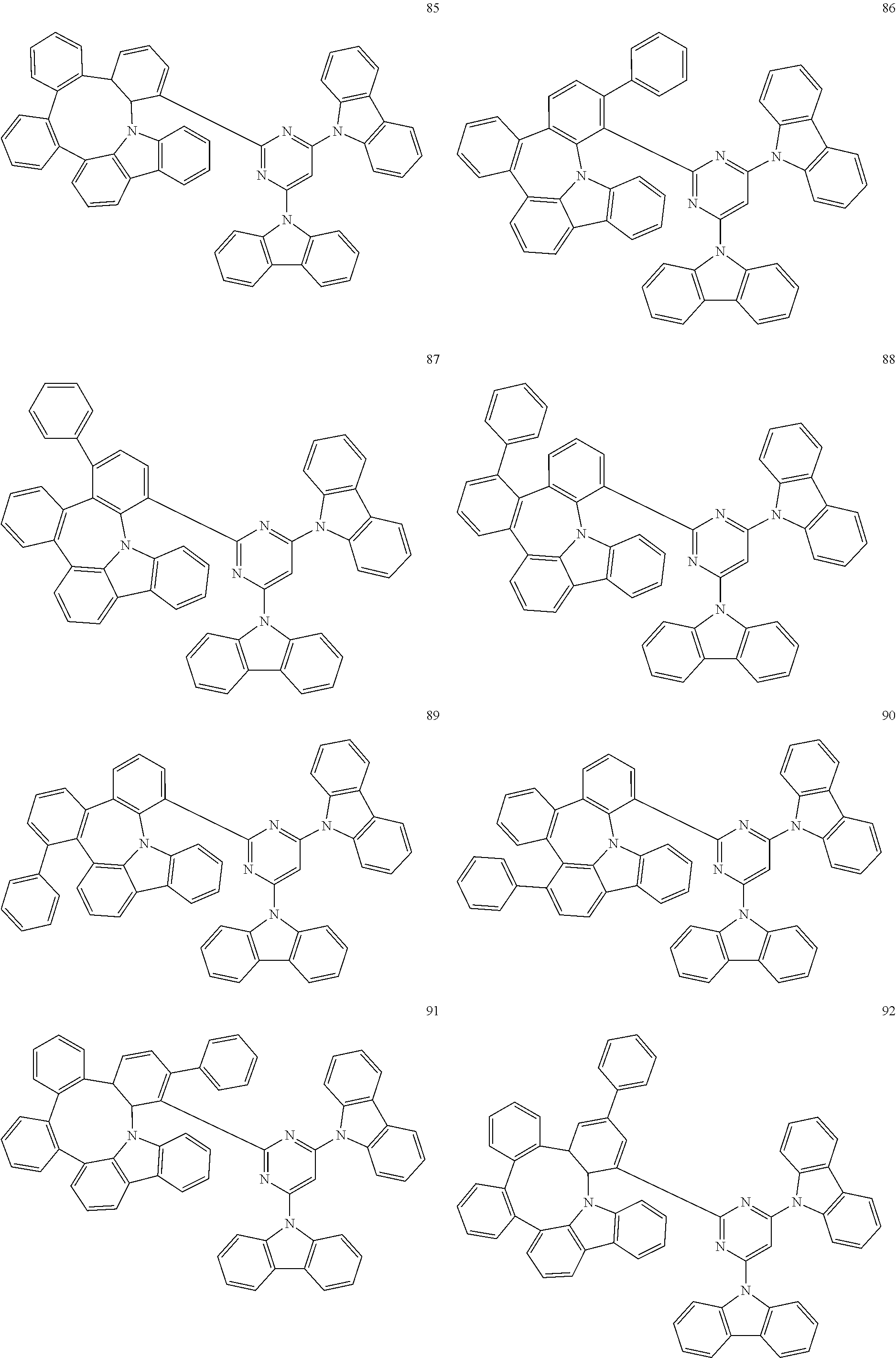

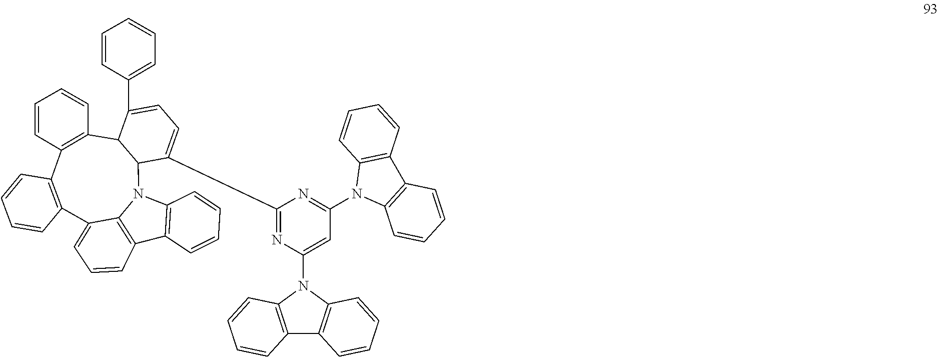 [SMILES: C1=C(c2ccccc2)C2c3ccccc3-c3ccccc3-c3cccc4c5ccccc5n(c34)C2C(c2nc(-n3c4ccccc4c4ccccc43)cc(-n3c4ccccc4c4ccccc43)n2)=C1]

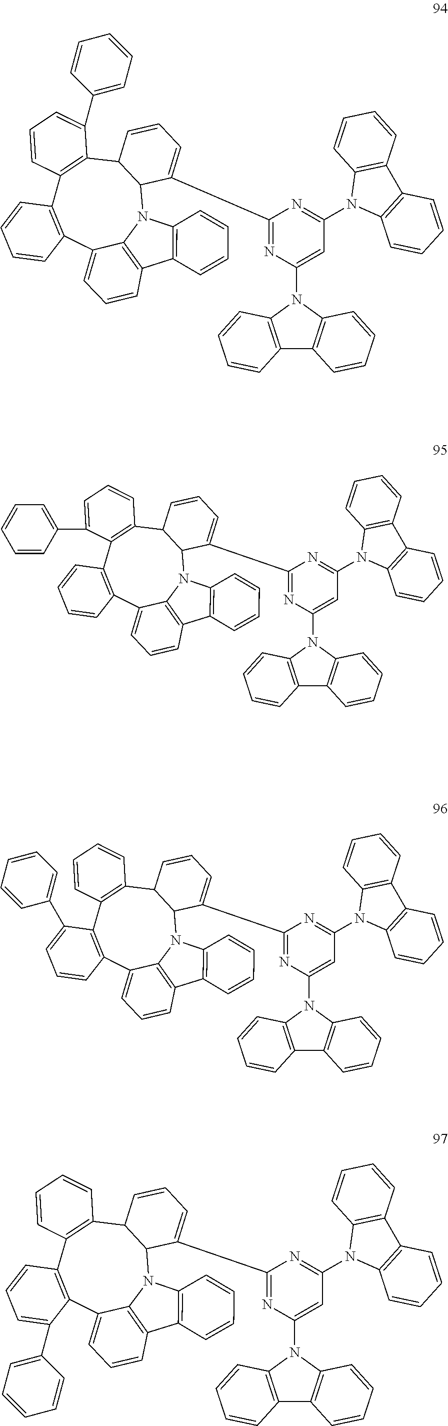 [SMILES: C1=CC2c3c(-c4ccccc4)cccc3-c3ccccc3-c3cccc4c5ccccc5n(c34)C2C(c2nc(-n3c4ccccc4c4ccccc43)cc(-n3c4ccccc4c4ccccc43)n2)=C1 ; C1=CC2c3cccc(-c4ccccc4)c3-c3ccccc3-c3cccc4c5ccccc5n(c34)C2C(c2nc(-n3c4ccccc4c4ccccc43)cc(-n3c4ccccc4c4ccccc43)n2)=C1 ; C1=CC2c3ccccc3-c3c(-c4ccccc4)cccc3-c3cccc4c5ccccc5n(c34)C2C(c2nc(-n3c4ccccc4c4ccccc43)cc(-n3c4ccccc4c4ccccc43)n2)=C1 ; C1=CC2c3ccccc3-c3cccc(-c4ccccc4)c3-c3cccc4c5ccccc5n(c34)C2C(c2nc(-n3c4ccccc4c4ccccc43)cc(-n3c4ccccc4c4ccccc43)n2)=C1]

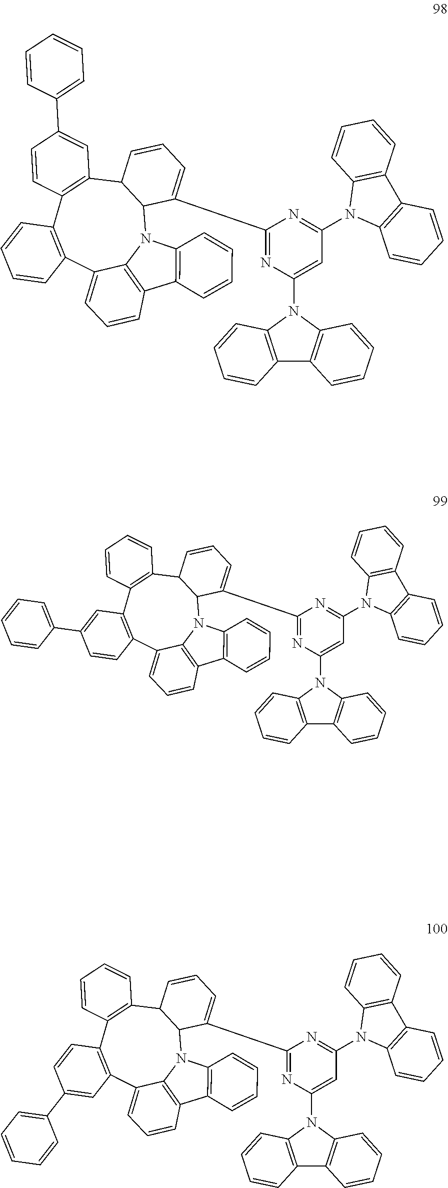 [SMILES: C1=CC2c3cc(-c4ccccc4)ccc3-c3ccccc3-c3cccc4c5ccccc5n(c34)C2C(c2nc(-n3c4ccccc4c4ccccc43)cc(-n3c4ccccc4c4ccccc43)n2)=C1 ; C1=CC2c3ccccc3-c3cc(-c4ccccc4)ccc3-c3cccc4c5ccccc5n(c34)C2C(c2nc(-n3c4ccccc4c4ccccc43)cc(-n3c4ccccc4c4ccccc43)n2)=C1 ; C1=CC2c3ccccc3-c3ccc(-c4ccccc4)cc3-c3cccc4c5ccccc5n(c34)C2C(c2nc(-n3c4ccccc4c4ccccc43)cc(-n3c4ccccc4c4ccccc43)n2)=C1]

10. A light-emitting device, comprising:
a first electrode;
a second electrode; and
an intermediate layer disposed between the first electrode and the second electrode, the intermediate layer including a heterocyclic compound represented by Chemical Formula 1: 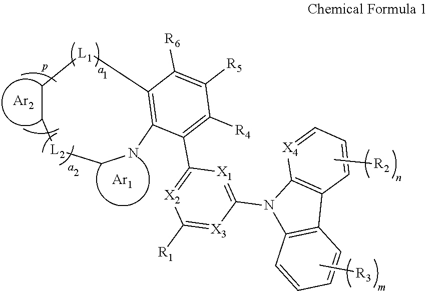
wherein, in Chemical Formula 1, Ar₁ is a substituted or unsubstituted C₂-C₆₀ nitrogen-containing heteroaryl ring;
Ar₂ is a substituted or unsubstituted C₆-C₆₀ aryl ring or a substituted or unsubstituted C₂-C₆₀ heteroaryl ring;
X₁ to X₄ are each independently N or CR₇, provided that at least two of X¹ to X₃ are N;
R₁ to R₇ are each independently hydrogen, deuterium, halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₁-C₆₀ heterocycloalkenyl group, a substituted or unsubstituted C₆-C₆₀ aryl group, a substituted or unsubstituted C₆-C₆₀ alkyl aryl group, a substituted or unsubstituted C₆-C₆₀ aryl alkyl group, a substituted or unsubstituted C₂-C₆₀ heteroaryl group, a substituted or unsubstituted C₂-C₆₀ alkyl heteroaryl group, or a substituted or unsubstituted C₂-C₆₀ heteroaryl alkyl group, a substituted or unsubstituted C₆-C₆₀ aryloxy group, a substituted or unsubstituted C₆-C₆₀ arylthio group, a substituted or unsubstituted C₈-C₆₀ condensed polycyclic group, —SiRR′R″, —P(═O) RR′, —NRR′, —BRR′, —C(═O) R or —S(═O)₂R, or
two or more adjacent groups among R₁ to R₇ are optionally combined with each other to form a substituted or unsubstituted C₃-C₆₀ cycloalkyl ring, a substituted or unsubstituted C₅-C₆₀ cycloalkenyl ring, a substituted or unsubstituted C₃-C₆₀ heterocycloalkyl ring, a substituted or unsubstituted C₁-C₆₀ heterocycloalkenyl ring, a substituted or unsubstituted C₆-C₆₀ aryl ring or a substituted or unsubstituted C₂-C₆₀ heteroaryl ring;
R, R′ and R″ are each independently hydrogen, deuterium, halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₁-C₆₀ heterocycloalkenyl group, a substituted or unsubstituted C₆-C₆₀ aryl group, a substituted or unsubstituted C₆-C₆₀ alkyl aryl group, a substituted or unsubstituted C₆-C₆₀ aryl alkyl group, a substituted or unsubstituted C₂-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₆-C₆₀ aryloxy group, a substituted or unsubstituted C₆-C₆₀ arylthio group, or a substituted or unsubstituted C₈-C₆₀ condensed polycyclic group;
L₁ and L₂ are each independently a direct bond, O, S, NR₈;
R₈ is a substituted or unsubstituted C₆-C₆₀ aryl group, a substituted or unsubstituted C₆-C₆₀ alkyl aryl group, a substituted or unsubstituted C₆-C₆₀ aryl alkyl group, a substituted or unsubstituted C₂-C₆₀ heteroaryl group, a substituted or unsubstituted C₂-C₆₀ alkyl heteroaryl group, a substituted or unsubstituted C₂-C₆₀ heteroaryl alkyl group, or R₈ is combined with Ar₁, Ar₂ or R₆ to form a substituted or unsubstituted C₂-C₆₀ nitrogen-containing heteroaryl ring;
m, a₁ and a₂ are each independently an integer of 0 to 4 and n is an integer of 0 to 3;
p is an integer of 1 to 3; and
when n is 2 or more, each R₂ is different or the same,
when m is 2 or more, each R₃ is different or the same,
when p is 2 or more, each Ar₂ is different or the same,
when a₁ is 2 or more, each L₁ are different or the same, or
when a₂ is 2 or more, each L₂ is different or the same.

11. The light-emitting device of claim 10, wherein the intermediate layer comprises an emission layer and at least one organic layer, and at least one of the emission layer or the organic layer includes the heterocyclic compound.

12. The light-emitting device of claim 11, wherein the emission layer emits a blue light having a maximum emission central wavelength in a range from 420 nm to 490 nm.

13. The light-emitting device of claim 10, wherein the intermediate layer comprises:
a hole transfer region comprising at least one of a hole injection layer, a hole transport layer and an electron blocking layer;
an emission layer; and
an electron transfer region comprising at least one of a hole blocking layer, an electron transport layer, and an electron injection layer,
wherein at least one of the hole transfer region, the emission layer, or the electron transfer region includes the heterocyclic compound.

14. The light-emitting device of claim 13, wherein the emission layer includes a host and a dopant, and the heterocyclic compound is included as a host for a phosphorescent device, or a thermally activated delayed fluorescence host.

15. The light-emitting device of claim 14, wherein the dopant further includes a phosphorescent dopant.

16. An electronic device comprising the light-emitting device according to claim 10.

17. The electronic device of claim 16, wherein the electronic device comprises a display device comprising the light-emitting device, a processor, a memory, and a power module.

18. The electronic device of claim 17, wherein the processor comprises at least one selected from the group consisting of a central processing unit, an application processor, a graphic processing unit (GPU), a communication processor, an image signal processor, and a controller.

19. The electronic device of claim 17, wherein the power module comprises a power adapter or a battery.

20. The electronic device according to claim 16, wherein the electronic device is a flat panel display, a curved display, a computer monitor, a medical monitor, a television, a billboard, a light for indoor or outdoor lighting, a signal lighting, a head-up display, a full or partial transparent display, a flexible display, a rollable display, a foldable display, a stretchable display, a laser printer, a phone, a mobile phone, a tablet, a phablet, a personal information terminal, a wearable device, a laptop computer, a digital camera, a camcorder, a viewfinder, a micro display, a 3D display, a virtual reality or augmented reality display, a vehicle, a video wall including multiple displays tiled together, a theater or stadium screen, a phototherapy device, or a signage.
